(19) United States
(12) Patent Application Publication
(10) Pub. No.: US 2013/0252843 A1
(43) Pub. Date: Sep. 26, 2013

(54) METHOD OF MAKING AND USING FLUORESCENT-TAGGED NANOPARTICLES AND MICROARRAYS

(71) Applicants: Mingdi Yan, Tewksbury, MA (US); Xin Wang, Portland, OR (US); H. S. N. Jayawardena, Lowell, MA (US)

(72) Inventors: Mingdi Yan, Tewksbury, MA (US); Xin Wang, Portland, OR (US); H. S. N. Jayawardena, Lowell, MA (US)

(73) Assignee: THE STATE OF OREGON ACTING BY AND THROUGH THE STATE BOARD OF HIGHER EDUCATION ON BEHALF OF PORTL, Portland, OR (US)

(21) Appl. No.: 13/988,495

(22) PCT Filed: Nov. 22, 2011
(86) PCT No.: PCT/US2011/061936

§ 371 Date: May 20, 2013

Related U.S. Application Data
(60) Provisional application No. 61/416,186, filed on Nov. 22, 2010.
(60) Provisional application No. 61/463,878, filed on Feb. 23, 2011.
(60) Provisional application No. 61/571,503, filed on Jun. 28, 2011.

(51) Int. Cl. G01N 33/58

(52) U.S. Cl. CPC G01N 33/587
USPC 506/9; 436/94; 436/86; 506/32

(57) ABSTRACT

Disclosed embodiments concern differentiating and classifying one or more targets using a perhalophenylazide-derived nanoparticle probe, or multiple such probes. Particular embodiments concern using statistical analysis to produce score plots illustrating the level of differentiation and/or classification. Also disclosed are methods for making perhalophenylazide-derived nanoparticle probes, individually or by using a microarray technique. Particular embodiments concern methods for using the per halophenylazide-derived nanoparticle probes to diagnose, detect, and/or treat a disease. Kits comprising the perhalophenylazide-derived nanoparticle probes are also disclosed.

### CROSS REFERENCE TO RELATED APPLICATION

This application claims the benefit of U.S. Provisional Application No. 61/416,186, filed on Nov. 22, 2010, U.S. Provisional Application No. 61/463,878, filed on Feb. 23, 2011, and U.S. Provisional Application No. 61/571,503, filed Jun. 28, 2011. The entire disclosure of the provisional applications is considered to be part of the disclosure of the following application and are hereby incorporated by reference.

### ACKNOWLEDGMENT OF GOVERNMENT SUPPORT

This invention was made with government support under Grant Nos. R01GM080295 and 2R15GM066279 awarded by the National Institutes of Health. The United States government has certain rights in the invention.

### FIELD

The present disclosure concerns conjugates comprising perhalophenylazide-derived nanoparticles labeled with molecular probes, embodiments of a method for making and a method for using such conjugates, and microarrays and kits comprising such conjugates.

### BACKGROUND

A major challenge in bioanalysis, including high-throughput screening, is signal transduction, i.e., effectively displaying the outcome of ligand-receptor interactions. Fluorescence is by far the most commonly used detection method. Fluorescence labeling involves conjugating a fluorescent tag to a ligand to be studied. Examples of fluorescent tags include organic dyes, which continue to be widely used in view of their diverse structures, functionalities, solubility, and spectral properties.

A major drawback of organic dyes, however, is their relatively poor photostability. When exposed to light, organic dyes can photobleach, resulting in decreased fluorescence intensity. Another drawback is that certain biological molecules, such as sugars and glycans, are complex molecules that are difficult to label with currently known techniques. Glycans play key roles in cell-based recognition, and have been implicated in many proliferative cell disease processes, including cancer. These molecules are therefore are highly suited for probing cell surface properties and early detection of cancer.

Cancer cells are physically different not only from their normal counterparts, but also among themselves, depending on various factors, such as the stages of development and malignancy. Therefore, comprehensive knowledge of all these differences can improve cancer detection and prognosis. Cancer cell surface lipids, proteins, and their modifications play important roles in mediating cancer development and therapeutic responses. However, knowledge of cell surface structures is very limited, which hinders the development of novel approaches in cancer detection, diagnosis, prognosis, and individualized therapy decision-making.

Most cancer detection prognoses and decisions concerning treatment plans are biomarker driven, based on specific genetic and pathological features. Although cancer cells are known to be biophysically different from normal cells, determining how to use these differences to improve cancer detection and treatment is relatively unknown.

Detecting cancer more accurately or at earlier stages could lead to better prognoses for many cancer patients. Pancreatic cancer is one example of disease that, if diagnosed at an earlier stage, could have a better prognosis. Carbohydrate markers for pancreatic cancer are known, such as the CA 19-9 antigen, but the current analytical methods are not optimal for various reasons, including low throughput and reproducibility. As a result, a high throughput, affinity-based method that is practical for multiplexed studies is required.

### SUMMARY

Disclosed embodiments concern a method of differentiating and classifying at least one target, comprising exposing a sample to at least one perhalophenylazide-derived nanoparticle probe; and detecting a signal produced by a specific or nonspecific interaction between the perhalophenylazide-derived nanoparticle probe and the target. Particular embodiments concern targets selected from cell lines, proteins, nucleic acids, and combinations thereof. Cell lines, such as prostate cell lines, breast cancer cell lines, pancreatic cell lines, and combinations thereof, can be selected from cells lines in a normal state, cancer state, malignant state, chemosensitive state, and combinations thereof. Additional targets include proteins, such as lectins. Exemplary disclosed lectins include mannose binding lectins, galactose/N-acetylgalactosamine binding lectins, N-acetylglucosamine binding lectins, N-acetylneuraminic acid binding lectins, fucose binding lectins, and combinations thereof.

Particular embodiments concern perhalophenylazide-derived nanoparticle probes (or conjugates), comprising a perhalophenylazide moiety, a nanoparticle, and a molecular probe. In particular embodiments, the perhalophenylazide moiety has a formula

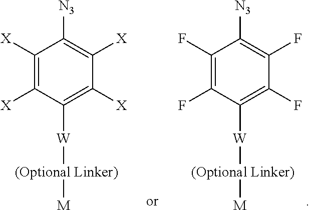

In particular embodiments, the perfluorophenylazide moieties can have the following formulas

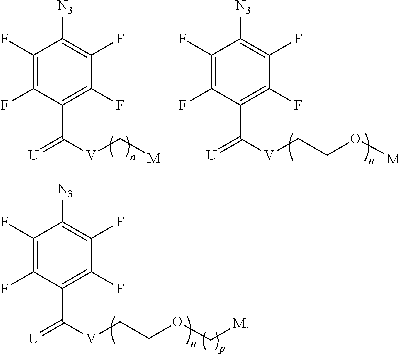

In certain embodiments the perhalophenylazide moiety can have a formula

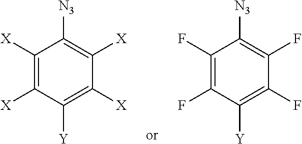

where X may be a halogen, and Y may be selected from a heteroatom-containing moiety capable of undergoing further chemical manipulation to make a perhalophenylazide moiety as disclosed herein. A person of ordinary skill in the art will recognize that certain recitations of Y include functional groups that may be converted, through additional chemical transformations, to a different functional group suitable for subsequent coupling reactions. For example, if Y is a halogen, a carbonylation reaction, such as a stannane-mediate carbonylation or palladium-mediated carbonylation, with a suitable coupling partner may be used to convert the halogen to a carbonyl-containing moiety, such as an aldehyde or an ester.

Disclosed perhalophenylazide moieties can be further modified using a moiety having a general formula illustrated below

Z-(Optional Linker)-M

where M and the optional linker are both as previously recited; Z may be a nucleophilic group or an electrophilic group. A person of ordinary skill in the art will recognize that in embodiments where Z is electrophilic, Y may be selected from nucleophilic heteroatom-containing moieties; and when Z is nucleophilic, Y may be selected from electrophilic heteroatom-containing moieties.

In particular embodiments, the nanoparticle may be selected from a metalloid nanoparticle, a metal oxide nanoparticle, a metal nanoparticle, a lanthanum-containing nanoparticle, an organic nanoparticle, and combinations thereof. Certain embodiments concern using a silica nanoparticle, a titania nanoparticle, a zinc oxide nanoparticle, a yttrium vanadium oxide nanoparticle, a gold nanoparticle, a silver nanoparticle, a lanthanum phosphate nanoparticle, a polystyrene nanoparticle, a graphene nanoparticle, and combinations thereof. In particular embodiments, the nanoparticle has a diameter ranging from about 1 to about 200 nm; more typically from about 5 to about 100 nm. Exemplary nanoparticles include gold nanoparticles and silica nanoparticles. In particular embodiments, the nanoparticle further comprises a signal generating moiety, such as a fluorescent dye.

In certain embodiments of the disclosed nanoparticle probes, the molecular probe may be selected to interact with a target and is selected from a biomolecule, a pharmaceutical compound, and any combination thereof. In particular embodiments, the biomolecule may be selected from an antibody, a carbohydrate, an amino acid, an amino acid oligomer, a protein, a nucleic acid, a nucleic acid oligomer, RNA, DNA, a lipid, and combinations thereof. When the biomolecule is a carbohydrate, it may be selected from a monosaccharide, a disaccharide, an oligosaccharide, and a polysaccharide; particular embodiments include Galβ1-4Glc, Glcβ1-4Glc, Glcα1-2Glc, GlcNAcβ1-4GlcNAc, Manα1-2Man, Manα1-2Manα1-2Man, Galα1-3(Fucα1-2)Galβ1-3GlcNAcβ, Neu5Acα2-3Galβ1-4Glcβ, Galβ1-3GalNAc, Fucα1-2Gal-R, GalNAcαSer/Thr, Galα1-3Gal-R, Galβ1-3(Fucα1-4)GlcNAc-R, Galβ1-4(Fucα1-3)GlcNAc-R, GalNAcα1-3GalNAcβ1-R, and 3Galα1-4Galβ1-4Glc-R.

In particular embodiments, the perhalophenylazide-derived nanoparticle probe has a formula

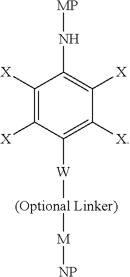

Particular embodiments concern perfluorophenylazide-derived nanoparticle probes having the following formulas

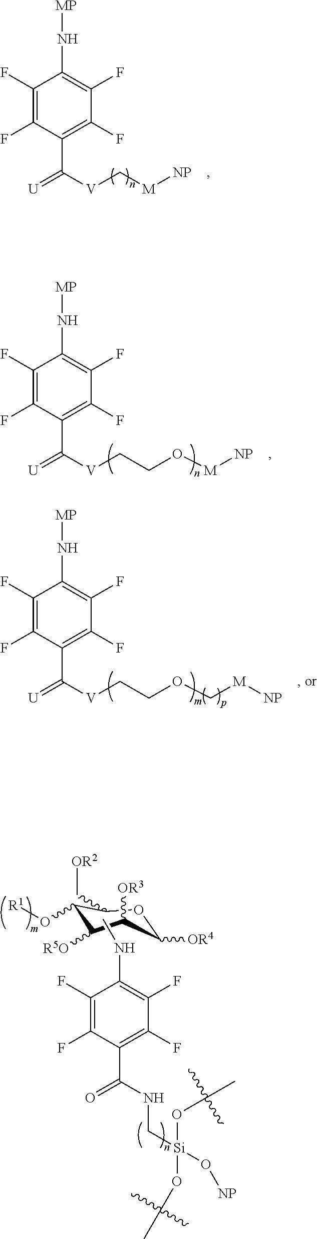

Also disclosed are methods for using the disclosed perhalophenylazide-derived nanoparticle probes. In particular embodiments, a target can be detected and/or classified using the disclosed methods. In certain particular embodiments, multiple targets can be detected, differentiated, classified, as well as combinations thereof, using the disclosed method. Certain embodiments concern exposing a sample to disclosed embodiments of the perhalophenylazide-derived nanoparticle probe and detecting a signal produced by the interaction between the probe and the target. In further embodiments, the signal can be converted to a score plot using statistical analysis, such as linear discriminant analysis.

Embodiments of a method for imaging biological molecules or therapeutic agents also are described. For example, one disclosed embodiment comprises providing a conjugate comprising a nanoparticle, a signal generating moiety, a perhalophenyl azide, and a biological molecule that is one member of a specific binding pair or therapeutic agent. The conjugate is exposed to a sample potentially comprising a second member of the specific binding pair or a therapeutic target. The specific binding pair or therapeutic agent interacting with the target is then imaged, such as by fluorescent microscopy. The method also can include labeling the second member of the specific binding pair or the therapeutic target with a conjugate comprising a nanoparticle, a signal generating moiety, and a perhalophenyl azide. Additional embodiments include exposing a target to plural different conjugates, each conjugate comprising a different signal generating moiety, such as to detect plural different targets on a sample. These embodiments can be realized using a microarray, such as a lectin or carbohydrate microarray, comprising one member of a specific binding pair.

In particular embodiments, detecting comprises using a detection method capable of measuring the interaction between the perhalophenylazide-derived nanoparticle probe and the target. Certain embodiments concern detecting attached targets, detached targets, and combinations thereof. The interaction can be specific, nonspecific, or combinations thereof. Particular embodiments concern using a colormetric method of detection, a fluorescent method of detection, and combinations thereof.

Particular embodiments of the disclosed method concern converting the detected signal to a score plot using linear discriminant analysis. The score plot comprises response patterns for one or more targets and can be used to determine response patterns associated with different physical, biochemical, and/or chemical states of the target. In particular embodiments, the score plot is used to determine response patterns associated with a normal state cell, a cancer state cell, a malignant cell, a chemosensitive cell; and combinations thereof.

The disclosed method also concerns exposing plural different targets to plural different perhalophenylazide-derived nanoparticle probes. In particular embodiments, the target can be exposed to a microarray of perhalophenylazide-derived nanoparticle probes. In particular embodiments, the microarray can comprise a solid support and at least two different glycan binding proteins or at least two different glycans immobilized on the solid support. In particular embodiments, the glycan binding protein comprises a perhalophenylazide-derived nanoparticle probe.

Particular embodiments concern a method of diagnosing and/or treating a disease, comprising exposing a target in a sample to a perhalophenylazide-derived nanoparticle probe; and detecting the signal produced by an interaction between the perhalophenylazide-derived nanoparticle probe and the target. The target can be selected from plural multiple targets. Particular embodiments concern using the disclosed method to diagnose and/or treat diseases such as cancer (e.g. prostate cancer, breast cancer, and pancreatic cancer). In certain embodiments, the sample exists in vivo or in vitro.

Additional disclosed embodiments concern a microarray comprising a solid support, and at least two different glycan binding proteins or two different glycans immobilized on the solid support. The glycan binding protein or glycan can comprise a PHPA-signal generating moiety conjugate. Suitable microarrays can be made by a method comprising providing a solid support, and immobilizing at least two different glycan binding proteins or two different carbohydrates on the solid support using a PHPA. The substrate surface can be modified to prevent or substantially preclude interactions between biological molecules and the substrate. Polymer-based PHPA surfaces can be used to enhance specific interaction signals. Moreover, polymer-PHPA surfaces can be used along with an antifouling coating, and ligands and an anti-fouling coating may be applied at designated locations in a spatially-controlled fashion.

Kits comprising a microarray also are disclosed. The kits can be used for various purposes. One embodiment concerns measuring glycan levels of a protein comprising detecting glycan levels of a protein in a sample using the microarray.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic drawing illustrating one embodiment of a disclosed method for high-throughput synthesis of glycol-labeled fluorescein-doped silica nanoparticle.

FIG. 2 is a schematic drawing that illustrates a nanoparticle probe array for differentiating cell lines by pattern recognition.

FIG. 3 is schematic drawings illustrating synthesis of exemplary PHPA-functionalized nanoparticles.

FIG. 4 is a TEM image of FITC-doped SNPs (scale bar 0.2 μm).

FIG. 5 is an image illustrating size distribution of FITC-doped SNPs as measured by DLS.

FIG. 6 illustrates fluorescence spectra of FITC-doped SNPs before and after UV irradiation for 10 minutes.

FIG. 7 illustrates fluorescence spectra of FITC before and after UV irradiation for 10 minutes.

FIGS. 8(a-d) are fluorescence spectra of FSNP-Man (8a) and fluorescence spectra of FSNP-Gal (8b), before (black lines) and after (red lines) incubating with Con A; dynamic light scattering (DLS) results of Man-FSNPs, before (red) and after (green) binding with Con A (8c); and a transmission electron microscopy (TEM) image of Man-FSNPs after treating with Con A (8d).

FIGS. 9(a-b) are TEM images after FSNP-Man was treated with E. coli strain ORN178 (9a), or ORN208 (9b), where the insert is a fluorescence image of the corresponding sample (scale bars=500 nm).

FIGS. 10(a-c) are images illustrating the following: a schematic illustration of one embodiment of a method for preparing a lectin microarray, incubation with FSNP-Man, and fluorescence imaging (10a); a fluorescence image (10b); and a graph of fluorescence intensities from the lectin microarray after treating with FSNP-Man, where each data point was the average of the 7 spots on the microarray (10c).

FIG. 11 is a schematic drawing illustrating one embodiment of a method for making a making a carbohydrate array and subsequent interaction with FSNP-labeled Con A.

FIGS. 12(a-c) are fluorescence images of carbohydrate array where the polymer coating was PEOX (12a), and PS (12b), respectively, and the array, without any polymer coating, was treated with BSA before FSNP-Con A (12c).

FIG. 13 is a schematic diagram illustrating fabrication of a polymer array on PFPA-functionalized surface.

FIG. 14(a-c) illustrates fluorescent images of immobilized polymers after treating with FSNPs: a) PEOX 500 k, b) PEO 300 k, c) PS 280 k.

FIG. 15 illustrates one embodiment of a method for preparing PLL-PFPA and PFPA surfaces and subsequent immobilization of carbohydrates.

FIG. 16 is a vapor condensation image of an array containing 21 different carbohydrates fabricated on PLL-PFPA surface, where each carbohydrate was spotted in triplicate.

FIGS. 17(a-d) are surface plasma resonance images (SPRi) (17a,b) and fluorescence images (17c,d) of a carbohydrate array after treating with Con A and FITC-labeled Con A, respectively, where the array was prepared on either PFPA surface (17a, c), or PLL-PFPA surface (17b, d), where the carbohydrates are Gal (1), Man (2), maltoheptaose (3), 10 kD dextran (4), 60-90 kD dextran (5), and β-cyclodextrin (6), respectively, and where the line profiles represent the relative signal intensities.

FIG. 18 illustrates one embodiment of a method for making PAAm-PFPA surfaces and subsequent immobilization.

FIG. 19 illustrates one embodiment of a method for making PAAm-PFPA surfaces and subsequent immobilization.

FIG. 20 is an AFM image of silica nanoparticles immobilized on a PFPA surface (scale bar: 500 nm).

FIG. 21 is an AFM image of silica nanoparticles immobilized on a PAAm-PFPA surface (scale bar: 500 nm).

FIG. 22 provides examples of various commercially available thiol and silane structures useful for functionalization.

FIG. 23 illustrates one embodiment of method for making covalently immobilized graphene films on a PHPA surface.

FIGS. 24(a-c) Interactions of Man2-FSNPs with super-microarray: fluorescence intensity vs. the printing concentration of Con A (□) (24a); fluorescence intensity vs. the concentration of Man2-FSNPs (∘) (24b); and fluorescence intensity vs. Man2-FSNPs (1.5 mg/mL) incubation time (24c) (inserts: fluorescence images of Con A spots (top panel) and SBA spots (bottom panel)).

FIGS. 25(a-d) are images illustrating: fluorescence images of a printed Con A spot after incubation with Man2-FSNP for 2 h (25a); and AFM images of the printed Con A spot (25b-d).

FIG. 26 is a schematic drawing illustrating lectin super-microarray fabrication and subsequent assays with the disclosed conjugate.

FIGS. 27(a-d) are a fluorescence image (27a, c) and fluorescence intensity (27b, d) of lectin super-microarrays interacting with glycol-SFNPs.

FIGS. 28(a-d) illustrate: fluorescence image of a lectin super-microarray after incubating with Man2-FSNP and varying concentrations of 2α-Man2 (28a); fluorescence intensities vs. free 2α-Man2 concentration (28b-d) for CVN-Q CVN-M (28c) and Con A (28d).

FIGS. 29(a-b) are images illustrating a TEM image of FITC-doped silica nanoparticles (29a); and DLS analysis of FITC-doped silica nanoparticles (29b).

FIGS. 30(a-f) are fluorescent microscopy images of: non-blocked lactose-conjugated nanoparticles on PC3 (30a); non-blocked cellobiose-conjugated nanoparticles on PC3 (30b); non-blocked lactose-conjugated nanoparticles on BPH1 (30c); BSA-blocked lactose-conjugated nanoparticles on PC3 (30d); BSA-blocked cellobiose-conjugated nanoparticles on PC3 (30e); and BSA-blocked lactose-conjugated nanoparticles on BPH1 (30f).

FIGS. 31(a-c) are fluorescent microscopy images of: lactose-conjugated FSNPs with PC3 (31a); non-ligand-conjugated FSNPs with PC3 (31b); and lactose-conjugated FSNPs with BPH1 (31c).

FIG. 32 is an image illustrating fluorescent intensity measurements of FSNP-bound PC3 cells. The inset shows the fluorescent spectra (emission scan) of original nanoparticle solutions.

FIG. 33 is an image of western blots of galectin-1 expression in pure galectin-1 solutions after being mixed with nanoparticles.

FIG. 34 is an image illustrating the change in the weight of Gal-1 bound to FSNPs as a function of FSNP concentrations, fit (solid curve) into a Langmuir adsorption isotherm (eq. 1).

FIG. 35 is a schematic drawing illustrating one embodiment of a method for making lectin “super-microarrays,” subsequent incubations with sugar-conjugated FITC-SNPs, and simultaneous screening of multiple arrays on a single slide.

FIGS. 36(a-b) illustrate a schematic drawing illustrating high-throughput screening using “super-microarrays” (36a), and competition assays with free ligands for affinity determinations (36b), as discussed in the Examples, such as Example 9.

FIG. 37 are fluorescence images of “super-microarray” interactions in Lectin Group 1 (Man3: α-1,2-α-1,2-mannotriose; Man2: α-1,2-mannobiose)

FIG. 38 are normalized fluorescence intensities for the Lectin Group 1 results.

FIG. 39 are fluorescence images of “super-microarray” interactions in Lectin Group 2 (Man3′: α-1,3-α-1,6-mannotriose; Man2′: α-1,6-mannobiose)

FIG. 40 illustrates normalized fluorescence intensities for the Lectin Group 2 results.

FIGS. 41(a-d) illustrate (41a) fluorescence images of “super-microarray” competition interactions with fixed amount of Man2-conjugated FSNP and varying concentrations of free Man2 competitors; (41b-d) fluorescent intensities versus free Man2 concentration, for interactions with CVN-Q (b), CVN-M (c) and Con A (41d), along with corresponding dissociation constants (K_{D}).

FIG. 42 is a schematic diagram illustrating one embodiment of a method for immobilizing a polymer on a substrate surface, such as a wafer or glass slide, as discussed in Example 13.

FIG. 43 concerns silica nanoparticle adsorption evaluation by fluorescence imaging as discussed in Example 13.

FIGS. 44-45 illustrate fluorescence image results obtained after incubating a polymer array on a glass slide with FITC-doped silica nanoparticles.

FIGS. 46-47 illustrate fluorescence image results obtained after incubating a polymer array on a wafer with FITC-doped silica nanoparticles.

FIGS. 48-49 illustrate fluorescence image results obtained after incubating a polymer array on a wafer with FITC-doped silica nanoparticles.

FIGS. 50-51 illustrate fluorescence image results obtained after incubating a polymer array on a wafer (pre-treated with BSA solution in PBS) with FITC-doped silica nanoparticles.

FIGS. 52(a-b) illustrate fluorescence images of carbohydrate microarrays with PS coating (52a) and PEO coating (52b).

FIG. 53 provides fluorescence intensities of carbohydrate microarrays after treating with ConA-FSNPs.

FIG. 54 is a bar graph illustrating the absorbance changes (y-axis) of non-specific interactions between particular embodiments of perhalophenylazide-derived nanoparticle probes (x-axis) and three different lectins.

FIG. 55 is a factor score plot that illustrates the differentiation of three different lectins that have non-specific interactions with particular embodiments of perhalophenylazide-derived nanoparticle probes.

FIG. 56 is a bar graph illustrating the absorbance changes (y-axis) of specific interactions between particular embodiments of perhalophenylazide-derived nanoparticle probes (x-axis) and four different lectins.

FIG. 57 is a factor score plot that illustrates the differentiation of four different lectins that have specific interactions with particular embodiments of perhalophenylazide-derived nanoparticle probes.

### DETAILED DESCRIPTION

### I. Terms and Definitions

Unless otherwise noted, technical terms are used according to conventional usage. Definitions of common terms in molecular biology may be found in Benjamin Lewin, Genes VII, published by Oxford University Press, 2000 (ISBN 019879276X); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

As used herein, the singular terms “a,” “an,” and ‘The” include plural referents unless context clearly indicates otherwise. Similarly, the word “or” is intended to include “and” unless the context clearly indicates otherwise. Also, as used herein, the term “comprises” means “includes.” Hence “comprising A or B” means including A, B, or A and B.

A wavy line ( [symbol] ) indicates a bond disconnection. A dashed line (- - - - -) illustrates that a bond may be formed at a particular position.

Nucleotide sizes or amino acid sizes, and all molecular weight or molecular mass values, stated herein are approximate, and are provided for description.

The materials, methods, and examples provided are illustrative only and not intended to be limiting. Although methods and materials similar or equivalent to those described herein can be used in the practice or testing of the present disclosure, suitable methods and materials are described below.

All publications, patent applications, patents, and other references mentioned herein are incorporated by reference in their entirety. In case of conflict, the present specification, including explanations of terms, will control.

In order to facilitate review of the various examples of this disclosure, the following explanations of specific terms are provided:

Addition reaction: When referring to nitrene reactions, this term generally refers to various addition and insertion reactions that nitrenes can undergo with other molecules, such as biological molecules.

Aliphatic: Any open or closed chain molecule, excluding aromatic compounds, containing only carbon and hydrogen atoms which are joined by single bonds (alkanes), double bonds (alkenes), or triple bonds (alkynes). This term encompasses branched aliphatic compounds, linear aliphatic compounds, saturated aliphatic compounds, unsaturated aliphatic compounds, and combinations thereof.

Antibody: “Antibody” collectively refers to immunoglobulins or immunoglobulin-like molecules (including by way of example and without limitation, IgA, IgD, IgE, IgG and IgM, combinations thereof, and similar molecules produced during an immune response in any vertebrate, for example, in mammals such as humans, goats, rabbits and mice) and antibody fragments that specifically bind to a molecule of interest (or a group of highly similar molecules of interest) to the substantial exclusion of binding to other molecules (for example, antibodies and antibody fragments that have a binding constant for the molecule of interest that is at least 10³ M⁻¹ greater, at least 10⁴ M⁻¹ greater or at least 10⁵ M⁻¹ greater than a binding constant for other molecules in a biological sample.

More particularly, “antibody” refers to a polypeptide ligand comprising at least a light chain or heavy chain immunoglobulin variable region which specifically recognizes and binds an epitope of an antigen. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody.

Aryl: A substantially hydrocarbon-based aromatic compound, or a radical thereof (e.g. C₆H₅) as a substituent bonded to another group, particularly other organic groups, having a ring structure as exemplified by benzene, naphthalene, phenanthrene, anthracene, etc. This term also encompasses aryl compounds comprising aliphatic substituents.

Aryl alkyl: A compound, or a radical thereof (C₇H₇ for toluene) as a substituent bonded to another group, particularly other organic groups, containing both aliphatic and aromatic structures.

Binding affinity: The tendency of one molecule to bind (typically non-covalently) with another molecule, such as the tendency of a member of a specific binding pair for another member of a specific binding pair. A binding affinity can be measured as a binding constant, which binding affinity for a specific binding pair (such as an antibody/antigen pair or nucleic acid probe/nucleic acid sequence pair) can be at least 1×10⁵ M¹, such as at least 1×10⁶ M⁻¹, at least 1×10⁷ M¹ or at least 1×108 M⁻¹. In one embodiment, binding affinity is calculated by a modification of the Scatchard method described by Frankel et al., Mol. Immunol., 16:101-106, 1979. In another embodiment, binding affinity is measured by an antigen/antibody dissociation rate. In yet another embodiment, a high binding affinity is measured by a competition radioimmunoassay. In several examples, a high binding affinity for an antibody/antigen pair is at least about 1×10⁸M⁻¹. In other embodiments, a high binding affinity is at least about 1.5×10⁸ M⁻¹, at least about 2.0×10⁸ M⁻¹, at least about 2.5×10⁸ M⁻¹, at least about 3.0×10⁸ M⁻¹, at least about 3.5×10⁸ M⁻¹, at least about 4.0×10⁸ M⁻¹, at least about 4.5×10⁸ M⁻¹, or at least about 5.0×10⁸ M⁻¹.

Carbohydrate: An organic compound having an empirical formula Cₘ(H₂O)ₙ. Carbohydrates, synonymous with saccharides, are divided into four chemical groupings: monosaccharides, disaccharides, oligosaccharides, and polysaccharides.

Classif(y/ying/ication/ied): Arranging one or more targets into classes according to shared qualities. For example, one or more cell lines can be classified into groups or classes based on the state of the cell or cells; examples of cell classes include, but are not limited to, normal, cancer, malignant, and/or chemosensitive.

Conjugate: A molecule comprising two or more independent molecules, which have been joined through a bond (typically a covalent or ionic bond). The conjugate may also be a probe, such as a nanoparticle probe, disclosed herein.

Conjugating, joining, bonding or linking: Joining one molecule to another molecule to make a larger molecule.

Coupl(ed/ing): The term “coupled” means joined together, either directly or indirectly. A first atom or molecule can be directly coupled or indirectly coupled to a second atom or molecule. Coupling encompasses covalent coupling, electrostatic coupling, polar-covalent coupling, and ionic coupling.

Detectable Label: A detectable compound or composition that is attached directly or indirectly to another molecule to facilitate detection of that molecule, such as, without limitation, an amino acid, protein, antibody, nucleotide, nucleic acid, sugar or carbohydrate. Specific, non-limiting examples of labels include fluorescent tags, enzymes, and radioactive isotopes.

Derivative: In chemistry, a derivative is a compound that is derived from a similar compound or a compound that can be imagined to arise from another compound, for example, if one atom is replaced with another atom or group of atoms. The latter definition is common in organic chemistry. In biochemistry, the word is used for compounds that at least theoretically can be formed from the precursor compound.

Different(iate/ation/ating): This term encompasses recognizing and/or identifying what makes two or more targets different. For example, disclosed methods can be used to differentiate between multiple cell lines or between characteristics of one or more cells in one or more cell lines.

Epitope: An antigenic determinant. These are particular chemical groups or contiguous or non-contiguous peptide sequences on a molecule that are antigenic, that is, that elicit a specific immune response. An antibody binds a particular antigenic epitope.

Fluorophore, Fluorescent, Fluorescent dye: A fluorophore is a component of a molecule which causes a molecule to be fluorescent. Fluorescence refers to the emission of radiation, especially of visible light, by a substance as a result of exposure to external radiation, particularly light, and even more particularly light of a defined wavelength or wavelength range. For example, a molecule may include a functional group that absorbs energy of a specific wavelength and re-emits energy at a different (but equally specific) wavelength. The amount and wavelength of the emitted energy depend on both the fluorophore and the chemical environment. Fluorophores are particularly important in biochemistry, protein, nucleic acid, and carbohydrate studies.

Glycan: An oligosaccharide or polysaccharide, such as an oligosaccharide or polysaccharide comprising O-glycosidic linkages of monosaccharides. Cellulose is a glycan (specifically a glucan) comprising β-1,4-linked D-glucose. As another example, chitin is a glycan comprising β-1,4-linked N-acetyl-D-glucosamine. Glycans can be homo- or heteropolymers of monosaccharide residues. Glycans can be linear or branched molecules.

Glucan: A polysaccharide comprising D-glucose monomers coupled by glycosidic bonds.

Heteroatom: An atom other than carbon and hydrogen, such as, but not limited to, oxygen, sulfur, nitrogen, phosphorus, chlorine, fluorine, bromine, iodine, and selenium.

Heteroatom-containing moiety: A “heteroatom-containing moiety” comprises at least one heteroatom.

Heterobifunctional: Cross-linking agents containing at least two different reactive groups at each end, which are reactive towards numerous groups, including but not limited to sulfhydryls and amines, and create chemical covalent bonds between two or more molecules.

Isolated: An “isolated” microorganism (such as a virus, bacterium, fungus, or protozoan) has been substantially separated or purified away from microorganisms of different types, strains, or species. Microorganisms can be isolated by a variety of techniques, including serial dilution and culturing.

An “isolated” biological component (such as a nucleic acid molecule, protein or organelle) has been substantially separated or purified away from other biological components in the cell of the organism in which the component naturally occurs, such as other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant expression in a host cell, as well as chemically synthesized nucleic acids or proteins, or fragments thereof.

Lectin: A sugar-binding protein highly specific for sugar moieties. Lectins also are involved in biological recognition between cells and proteins. For example, some viruses use lectins for attachment to host organism cells during infection.

Lectins have various uses, including medicinal and medical research, such as blood typing; tracing efferent axon paths with PHA-L; a lectin from the kidney bean; BanLec from bananas inhibits HIV in vitro; studying carbohydrate recognition by proteins; understanding how proteins recognize carbohydrates; Concanavalin A and other commercially available lectins have been used in affinity chromatography for purifying glyco proteins, and in general, proteins may be characterized with respect to glycoforms and carbohydrate structure by means of affinity chromatography, blotting, affinity electrophoresis and affinity immunoelectrophoresis with lectins. Exemplary lectins include concanavalin A (conA), lentil lectin (LCH), snowdrop lectin (GNA), ricin (RCA), peanut agglutinin (PNA), Jacalin (AIL), hairy vetch lectin (VVL), wheat germ agglutinin (WGA), elderberry lectin (SNA), Maackia amurensis leukoagglutinin (MAL), maackia amurensis hemoagglutinin (MAH).

Linker: As used herein, a linker is a molecule or group of atoms positioned between two moieties. The linkers may be aliphatic, having a formula —(CH₂)_{q}—, where q ranges from about 1 to about 20, or they comprise alkylene glycol units. In other embodiments, linkers are bifunctional, i.e., the linker includes a functional group at each end, wherein the functional groups are used to couple the linker to the two moieties. The two functional groups may be the same, i.e., a homobifunctional linker, or different, i.e., a heterobifunctional linker.

Lower alkyl: Any aliphatic chain that contains 1-10 carbon atoms.

Molecule of interest or target: A molecule for which the presence, location and/or concentration is to be determined, particularly biological molecules. Examples of molecules of interest include proteins, nucleic acid sequences, sugars, carbohydrates, glycans, etc.

Monoclonal antibody: An antibody produced by a single clone of Lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

Nanoparticle: A nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohorns, nano-onions, nanorods, nanoropes and quantum dots. Examples of nanoparticles include metal oxide nanoparticles, such as silica nanoparticles, titania nanoparticles, and zinc oxide nanoparticles, metal nanoparticles, such as gold nanoparticles and silver nanoparticles, and combinations of such particles.

Nitrenogenic group: A chemical moiety that, when exposed to a reaction-energy source, becomes a nitrene group.

Nitrene group (or “nitrene” or “nitrene intermediate”): The nitrogen analogs of carbenes. Like carbenes, nitrenes are generally regarded as intermediates. Important nitrene reactions include: (a) addition reactions at CH sites and NH sites, and addition at C—C double and triple bonds.

Polypeptide: A polymer in which the monomers are amino acid residues that are joined together through amide bonds. When the amino acids are α-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Proliferative disease: Abnormal and uncontrolled cell growth. Neoplasia is one example of a proliferative disorder. A neoplasm is an abnormal growth of tissue that results from excessive cell division. Examples of hematological tumors include leukemias, such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

Protein: A naturally occurring or synthesized molecule comprised of amino acids (e.g. a polypeptide).

Purified: The term “purified” does not require absolute purity; rather, it is intended as a relative term. Thus, for example, a purified peptide, protein, conjugate, or other active compound is one that is isolated in whole or in part from proteins or other contaminants. Generally, substantially purified peptides, proteins, conjugates, or other active compounds for use within the disclosure comprise more than 80% of all macromolecular species present in a preparation prior to admixture or formulation of the peptide, protein, conjugate or other active compound with a pharmaceutical carrier, excipient, buffer, absorption enhancing agent, stabilizer, preservative, adjuvant or other co-ingredient in a complete pharmaceutical formulation for therapeutic administration. More typically, the peptide, protein, conjugate or other active compound is purified to represent greater than 90%, often greater than 95% of all macromolecular species present in a purified preparation prior to admixture with other formulation ingredients. In other cases, the purified preparation may be essentially homogeneous, wherein other macromolecular species are not detectable by conventional techniques.

Quantum dot: A nanoscale particle that exhibits size-dependent electronic and optical properties due to quantum confinement. Quantum dots have, for example, been constructed of semiconductor materials (e.g., cadmium selenide and lead sulfide) and from crystallites (grown via molecular beam epitaxy), etc. A variety of quantum dots having various surface chemistries and fluorescence characteristics are commercially available from Invitrogen Corporation, Eugene, Oreg. (see, for example, U.S. Pat. Nos. 6,815,064, 6,682,596 and 6,649,138, each of which patents is incorporated by reference herein). Quantum dots are also commercially available from Evident Technologies (Troy, N.Y.). Other quantum dots include alloy quantum dots such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAlAs, and InGaN quantum dots (Alloy quantum dots and methods for making the same are disclosed, for example, in US Application Publication No. 2005/0012182 and PCT Publication WO 2005/001889).

Sample: A biological specimen containing biological molecules. Examples include, but are not limited to, peripheral blood, urine, saliva, tissue biopsy, surgical specimen, amniocentesis samples and autopsy material.

Signal generating moiety: Any atom, or group of atoms, that emits, or can be caused to emit, a detectable signal or that can be detected, such as by using a specific binding pair. For example, the signal generating moiety can be a chromogen, that is a compound that is, or can be converted into, a colored compound. The signal generating moiety also can be fluorescent compound.

Specifically binds: An agent that preferentially binds to a defined target (such as an glycan to lectin). A specific binding agent binds substantially only to a defined target. A minor degree of non-specific interaction may occur between a specific binding agent and a target.

Specific binding moiety: A member of a specific-binding pair. Specific binding pairs bind to each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins (for example, antibodies, lectins, avidins such as streptavidins, and protein A).

Therapeutic agent: A therapeutic agent is any agent that has a therapeutic benefit when administered to a subject, and includes but is not limited to, nucleic acids or amino acid sequences, nucleic acids or amino acid derivatives, peptidomimetic drugs, antibiotics, vitamins, bronchodilators, anti-gout agents, anti-hypertensive agents, diuretic agents, anti-hyperlipidemic agents or ACE inhibitors, anti-tumor agents, histamine (H2) blockers, bismuth salts, synthetic prostaglandins, prazosin, ketanserin, guanabenz acetate, captopril, captopril hydrochloride, enalapril, enalapril maleate, lysinopril, hydralazide, methyldopa, methyldopa hydrochloride, levodopa, carbidopa, benserazide, amlodipine, nitrendipine, nifedipine, nicardipine, verapamil, acyclovir, inosine, pranobex, tribavirine, vidarabine, zidovudine, AZT, aluminum hydroxide, magnesium carbonate, magnesium oxide, sucralphate, sodium carbenoxolone, pirenzepin, loperamide, cimetidine, ranitidine, famotidine, misoprostol, omeprazol, AIDS adjunct agents, alcohol abuse preparations, Alzheimer's disease management agents, amyotrophic lateral sclerosis active ingredient agents, analgesics, anesthetics, antacids, antiarythmics, anticonvulsants, antidepressants, antidiabetic agents, antiemetics, antidotes, antifibrosis active ingredient agents, antifungals, antihistamines, antihypertensives, anti-infective agents, antimicrobials, antineoplastics, antipsychotics, antiparkinsonian agents, antirheumatic agents, appetite stimulants, appetite suppressants, blood modifiers, bone metabolism regulators, cardioprotective agents, cardiovascular agents, central nervous system stimulants, cholinesterase inhibitors, contraceptives, cystic fibrosis management agents, deodorants, diagnostics, dietary supplements, diuretics, dopamine receptor agonists, endometriosis management agents, enzymes, erectile dysfunction active ingredients, fatty acids, gastrointestinal agents, Gaucher's disease management agents, gout preparations, homeopathic remedies, hormones, hypercalcemia management agents, hypnotics, hypocalcemia management agents, immunomodulators, immunosuppressives, ion exchange resins, levocarnitine deficiency management agents, mast cell stabilizers, migraine preparations, motion sickness products, multiple sclerosis management agents, muscle relaxants, narcotic detoxification agents, narcotics, nucleoside analogs, non-steroidal anti-inflammatory drugs, obesity management agents, osteoporosis preparations, oxytocics, parasympatholytics, parasympathomimetics, phosphate binders, porphyria agents, psychoactive ingredient agents, radio-opaque agents, psychotropics, sclerosing agents, sedatives, sickle cell anemia management agents, smoking cessation aids, steroids, stimulants, sympatholytics, sympathomimetics, Tourette's syndrome agents, tremor preparations, urinary tract agents, vaginal preparations, vasodilators, vertigo agents, weight loss agents, Wilson's disease management agents, with specific examples of such agents including abacavir sulfate, abacavir sulfate/lamivudine/zidovudine, acetazolamide, acyclovir, albendazole, albuterol, aldactone, allopurinol, amoxicillin, amoxicillin/clavulanate potassium, amprenavir, atovaquone, atovaquone and proguanil hydrochloride, atracurium besylate, beclomethasone dipropionate, berlactone betamethasone valerate, bupropion hydrochloride, bupropion hydrochloride, carvedilol, caspofungin acetate, cefazolin, ceftazidime, cefuroxime, chlorambucil, chlorpromazine, cimetidine, cimetidine hydrochloride, cisatracurium besilate, clobetasol propionate, co-trimoxazole, colfosceril palmitate, dextroamphetamie sulfate, digoxin, enalapril maleate, epoprostenol, esomepraxole magnesium, fluticasone propionate, furosemide, hydrochlorothiazide, hydrochlorothiazide/triamterene, lamivudine, lamotrigine, lithium carbonate, losartan potassium, melphalan, mercaptopurine, mesalazine, mupirocin calcium cream, nabumetone, naratriptan, omeprazole, ondansetron hydrochloride, ovine, oxiconazole nitrate, paroxetine hydrochloride, prochlorperazine, procyclidine hydrochloride, pyrimethamine, ranitidine bismuth citrate, ranitidine hydrochloride, rofecoxib, ropinirole hydrochloride, rosiglitazone maleate, salmeterol xinafoate, salmeterol, fluticasone propionate, sterile ticarcillin disodium/clavulanate potassium, simvastatin, spironolactone, succinylcholine chloride, sumatriptan, thioguanine, tirofiban HCl, topotecan hydrochloride, tranylcypromine sulfate, trifluoperazine hydrochloride, valacyclovir hydrochloride, vinorelbine, zanamivir, zidovudine, zidovudine or lamivudine, or mixtures thereof.

### II. Introduction

Fluorescent dyes can be associated with, such as entrapped in, nanoparticles. Without being limited to a particular theory of operation, it may be that the nanoparticle protects the dye molecules from direct exposure to environmental oxygen, and thus greatly enhances the photostability of the entrapped dye. Furthermore, because a large number of dye molecules can be associated with or entrapped inside a nanoparticle, high fluorescence emission can be obtained. The fluorescence emission can exceed that of the dye molecule itself or even the intensity of quantum dots. Nanoparticles also can be selected that have little toxicity and are biocompatible. Nanoparticles suitable for disclosed embodiments of the present invention can be purchased, or are readily prepared from, inexpensive starting materials following simple synthetic procedures.

Once nanoparticles comprising signal generating moieties are obtained, such as fluorescent dyes, they are then coupled to interesting molecules, such as biological molecules or therapeutic agents, which may be referred to herein as ligands. Ligand labeling often requires robust conjugation where the labeling agent is covalently attached to the ligand. For many biomolecules, this can be conveniently accomplished, for example, by using commercial kits of chemically-derivatized labeling agents. For ligands that lack functional groups or are difficult to derivatize, the labeling can be complex or difficult to achieve.

Certain disclosed embodiments of the present invention concern a highly efficient method for coupling ligands to biological molecules or therapeutic agents using insertion reactions, such as the C—H insertion reaction of perhalophenylazides (PFPA). Examples in addition to those provided herein of particular PHPAs, and how to make and use PHPAs, can be found in U.S. Pat. Nos. 5,580,697, 5,582,955, 5,587,273, 5,830,539, 6,022,597, each of which is incorporated herein by reference. This coupling chemistry can conjugate a variety of molecules regardless of their chemical structures or the nature of their functional group.

PHPA-functionalized nanoparticles comprising signal generating moieties provide highly efficient labels for carbohydrate ligands. Carbohydrates are an important class of biomolecules involved in many important biorecognition processes, including for example cell communication, immune responses, fertilization, and infections. Studies of these processes are however hampered by the high complexity of glycan structures and the lack of efficient bioanalytical tools. Disclosed embodiments address some of these challenges by efficiently labeling underivatized carbohydrates. Labeled carbohydrates can be used to detect lectins, and can be used to evaluate biological processes involving lectins. Glyco-nanoparticle-signal generating moiety conjugates also can be used to image infectious agents, such as bacteria, and to probe carbohydrate-protein interactions on a lectin microarray.

### III. Perhalophenylazide-Derived Nanoparticle Probes

Particular embodiments concern perhalophenylazide-derived (PHPA-derived) nanoparticle probes comprising a nanoparticle, a perhalophenylazide (PHPA), and a molecular probe.

A. Nanoparticles

A person of ordinary skill in the art will appreciate that particular embodiments can be used with any nanoparticle, or combinations of nanoparticles, now known or hereafter developed that is useful for the methodologies disclosed herein. Further guidance concerning appropriate selection of a nanoparticle for a particular application can be based on additional factors, such as toxicity and biocompatibility; availability; complexity of synthetic procedures used to make the nanoparticles; cost; etc.

Particularly useful classes of nanoparticles can be metalloids and metal oxides. Examples of suitable metalloid and metal oxide nanoparticles include, without limitation, silica (SiO₂) nanoparticles, titania (Tio₂) nanoparticles, zinc oxide nanoparticles (ZnO) and yttrium vanadium oxide (YVO₄) nanoparticles. Metals define another class of useful nanoparticles, such as gold nanoparticles and silver nanoparticles. Additional examples of nanoparticles include lanthanum phosphates (LaPO₄), and organic nanoparticles, such as polystyrene nanoparticles and graphene nanostructures.

In particular embodiments of the disclosed nanoparticle probes, the size of the nanoparticle can be manipulated. For example, the size of the nanoparticle may control the number of ligands that can be accommodated on the nanoparticle and the distance between the ligands. In certain embodiments, smaller glyco nanoparticles typically have higher binding affinities with biological molecules, such as lectins, than larger glycol nanoparticles. The size of nanoparticles formed can be conveniently controlled by varying the reagent concentrations during nanoparticle preparation. Monodisperse nanoparticles with diameters ranging from about 1 to at least about 200 nm can be obtained efficiently using techniques known to a person of ordinary skill in the art. In particular embodiments, the nanoparticle diameter ranges from about 5 to about 100 nm. Certain embodiments concern nanoparticles having a diameter ranging from about 5 to about 30 nm and further certain embodiments concern nanoparticles having a diameter ranging from about 20 to about 100 nm.

B. Signal Generating Moieties

One aspect of the present invention concerns forming nanoparticles that include signal generating moieties that can be coupled to biological molecules or therapeutic agents. While virtually any signal generating moiety useful for biological applications can be associated with, conjugated to, or entrapped in, nanoparticles according to disclosed embodiments, the invention is usefully exemplified with reference to entrapping fluorescent dyes inside nanoparticles. This has been accomplished in working examples by formation of nanoparticles from nanoparticle starting materials, such as monomers that can be polymerized to form nanoparticles, wherein the starting materials also include fluorescent dyes.

In particular working embodiments, the nanoparticle of the nanoparticle probe can further comprise a signal generating moiety. While virtually any signal generating moiety useful for biological applications can be associated with, conjugated to, or entrapped in the disclosed nanoparticles, particular embodiments concern entrapping fluorescent dyes inside nanoparticles.

Examples of fluorescent dyes include fluorescein and fluorescein isothiocyanate (a reactive derivative of fluorescein), which have the structures illustrated below.

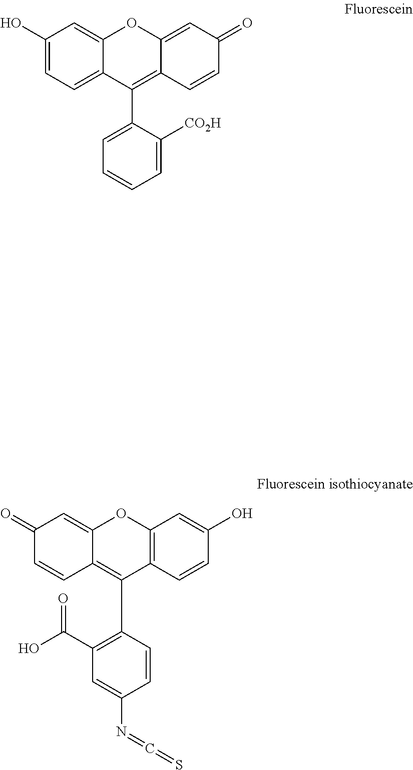 [SMILES: O=C(O)c1ccccc1-c1c2ccc(=O)cc-2oc2cc(O)ccc12 ; O=C(O)c1cc(N=C=S)ccc1-c1c2ccc(=O)cc-2oc2cc(O)ccc12]

Flourescein and fluorescein isothiocyanate are common fluorophores that can be chemically attached to other, non-fluorescent molecules to create new fluorescent molecules for a variety of applications. Other historically common fluorophores are rhodamine, derivatives of rhodamine (e.g. tetramethyl rhodamine isothiocyanate, TRITC), coumarin, and cyanine, some of which are illustrated below.

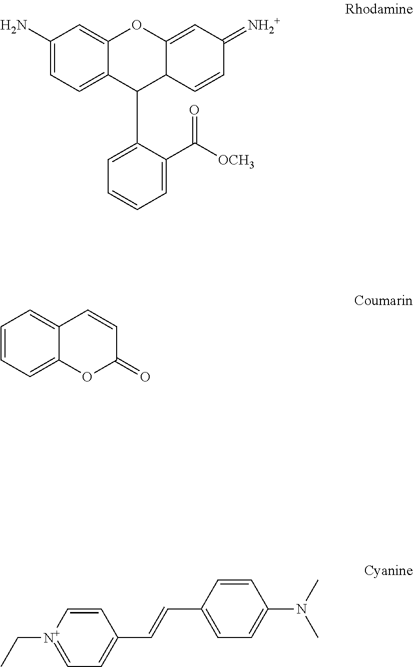 [SMILES: COC(=O)c1ccccc1C1c2ccc(N)cc2OC2=CC(=[NH2+])C=CC21 ; O=c1ccc2ccccc2o1 ; CC[n+]1ccc(/C=C/c2ccc(N(C)C)cc2)cc1]

While the above described fluorophores are commonly used in biology and diagnostic applications, newer generations of fluorophores suitable for the present nanoparticle probes often are more photostable, brighter, and/or less pH-sensitive than traditional dyes. Moreover, various additional classes of fluorophores suitable for the present nanoparticle probes are known, including without limitation: quantum dots (2-10 nm in diameter, 100-100,000 atoms) and proteins, such as green fluorescent proteins (GFP). Common dye families include, but are not limited to: Xanthene derivatives, such as fluorescein, rhodamine, Oregon green, eosin, Texas red, and Cal Fluor dyes; Cyanine derivatives, such as indocarbocyanine, oxacarbocyanine, thiacarbocyanine, merocyanine, and Quasar dyes; Naphthalene derivatives, such as dansyl and prodan derivatives; Coumarin derivatives; oxadiazole derivatives, such as pyridyloxazole, nitrobenzoxadiazole and benzoxadiazole; Pyrene derivatives, such as cascade blue, etc.; Oxazine derivatives, such as Nile red, Nile blue, cresyl violet, oxazine 170, etc.; Acridine derivatives, such as proflavin, acridine orange, acridine yellow, etc.; Arylmethine derivatives, such as auramine, crystal violet, and malachite green; and Tetrapyrrole derivatives, such as porphyrin, phtalocyanine, and bilirubin.

Various dyes also are commercially available, including: CF dye (Biotium), BODIPY (Invitrogen), Alexa Fluor (Invitrogen), DyLight Fluor (Thermo Scientific, Pierce), Atto and Tracy (Sigma Aldrich), FluoProbes, (Interchim). Specific examples of such dyes include, without limitation, those listed in Table 1.

TABLE 1
Exemplary Dyes

| Dye | Ex^{a} (nm) | Em^{b} (nm) | MW^{c} |
|---|---|---|---|
| Hydroxycoumarin | 325 | 386 | 331 |
| Aminocoumarin | 350 | 445 | 330 |
| Methoxycoumarin | 360 | 410 | 317 |
| Cascade Blue | (375); 401 | 423 | 596 |
| Pacific Blue | 403 | 455 | 406 |
| Pacific Orange | 403 | 551 | |
| Lucifer yellow | 425 | 528 | |
| NBD | 466 | 539 | 294 |
| R-Phycoerythrin (PE) | 480; 565 | 578 | 240 k |
| PE-Cy5 conjugates | 480; 565; 650 | 670 | |
| PE-Cy7 conjugates | 480; 565; 743 | 767 | |
| Red 613 | 480; 565 | 613 | |
| PerCP | 490 | 675 | |
| TruRed | 490,675 | 695 | |
| FluorX | 494 | 520 | 587 |
| Fluorescein | 495 | 519 | 389 |
| BODIPY-FL | 503 | 512 | |
| TRITC | 547 | 572 | 444 |
| X-Rhodamine | 570 | 576 | 548 |
| Lissamine Rhodamine B | 570 | 590 | |
| Texas Red | 589 | 615 | 625 |
| Allophycocyanin (APC) | 650 | 660 | 104 k |
| APC-Cy7 conjugates | 650; 755 | 767 | |
| Cy2 | 489 | 506 | 714 |
| Cy3 | (512); 550 | 570 | 767 |
| Cy3B | 558 | 572; (620) | 658 |
| Cy3.5 | 581 | 594; (640) | 1102 |
| Cy5 | (625); 650 | 670 | 792 |
| Cy5.5 | 675 | 694 | 1128 |
| Cy7 | 743 | 767 | 818 |
| Hoechst 33342 | 343 | 483 | 616 |
| DAPI | 345 | 455 | |
| Hoechst 33258 | 345 | 478 | 624 |
| SYTOX Blue | 431 | 480 | ~400 |
| Chromomycin A3 | 445 | 575 | |
| Mithramycin | 445 | 575 | |
| YOYO-1 | 491 | 509 | 1271 |
| Ethidium Bromide | 493 | 620 | 394 |
| Acridine Orange | 503 | 530/640 | |
| SYTOX Green | 504 | 523 | ~600 |
| TOTO-1, TO-PRO-1 | 509 | 533 | |
| TO-PRO: Cyanine | | | |
| Monomer | | | |
| Thiazole Orange | 510 | 530 | |
| Propidium Iodide (PI) | 536 | 617 | 668.4 |
| LDS 751 | 543; 590 | 712; 607 | 472 |
| 7-AAD | 546 | 647 | |
| SYTOX Orange | 547 | 570 | ~500 |
| TOTO-3, TO-PRO-3 | 642 | 661 | |
| DRAQ5 | 647 | 681,697 | 413 |
| Indo-1 | 361/330 | 490/405 | 1010 |
| Fluo-3 | 506 | 526 | 855 |
| DCFH | 505 | 535 | 529 |
| DHR | 505 | 534 | 346 |
| SNARF | 548/579 | 587/635 | |

^{a}Excitation wavelength in nanometers.
^{b}Emission wavelength in nanometers.
^{c}Molecular weight.

Quantum dots are another example of a class of a suitable signal generating moieties. Quantum dots have, for example, been constructed of semiconductor materials (e.g., cadmium selenide and lead sulfide) and from crystallites (grown via molecular beam epitaxy). Other quantum dots include, without limitation and solely by way of example, alloy quantum dots such as ZnSSe, ZnSeTe, ZnSTe, CdSSe, CdSeTe, ScSTe, HgSSe, HgSeTe, HgSTe, ZnCdS, ZnCdSe, ZnCdTe, ZnHgS, ZnHgSe, ZnHgTe, CdHgS, CdHgSe, CdHgTe, ZnCdSSe, ZnHgSSe, ZnCdSeTe, ZnHgSeTe, CdHgSSe, CdHgSeTe, InGaAs, GaAlAs, and InGaN.

C. Perhalophenylazide (PHPA) Moieties

According to certain disclosed embodiments, a nanoparticle and/or a signal generating moiety-doped nanoparticle is covalently bonded to a reagent, such as a perhalophenylazide (PHPA), that allows the nanoparticle and/or the signal generating moiety-doped nanoparticle to be coupled to a biological molecule. Covalent bonding is achieved in particular embodiments by conversion of nitrenogenic groups of the PHPA moiety to a nitrene intermediate, which exhibits a high reactivity with the biological molecule, using a reaction-energy source, such as electromagnetic energy, particularly light. The reagent is preferably selected from a group consisting generally of: aryl azides, aliphatic azides, including alkyl azides, alkenyl azides, alkynyl azides, acyl azides, and azidoacetyl derivatives, all of which are capable of carrying a variety of substituents. Halogen atoms are present, often as many as may be accommodated, in multiple positions on the reagent molecule adjacent the azide group. Particular embodiments concern using fluorine and/or chlorine atoms, but a person of ordinary skill in the art will recognize that all halogen atoms (iodine, bromine, chlorine, and fluorine) are contemplated by disclosed embodiments.

Disclosed embodiments of PHPAs have the formula illustrated below.

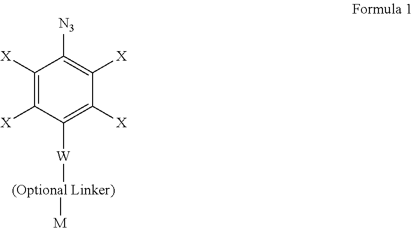

With reference to Formula 1, X may be a halogen; W may be aliphatic, aryl, and a heteroatom-containing moiety; the optional linker may be selected from aliphatic, aryl, and a heteroatom-containing moiety; and M may be selected a metal-, metalloid-, and non-metal-containing moiety. In particular embodiments, X may be selected from iodine, bromine, chlorine, fluorine, and combinations thereof; particularly fluorine and chlorine. In particular embodiments, W may be a hetereoatom-containing moiety selected from, but not limited to, the following: heteroaryl, carbonate (—OC(O)OR^{a}), ether (—OR^{a}), ester (—C(O)OR^{a}), ketone (—C(O)R^{a}), peroxy (—OOR^{a}), phosphine (—PR^{a}R^{b}R^{c}), sulfinyl (—S(O)R^{a}), sulfonyl (—SO₂R^{a}), carbonothioyl (—C(S)R^{a}), oxazole, oxadiazole, imidazole, triazole, tetrazole, amide (—C(O)NR^{a}R^{b}), azo (—NNR^{a}), imide (—C(O)NR^{a}C(O)R^{b}), isonitrile (—NC), amine (—NHR^{a}, —NR^{a}R^{b}), —N-maleimido, —NH-biotinyl, and —CONH-A-S—S—B—NH-biotinyl (where A and B are spacer atoms and the S—S bond is reductively cleaved at a later stage), wherein R^{a}, R^{b}, and R^{c} individually may be selected from hydrogen, aliphatic, aryl, a heteroatom-containing moiety, and any combination thereof. Particular embodiments of optional linkers are discussed subsequently. In particular embodiments, M may be a metal-containing moiety comprising, for example, titanium, zirconium, and zinc. In other embodiments, M may be a metalloid-containing moiety comprising, for example, silicon and boron; and in yet other embodiments, M may be a non-metal-containing moiety comprising, for example, phosphorus, sulfur, and selenium. Certain embodiments concern PHPA moieties wherein M may be selected from silyl, silyl ether, titanyl, titanyl ether, phosphate (—OP(O)(OH)₂), phosphoryl (—P(O)(OH)₂), phosphine (—PR^{a}R^{b}R^{c}), and thiols. Particular disclosed examples are perfluorophenylazide (PFPA) moieties that have a general formula 2, where W, the optional linker, and M are as recited above.

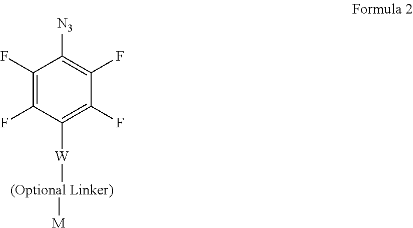

In certain embodiments, the PFPA moiety has any one of the formulas shown below.

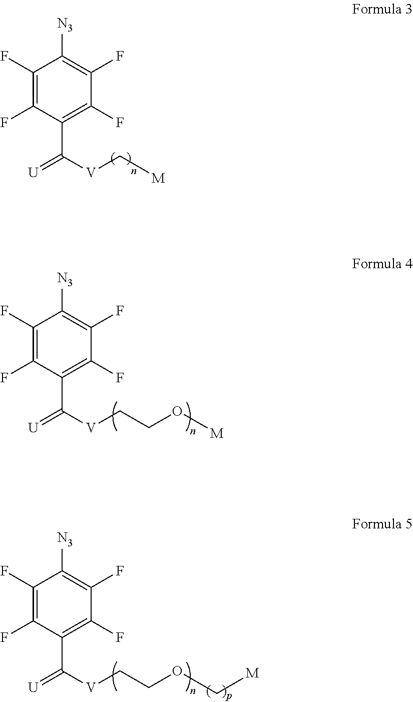

With reference to Formula 3-5, U is selected from O, S, NH, and NR^{a}, where R^{a} may be as previously recited; V is selected from O, S, NH, and NR^{a}, where R^{a} may be as previously recited; M may be selected from a metal-, metalloid-, and non-metal-containing moiety; and n and p individually may range from 0 to about 20; more typically from about 1 to about 10. In particular embodiments, M may be selected from silyl, silyl ether, titanyl, titanyl ether, phosphate (—OP(O)(OH)₂), phosphoryl (—P(O)(OH)₂), phosphine (—PR^{a}R^{b}R^{c}), and thiols. Particular embodiments of the disclosed PFPA moieties are shown below.

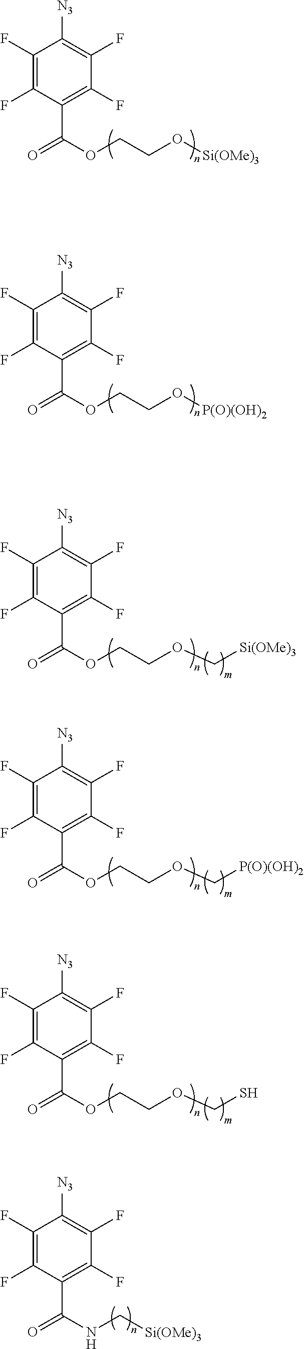

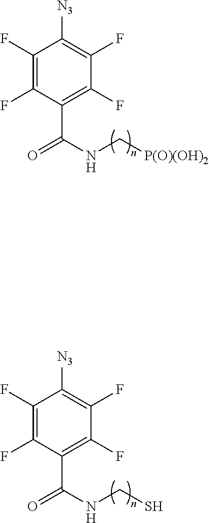

Even more particular working embodiments have the formulas shown below.

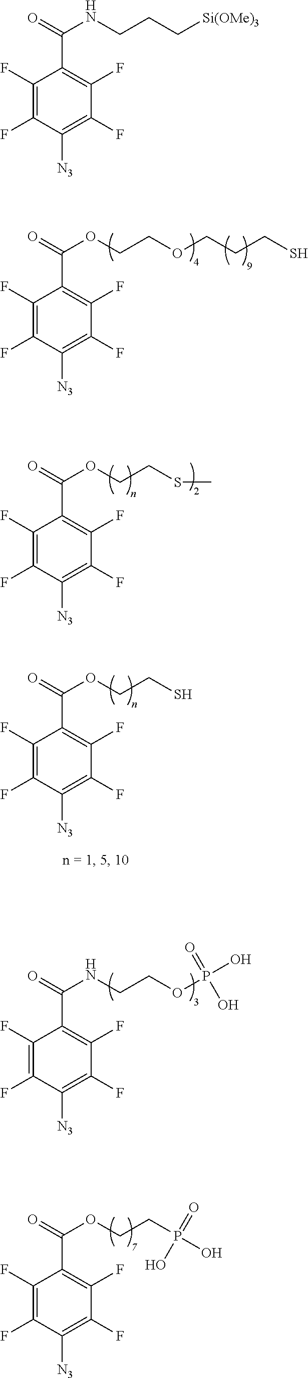

In particular embodiments, the perhalophenylazide moiety may have a formula illustrated below.

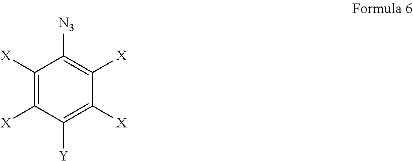

With reference to Formula 6, X may be a halogen, and Y may be selected from a heteroatom-containing moiety capable of undergoing further chemical manipulation to make a perhalophenylazide moiety having, for example, any one of Formulas 1-5, and combinations thereof. In particular embodiments, X may be selected from iodine, bromine, chlorine, fluorine, and combinations thereof; particularly fluorine and chlorine. In particular embodiments, Y may be a heteroatom-containing moiety, wherein the hetereoatom-containing moiety may be selected from, but is not limited to, the following: heteroaryl, halogen, (iodine, bromine, chlorine, and fluorine), aldehyde (—CHO), acyl halide ([—C(O)X], where X may be selected from fluorine, chlorine, bromine, and iodine), carbonate (—OC(O)OR^{a}), carboxyl (—C(O)OH), carboxylate (—COO⁻), ether (—OR^{a}), ester (—C(O)OR^{a}), hydroxyl (—OH), ketone (—C(O)R^{b}), peroxy (—OOR^{b}), hydroperoxy (—OOH), phosphate (—OP(O)(OH)₂), phosphoryl (—P(O)(OH)₂), phosphine (—PR^{a}R^{b}R^{c}), sulfinyl (—S(O)R^{a}), sulfonyl (—SO₂R^{a}), carbonothioyl (—C(S)R^{a} or C(S)H), sulfino (—S(O)OH), sulfo (—SO₂OH), thiocyanate (—SCN), isothiocyanate (—NCS), oxazole, oxadiazole, imidazole, triazole, tetrazole, amide (—C(O)NR^{a}R^{b}), azide (—N₃), azo (—NNR^{a}), cyano (—OCN), isocyanate (—NCO), imide (—C(O)NR^{a}C(O)R^{b}), nitrile (—CN), isonitrile (—NC), nitro (—NO₂), nitroso (—NO), nitromethyl (—CH₂NO₂), amine (—NH₂, —NHR^{a}, —NR^{a}R^{b}), —N-maleimido, —NH-biotinyl, —CONH-A-S—S—B—NH-biotinyl (where A and B are spacer atoms and the S—S bond is reductively cleaved at a later stage) and any homologated derivatives thereof, wherein R^{a}, R^{b}, and R^{c} individually may be selected from hydrogen, aliphatic, aryl, a heteroatom-containing moiety, and any combination thereof. A person of ordinary skill in the art will recognize that certain recitations of Y include functional groups that may be converted, through additional chemical transformations, to a different functional group suitable for subsequent coupling reactions. For example, if Y is a halogen, a carbonylation reaction, such as a stannane-mediate carbonylation or palladium-mediated carbonylation, with a suitable coupling partner may be used to convert the halogen to a carbonyl-containing moiety, such as an aldehyde or an ester.

Particular working embodiments of the present invention used perfluorophenyl azides (PFPAs) having the formula illustrated below.

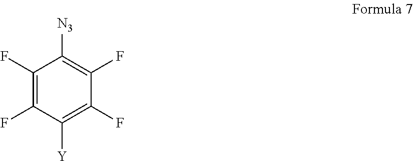

With reference to this general formula, Y is as recited above. In particular working examples, PHPAs may be derived from a 4-azido-2,3,5,6-tetrahalobenzoic acid, illustrated below.

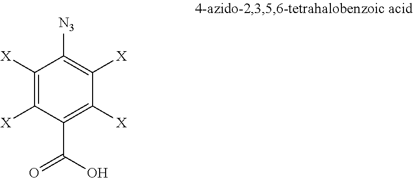

The disclosed perhalophenylazide moieties having a Formula 6 and/or 7 can be further modified using a moiety having a general Formula 8 to make perhalophenylazide moieties having, for example, any one of Formulas 1-5, and combinations thereof.

Z-(Optional Linker)-M Formula 8

With reference to Formula 8, M and the optional linker are both as previously recited in Formulas 1-5; and Z may be a nucleophilic group or an electrophilic group. In particular embodiments, Z may be nucleophilic and may be selected from hydroxyl (—OH), thiol (—SH), amine (—NH₂, —NHR^{a}, —NR^{a}R^{b}), the anions formed from these groups, alkyl lithium moieties (e.g. LiCR^{a}R^{b}R^{c}), metal-containing compounds (e.g. MgCR^{a}R^{b}R^{c} and SnCR^{a}R^{b}R^{c}), and various boronic acids. In other embodiments, Z may be electrophilic and may be selected from aldehyde (—CHO), acyl halide ([—C(O)X], where X may be selected from fluorine, chlorine, bromine, and iodine), carbonate (—OC(O)OR^{a}), carboxyl (—C(O)OH), ester (—C(O)OR^{a}), ketone (—C(O)R^{a}), sulfinyl (—S(O)R^{a}), sulfonyl (—SO₂R^{a}), sulfino (—S(O)OH), sulfo (—SO₂OH), amide (—C(O)NR^{a}R^{b}), cyano (—OCN), isocyanate (—NCO), imide (—C(O)NR^{a}C(O)R^{b}), and nitrile (—CN); and R^{a}, R^{b}, and R^{c} independently may be hydrogen, aliphatic, aryl, a heteroatom-containing moiety and any combination thereof. A person of ordinary skill in the art will recognize that in embodiments where Z is electrophilic, Y may be selected from nucleophilic heteroatom-containing moieties; and when Z is nucleophilic, Y may be selected from electrophilic heteroatom-containing moieties.

Particular working examples include, but are not limited to, 3-aminopropyltimethoxysilane, which is illustrated below.

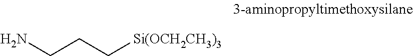

PHPAs also may comprise linkers as previously illustrated (e.g. Formulas 1-5). Linkers of varying length and structure can be used. Linkers can be selected from aliphatic groups, aryl groups, and heteroatom-containing moieties. Particular examples of linkers include, but are not limited to, alkylene glycols, such as poly(ethylene glycol) (PEG), and functionalized glycols, such as 2-[2-(2-chloro-ethoxy)-ethoxy]-ethanol. A suitable linker length can be determined empirically by a person of ordinary skill in the art, such as by using varying linker lengths and plotting the amount of binding versus the linker length. The linker extends the PHPA moiety further away from the surface and allows the molecular probe to more easily contact the PHPA moiety. If the PHPA moiety is too close to the nanoparticle surface, it may be difficult for the molecular probe to interact with and bind to the nanoparticle, particularly if there is any roughness or unevenness to the substrate surface. In particular embodiments, the affinity of molecular probe-labeled nanoparticles for selective targets can increase with the length of the linker. For example, the apparent dissociation constant (K_{d}) value of glycol-labeled (e.g. mannose) gold nanoparticles with a protein (e.g. a lectin, such as Con A), can increase when linkers of varying lengths are used.

Without being limited to a particular theory of operation, it is currently believed that upon photochemical or thermal activation, a PHPA is converted to a singlet perhalophenylnitrene. The perhalophenylnitrene subsequently forms a robust covalent linkage with neighboring molecules via a CH insertion reaction. A particular embodiment of this chemical reactivity is illustrated below in Scheme 1.

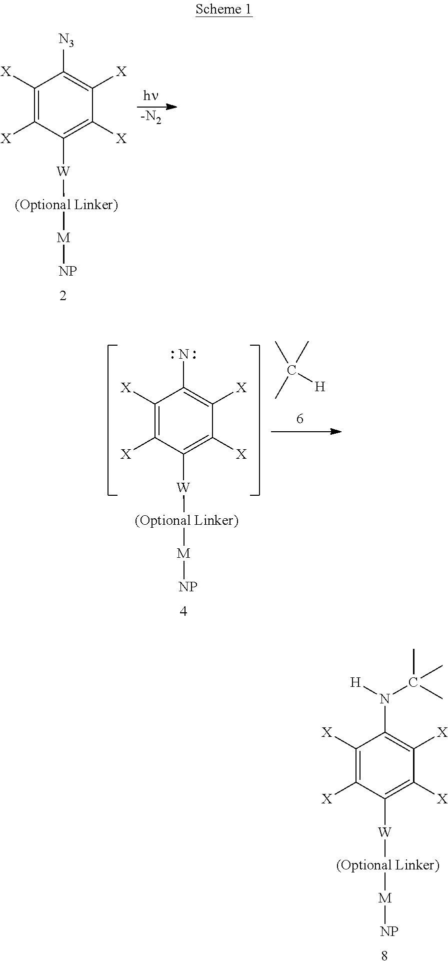

According to Scheme 1, a PHPA compound 2 is exposed to an energy source and is converted to a nitrene intermediate 4. The nitrene intermediate 4 is simultaneously or sequentially reacted with a coupling partner, such as isobutane 6, to produce a functionalized PHPA 8.

A person of ordinary skill in the art will recognize that these examples are only meant as an illustration and are not meant to limit the scope of the disclosed PHPA-derived nanoparticle probes.

D. Molecular Probes

Disclosed embodiments of the present invention are useful for detecting desired molecules for various reasons, including for diagnosing diseases such as cancer, for studying interactions between binding pairs, and for evaluating the target and/or effectiveness of a therapeutic agent. Many biological molecules are difficult to label with signal generating moieties, such as fluorescent molecules. A particular class of biological molecules that are difficult to label are carbohydrates, including monosaccharides, and the polymeric forms of such molecules, including disaccharides, oligosaccharides (a polymer of saccharides where the number of saccharides is relatively small, such as from about 3 to about 10 saccharide monomers), polysaccharides, glycans, etc. These are complex molecules, and the ability to label these molecules with suitable signal generating moieties has proved difficult. While carbohydrates are of particular importance to the presently disclosed methodology, other biological molecules also can be labeled with signal generating moieties according to the embodiments disclosed herein. Accordingly, “biological molecules” can refer to carbohydrates, amino acids, oligomers of amino acids, proteins, nucleic acids, oligomers of nucleic acids, DNA, RNA, lipids, etc.

Certain embodiments of the present invention concern binding labeled signal generating moieties to a binding partner. For example, a sugar or carbohydrate may be used to recognize/bind to a lectin, and the resulting binding pair is then detected, such as by fluorescent imaging. Lectins are sugar-binding proteins highly specific for particular carbohydrates. In this example, either the lectin, or the carbohydrate, can be labeled using PHPAs. One embodiment of a method for conjugating a lectin, such as Con A, to a dye doped nanoparticle, is illustrated schematically below.

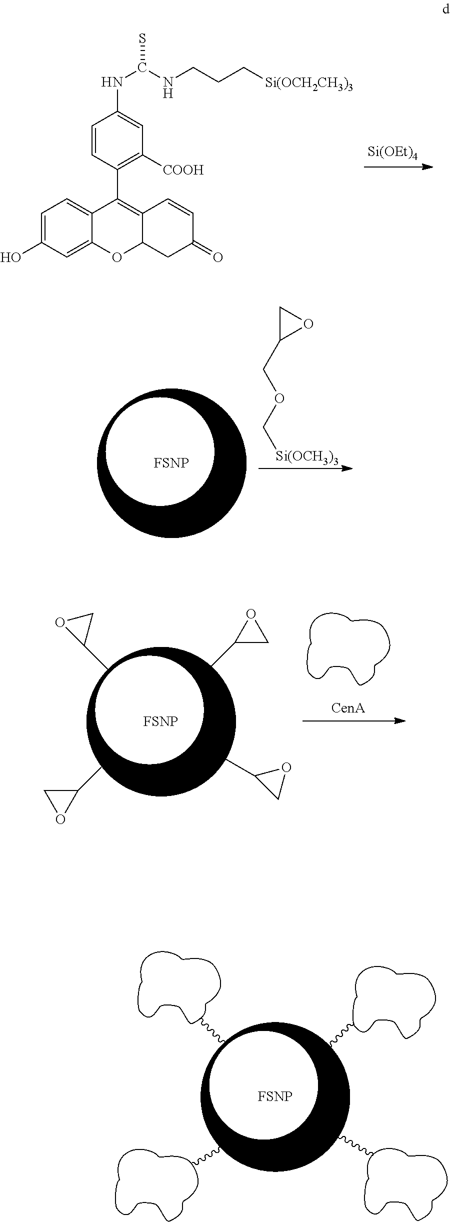

To enhance the signal resulting from a binding event, both the lectin and the sugar or carbohydrate can be labeled according to disclosed embodiments of the present invention. As a result, both members of a particular binding pair are effectively labeled with, for example, a fluorescent dye. A person of ordinary skill in the art will appreciate that the present invention is not limited to labeling just sugars/polymers thereof and lectins, rather that this example simply illustrates the point of labeling both members of a specific binding pair.

Particular embodiments concern using PHPA-derived nanoparticle probes comprising molecular probes. The molecular probe is selected from moieties capable of interacting with a target. The molecular probe can be selected from a biomolecule, such as a carbohydrate, an amino acid, an amino acid oligomer, a protein, a nucleic acid, a nucleic acid oligomer, RNA and DNA; or a therapeutically important compound. In particular embodiments, carbohydrates can be used as a molecular probe. Carbohydrates, (e.g. monosaccharides, disaccharides, polysaccharides, and oligosaccharides), are capable of target-based recognitions, such as, but not limited to, cell-based recognitions. Examples of carbohydrates include, without limitation, Galβ1-4Glc, Glcβ1-4Glc, Glcα1-2Glc, GlcNAcβ1-4GlcNAc, Manα1-2Man, Manα1-2Manα1-2Man, Galα1-3(Fucα1-2)Galβ1-3GlcNAcβ, Neu5Acα2-3Galβ1-4Glcβ, Galβ1-3GalNAc, Fucα1-2Gal-R, GalNAcαSer/Thr, Galα1-3Gal-R, Galβ1-3(Fucα1-4)GlcNAc-R, Galβ1-4(Fucα1-3)GlcNAc-R, GalNAcα1-3GalNAcβ1-R, and 3Galα1-4Galβ1-4Glc-R.

Other embodiments concern using biomolecules, such as nucleotides, which can be coupled to a carbohydrate, such that the biomolecules can be detected as a result of the carbohydrate. See, for example, U.S. Pat. No. 7,220,854, which discloses forming compounds comprising a nucleotide and a sugar residue, such as a triose, tetrose, pantose, hexose, heptose and octose, a monosaccharide, a disaccharide, an oligosaccharide, and a polysaccharide. The sugar or oligo or polymer thereof can also include a signal generating moiety, such as a fluorescent component, such as fluorescein, rhodamine and dansyl. Such sugars can be detected by disclosed methods. The disclosed molecular probes can be acquired from commercial sources or synthesized according to methods known to a person of ordinary skill in the art.

E. PHPA-Derived Nanoparticle Probes/Conjugates

In particular embodiments, the perhalophenylazide-derived nanoparticle probe (as referred to as “conjugate” herein) has a Formula 9, shown below.

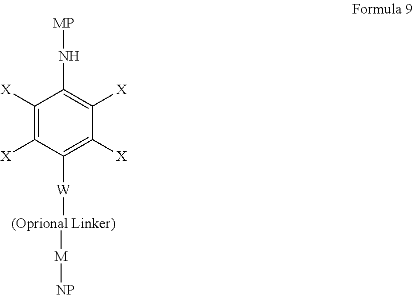

With reference to Formula 9, X may be selected a halogen; MP may be a molecular probe selected from a biomolecule, such as, but not limited to, an antibody, a carbohydrate, an amino acid, an amino acid oligomer, a protein, a nucleic acid, a nucleic acid oligomer, RNA, DNA, a lipid, and combinations thereof; W may be aliphatic, aryl, and a heteroatom-containing moiety; the optional linker may be selected from aliphatic, aryl, and a heteroatom-containing moiety; M may be selected a metal-, metalloid-, and non-metal-containing moiety; and NP may be a nanoparticle selected from a silica nanoparticle, a titania nanoparticle, a zinc oxide nanoparticle, a yttrium vanadium oxide nanoparticle, a gold nanoparticle, a silver nanoparticle, a lanthanum phosphate nanoparticle, a polystyrene nanoparticle, a graphene nanoparticle, and combinations thereof.

In particular embodiments, X may be selected from iodine, bromine, chlorine, fluorine, and combinations thereof; particularly fluorine and chlorine. In particular embodiments, W may be a hetereoatom-containing moiety selected from, but not limited to, the following: heteroaryl, carbonate (—OC(O)OR^{a}), ether (—OR^{a}), ester (—C(O)OR^{a}), ketone (—C(O)R^{a}), peroxy (—OOR^{a}), phosphine (—PR^{a}R^{b}R^{c}), sulfinyl (—S(O)R^{a}), sulfonyl (—SO₂R^{a}), carbonothioyl (—C(S)R^{a}), oxazole, oxadiazole, imidazole, triazole, tetrazole, amide (—C(O)NR^{a}R^{b}), azo (—NNR^{a}), imide (—C(O)NR^{a}C(O)R^{b}), isonitrile (—NC), amine (—NHR^{a}, —NR^{a}R^{b}), —N-maleimido, —NH-biotinyl, and —CONH-A-S—S—B—NH-biotinyl (where A and B are spacer atoms and the S—S bond is reductively cleaved at a later stage), wherein R^{a}, R^{b}, and R^{c} individually may be selected from hydrogen, aliphatic, aryl, a heteroatom-containing moiety, and any combination thereof. Particular embodiments of optional linkers include alkylene glycol linkers, and aliphatic linkers having a formula —(CH₂)_{q}—, where q ranges from about 1 to about 20; more typically from about 1 to about 10. In particular embodiments, M may be a metal-containing moiety comprising, for example, titanium, zirconium, and zinc. In other embodiments, M may be a metalloid-containing moiety comprising, for example, silicon and boron; and in yet other embodiments, M may be a non-metal-containing moiety comprising, for example, phosphorus, sulfur, and selenium. Certain embodiments concern perhalophenylazide moieties wherein M may be selected from silyl, silyl ether, titanyl, titanyl ether, phosphate (—OP(O)(OH)₂), phosphoryl (—P(O)(OH)₂), and phosphine (—PR^{a}R^{b}R^{c}), and thiols.

Even further embodiments concern perfluorophenylazide-derived nanoparticle probes having the general formulas illustrated below.

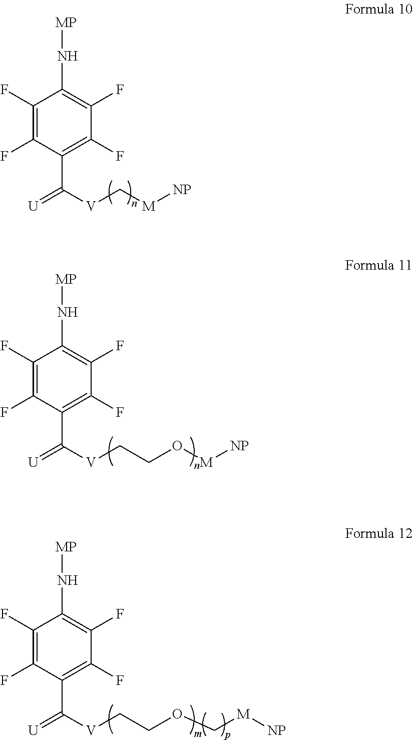

With reference to Formulas 10, 11, and 12, MP and NP may be as recited for Formula 9; and U, V, n, and p may be as recited in Formulas 3, 4, and 5. Particular working embodiments have a Formula 13, illustrated below.

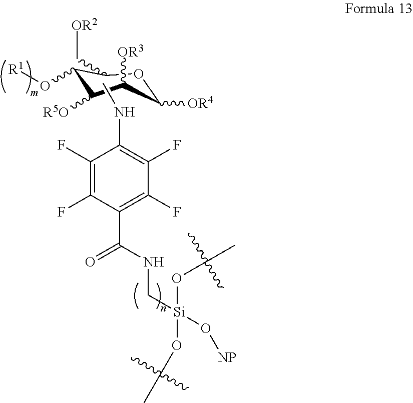

With reference to Formula 13, R¹ may be selected from hydrogen, aliphatic, or a carbohydrate, such as a monosaccharide, a disaccharide, an oligosaccharide, and a polysaccharide; R², R³, R⁴, and R⁵ independently may be selected from hydrogen, aliphatic, aryl, and a heteroatom-containing moiety; and n ranges from zero to about 20, typically 1 to about 10, and more typically 1 to about 5.

### IV. Method of Making Nanoparticle Probes/Conjugates

Particular embodiments concern methods of making the PHPA-derived nanoparticle probes. Nanoparticles functionalized with various molecular probes are contemplated by the disclosed methods. The disclosed methods provide a universal platform for synthesizing large libraries of PHPA-derived nanoparticle probes.

A. Synthesis

A particular aspect of the disclosed method concerns forming nanoparticles that are associated with signal generating moieties, such as nanoparticles that comprise conjugated or entrapped signal generating moieties. Various examples of nanoparticle-entrapped dyes are known, including, for example: Silica (SiO₂) nanoparticles H. Ow, D. R. Larson, M. Srivastava, B. A. Baird, W. W. Webb and U. Wiesner, Nano Lett., 2005, 5, 113-117; Lanthanum Phosphate (LaPO₄), P. Schuetz and F. Caruso, Chem. Mater., 2002, 14, 4509-4516; Titania (TiO₂), S. Jeon and P. V. Braun, Chem. Mater. 2003, 15, 1256-1263; YVO₄, F. Wang, X. Xue, and X. Liu, Angew. Chem. Int. Ed. 2008, 47, 906-909; and polystyrene, Yang, C. S.; Chang, C. H.; Tsai, P. J.; Chen, W. Y.; Tseng, F. G.; Lo, L. W. Anal. Chem. 2004. Each of these references is incorporated herein by reference.

Certain working embodiments of the present application concern forming nanoparticles in the presence of a fluorescent dye, such that the dye becomes entrapped inside the nanoparticles. This process is illustrated schematically below.

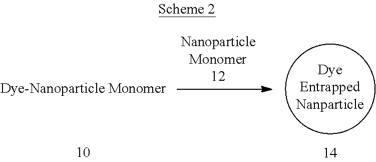

According to Scheme 2, a nanoparticle monomer conjugate 10 comprising a fluorescent dye and a nanoparticle precursor 12, such as a monomer used to form the nanoparticle, are co-polymerized to form the dye entrapped nanoparticle 14. Working embodiments illustrate this approach with reference to making silica nanoparticles, but a person of ordinary skill in the art will recognize that the method can be used with any of the disclosed nanoparticles. The stability of the dye can be substantially increased by trapping it in a nanoparticle.

The nanoparticle and/or signal generating moiety-doped nanoparticle can then be treated with a PHPA moiety. The PHPA moiety is coupled to the nanoparticle and/or the signal generating moiety-doped nanoparticle. A person of ordinary skill in the art will recognize that the term coupling encompasses covalent, ionic, polar-covalent interactions between the PHPA moiety and the nanoparticle, which is may or may not contain a signal generating moiety. In particular embodiments, silica nanoparticles are coupled with PHPA moieties comprising a silane moiety. In other embodiments, gold nanoparticles are coupled with PHPA moieties comprising a thiol moiety. These embodiments are presented as examples of the disclosed method and are not meant to be limiting.

The molecular probe can be coupled to the PHPA-derived nanoparticle using a photochemically initiated CH insertion reaction. In particular embodiments, a reaction mixture comprising a PHPA-derived nanoparticle and a molecular probe is exposed to an energy source, which makes the nanoparticle probe. A particular embodiment of the disclosed method is illustrated below in Scheme 3.

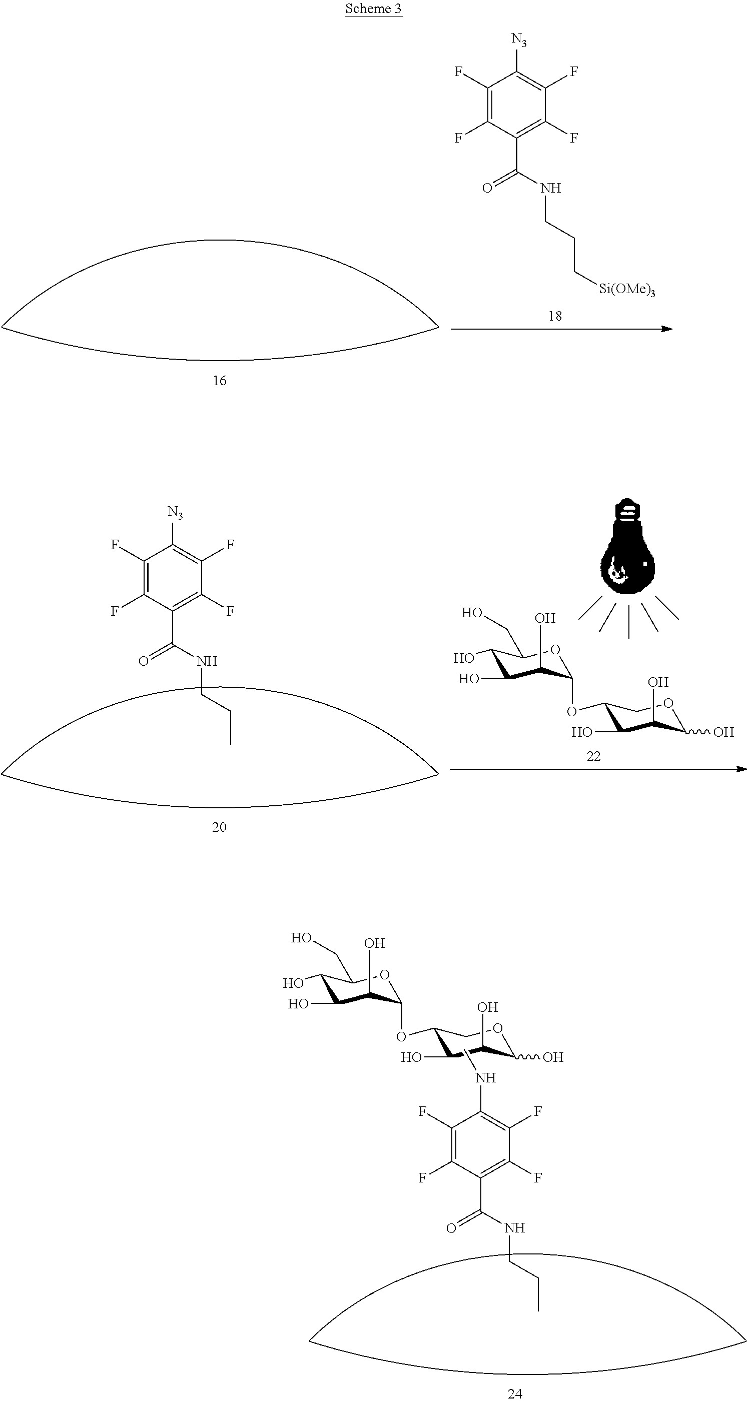

According to Scheme 3, a nanoparticle 16 is coupled with a PHPA moiety 18 to produce a PHPA-derived nanoparticle 20. The PHPA-derived nanoparticle 20 can be coupled with a molecular probe, such as carbohydrate 22, using an energy source to produce the PHPA-derived nanoparticle probe 24. Without being limited to a single theory of operation, it is currently believed that the energy from the energy source converts the PHPA moiety 26 into a perhalophenylnitrene intermediate 28, which can then undergo a CH insertion reaction with the molecular probe 30, to produce the PHPA-derived nanoparticle probe 32, as illustrated in Scheme 4. The disclosed method can result in different binding orientations of the molecular probe to the perhalophenylnitrene intermediate; however, these different binding selectivities do not limit the reactivity of the PHPA-derived nanoparticle probe. The non-selective CH insertion reaction can lead to reduced biased interactions with a target, thereby allowing increased binding motifs of the probe.

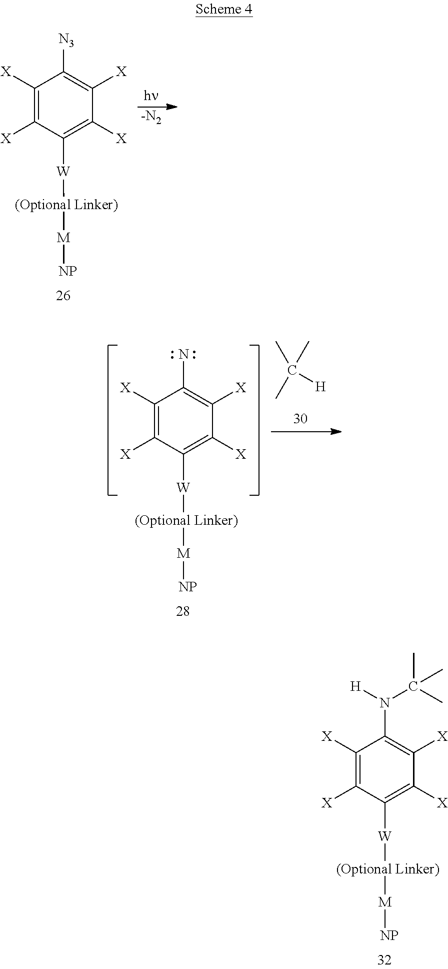

Particular embodiments concern using an energy source to promote the coupling of the molecular probe to the PHPA-derived nanoparticle. The energy source can be any energy source sufficient for promoting nitrene formation, but a person of ordinary skill in the art will recognize that the strength of the energy source must not be so strong as to decompose the molecular probe. Particular embodiments concern using an ultraviolet light as an energy source. Once the desired PHPA-derived nanoparticle probe has been made, excess reagents can be removed by methods known to a person of ordinary skill in the art, such as dialysis, filtration, chromatography, and centrifugation.

The disclosed methods contemplate performing the synthesis by combining all reagents in one mixture and exposing the mixture to an energy source; performing the synthesis by first combining the nanoparticle and the PHPA moiety, followed by sequential addition of the molecular probe and exposure to the energy source; and combinations thereof.

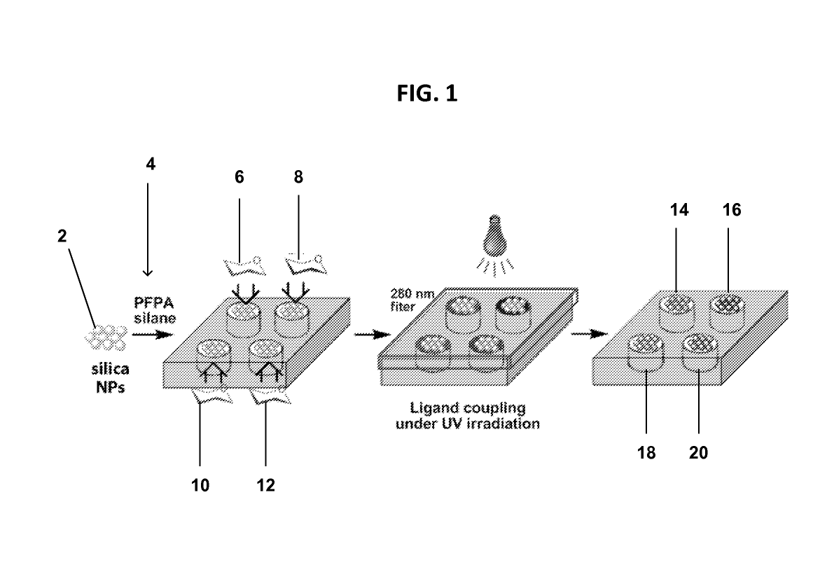

Particular embodiments of the disclosed method concern synthesizing PHPA-derived nanoparticle probe libraries using a high-throughput protocol. This protocol concerns performing the photocoupling step in parallel under controlled conditions. For example, multiple different reaction wells independently can contain a combination of different or similar PHPA-functionalized nanoparticles and different or similar molecular probes. These multiple different reaction wells are then photoactivated simultaneously with an energy source. Multiple different molecular probes can be employed in each different reaction well. The number of reaction wells can be increased by methods known to a person of ordinary skill in the art, such as by using microarray technology. FIG. 1 illustrates a particular embodiment where a combination of silica nanoparticles 2, PFPA-silane moieties 4, and four different molecular probes, such as glycans 6, 8, 10, and 12, are sequentially placed in four different reaction wells. The reaction wells are then irradiated with an energy source to couple the molecular probes to the perhalophenylnitrene-containing nanoparticles. Multiple different reaction wells 14, 16, 18, and 20 comprising multiple different PHPA-derived nanoparticle probes are then obtained.

B. Nanoparticle Probe/Conjugate Modification

Particular embodiments concern methods for achieving PHPA-derived nanoparticle probe diversity. These methods include varying molecular probe, varying the molecular probe density, varying the linker length and structure, and varying the size of the nanoparticle's diameter.

In particular embodiments, the type of molecular probe can be varied in order to make a diverse number of PHPA probes having different or the same molecular probe(s). By making a variety of PHPA-derived nanoparticle probes, complex targets can be differentiated and classified based on interaction with a variety of PHPA-derived nanoparticle probes, rather than requiring a particular single receptor-probe interaction.

In certain embodiments, variation of the molecular probe density on the nanoparticle can influence the affinity of the PHPA-derived nanoparticle probes for a target. Particular embodiments concern controlling the density of the molecular probe by making nanoparticles functionalized with a combination of photoreactive PHPA moieties and non-photoreactive compounds. Examples of non-photoreactive compounds contemplated by the disclosed method include non-photoreactive thiols (e.g. aliphatic thiols, including alkyl thiols, such as 1-hexanethiol), non-photoreactive silanes, and combinations thereof. In a particular embodiment, 1-hexanethiol was added to PFPA-thiol at a ratio of 9:1 (thiol:PFPA-thiol) to functionalize gold nanoparticles. The number of D-mannose moieties (Man) immobilized on the gold nanoparticles (GNPs) decreased from 3,991 to 107, and the apparent K_{d} value of Man-GNPs with Concanavalin A (Con A, a Man-binding lectin), changed from 0.43 nM to 27.4 nM.

In particular embodiments, the length and structure of the linker can be varied. The affinity of PHPA-derived nanoparticle probes to a target can increase with the length of the linker. In a particular embodiment of the disclosed method, the apparent K_{d} value of Man-GNPs with Con A was 0.43 nM when the PFPA-thiol, Compound 1 was used; 4.0 nM when the PFPA-thiol, Compound 2 was used; 15 nM when the PFPA-thiol, Compound 3 was used; and 19 nM when the PFPA-thiol, Compound 4 was used.

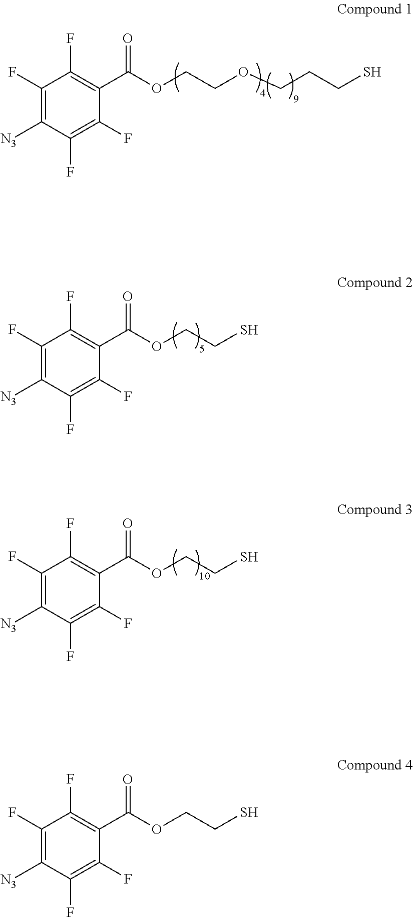

In particular embodiments, the size of the nanoparticle diameter can be varied. The size of the nanoparticle may control the number of ligands that can be accommodated and the distance between them. Smaller PHPA-derived nanoparticle probes typically have higher binding affinities with targets than larger PHPA-derived nanoparticle probes. The size of nanoparticles formed can be conveniently controlled by varying the reagent concentrations during the nanoparticle preparation. Thus, monodisperse nanoparticles with diameters ranging from about 1 to about 200 nm can be obtained efficiently using techniques known to a person of ordinary skill in the art. In particular embodiments, the nanoparticle diameter ranges from about 5 to about 100 nm. Certain embodiments concern nanoparticles having a diameter ranging from about 5 to about 30 nm and further certain embodiments concern nanoparticles having a diameter ranging from about 20 to about 100 nm. Particular embodiments concern using either one of these variations, or combinations thereof to achieve making nanoparticle probes comprising varying numbers of ligands.

### V. Method of Using Nanoparticle Probes/Conjugates

Particular disclosed embodiments concern using PHPA-derived nanoparticle probes to detect, classify, and/or differentiate a target in a sample. In particular embodiments, the disclosed method can be used for the diagnosis, detection, and/or prognosis of a disease. The disclosed method of using the PHPA-derived nanoparticle probes can be used with targets that interact specifically or non-specifically with the probes.

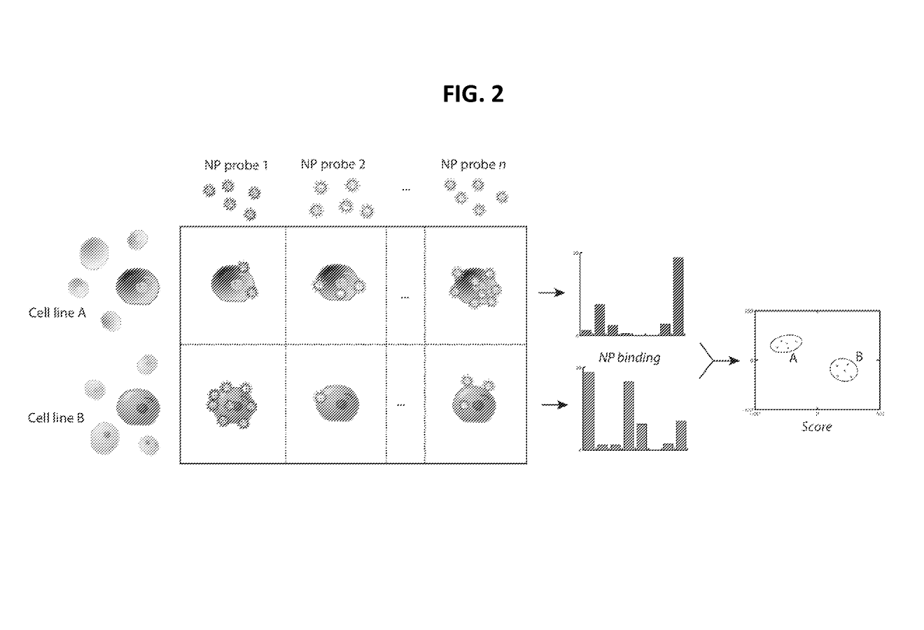

FIG. 2 is a schematic drawing illustrating one embodiment of the disclosed method. With reference to FIG. 2, two different targets 22 and 24 (e.g. Cell line A and Cell line B) can interact with one or more PHPA-derived nanoparticle probes 26, 28, and 30. The interaction between the target and the probe can then be detected by any method capable of detecting the interaction, such as colormetric methods, fluorometric methods, and combinations thereof; and the data can be converted to graphs 32 and 34. The data illustrated in graphs 32 and 34 can be converted to a score plot 36 using statistical analysis. The score plot represents a response pattern for each target, which can be compared and used to classify and differentiate different targets. Particular aspects of the disclosed method are discussed below.

A. Targets

The disclosed method can be used with a variety of targets. The disclosed method can be used to detect, classify, and/or differentiate a single target or multiple different targets. Particular embodiments of targets include, but are not limited to, cell lines, proteins, nucleic acids, and combinations thereof. Examples of cell lines contemplated by the disclosed method include, but are not limited to, cell lines in a normal state, cancer state, malignant state, chemosensitive state, and combinations thereof. Target cell lines can have varying levels of malignancy, aggressiveness, and sensitivity to standard chemotherapy. By way of example, particular cells lines include prostate cell lines, breast cancer cell lines, and pancreatic cell lines. The disclosed method can be used to determine, diagnose, or detect the level of malignancy or sensitivity to chemotherapy of the particular cell line. In addition, the disclosed method can be used with attached cells, detached cells, such as in a cell suspension, and combinations thereof.

In particular embodiments, a target can be selected from a protein, nucleic acid, or combination thereof. A person of ordinary skill in the art will recognize that when reference is made to a target protein it is understood that the nucleic acid sequences associated with that protein can also be used as a target. In particular embodiments, the target is a protein or nucleic acid molecule from a pathogen, such as a virus, bacteria, or intracellular parasite, such as from a viral genome. In other embodiments, a target protein may be produced from a target nucleic acid sequence associated with a disease.

A target nucleic acid sequence can vary substantially in size. Without limitation, the nucleic acid sequence can have a variable number of nucleic acid residues ranging from at least 5 nucleic acid residues, more typically at least 10 nucleic acid residues, or at least about 20, 30, 50, 100, 150, 500, up to at least 1000 residues. Target polypeptides can vary substantially in size, such as including at least one epitope that binds to a peptide specific antibody, or fragment thereof. In some embodiments, the polypeptide can include at least two epitopes that bind to a peptide specific antibody, or fragment thereof.

In specific, non-limiting examples, a target protein is produced by a target nucleic acid associated with a neoplasm, such as a cancer. Numerous chromosome abnormalities, such as translocations, or amplification or deletion, have been identified in neoplastic cells, especially in cancer cells, such as B cell and T cell leukemias, lymphomas, breast cancer, colon cancer, neurological cancers and the like. Therefore, in particular embodiments of the disclosed method, at least a portion of the target molecule is produced by a nucleic acid sequence that has been amplified or deleted in at least a subset of cells in a sample. In one example, the genomic target nucleic acid sequence is selected to include a gene (e.g., an oncogene) that is reduplicated in one or more malignancies (e.g., a human malignancy). For example, HER2, also known as c-erbB2 or HER2/neu, is a gene that plays a role in the regulation of cell growth. The HER2 gene codes for a 185 kd transmembrane cell surface receptor that is a member of the tyrosine kinase family. HER2 is amplified in human breast, ovarian, and other cancers, thus, a HER2 gene can be used as a genomic target nucleic acid sequence.

In other examples, a target protein produced from a nucleic acid sequence is selected to comprise a tumor suppressor gene that is deleted in malignant cells. For example, the p16 region located on chromosome 9p21 is deleted in certain bladder cancers. Chromosomal deletions involving the distal region of the short arm of chromosome 1 and the pericentromeric region of chromosome 19 are examples of characteristic molecular features of certain types of solid tumors of the central nervous system. The aforementioned examples are provided solely for purpose of illustration and are not intended to be limiting.

In other examples, a target protein is selected from a virus or other microorganism associated with a disease or condition. Detection of the virus- or microorganism-derived target nucleic acid sequence (e.g., genomic target nucleic acid sequence) in a cell or tissue sample is indicative of the presence of the organism. For example, the target peptide, polypeptide or protein can be selected from the genome of an oncogenic or pathogenic virus, a bacterium or an intracellular parasite (such as Plasmodium falciparum and other Plasmodium species, Leishmania (sp.), Cryptosporidium parvum, Entamoeba histolytica, and Giardia lamblia, as well as Toxoplasma, Eimeria, Theileria, and Babesia species).

In particular embodiments, the target can be a protein, such as, but not limited to, a lectin. Examples of lectins include, but are not limited to, a mannose binding lectin, a galactose/N-acetylgalactosamine binding lectin, a N-acetylglucosamine binding lectin, a N-acetylneuraminic acid binding lectin, a fucose binding lectin, and combinations thereof. Particular embodiments concern using concanavalin A (Con A), peanut agglutinin (PNA), soybean agglutinin (SBA), and Griffonia simplicifolia (GS-II).

The disclosed method can be used for the detection, diagnosis, and prognosis of disease. In particular embodiments, cells can be analyzed using the disclosed method, which provides a cell surface profile used to evaluate cells. This cell profile can be used to determine the type of disease, the stage of the disease prior to treatment, and the stage of the disease after treatment. Particular embodiments concern using the disclosed method to determine the different stages of development of cancer cells, such as pancreatic, breast, and prostate cancer cells.

B. Interactions Between Perhalophenylazide-Derived Nanoparticle Probes/Conjugates and Targets

Particular embodiments of the disclosed method concern measuring and/or detecting interactions between one or more targets and one or more PHPA-derived nanoparticle probes. These interactions can be specific, non-specific, or combinations thereof. Specific interactions can include those interactions in which a particular PHPA-derived nanoparticle probe has a high affinity for a particular target. For example, particular PHPA-derived nanoparticle probes wherein the molecular probe is a mannose, glucose, galactose, dimannose, diglucose, digalactose, or N-acetylglucosamine have a high affinity for particular lectins, such as ConA, SBA, GS-II, and PNA. Non-specific interactions can include those interactions in which a particular PHPA-derived nanoparticle probe does not have a high affinity for a particular target. For example, particular PHPA-derived nanoparticle probes wherein the molecular probe is lactose, sucrose, arabinose, or cellobiose do not have a high affinity for particular lectins, such as ConA, SBA, and PNA. A person of ordinary skill in the art will recognize that these types of interactions can be detected independently or in combination.

Multiple targets in a single sample also can be detected. For example, a first fluorescent dye or quantum dot could be effectively coupled to a member of a first specific binding pair for detection subsequent to a binding event. A second fluorescent dye or quantum dot, that may emit a wavelength different from that of the first fluorescent dye or quantum dot, could be effectively coupled to a member of a second specific binding pair for detection subsequent to a binding event. Thus, multiple different targets can be detected in a single sample.

C. Detection Techniques

Particular embodiments of the disclosed methods concern differentiating multiple different targets using various detection techniques in order to carry-out pattern recognition. In particular embodiments, at least one target is exposed to at least one PHPA-derived nanoparticle probe. The target will interact with the PHPA-derived nanoparticle probe. The interaction can generate a signal capable of being detected by appropriate detection techniques. For example, the signal can be a colormetric or fluorescent signal. A person of ordinary skill in the art will recognize that the detection technique employed will depend upon the type of PHPA-derived nanoparticle probe used. Particular embodiments concern using detection techniques capable of measuring fluorescence and absorbance. For example, a method using a PHPA-derived nanoparticle probe comprising a fluorescent signal generating moiety can employ a detection technique capable of detecting changes in fluorescence. In particular embodiments, interactions between a target, such as a lectin, and a PHPA-derived silica nanoparticle probe doped with fluorescein can be detected using fluorescence spectroscopy. In other embodiments, a method using a PHPA-derived nanoparticle probe that does not comprise a fluorescent signal generating moiety can employ a detection technique capable of detecting changes in absorbance. In particular embodiments, interactions between a target, such as a lectin, and a PHPA-derived gold nanoparticle probe can be detected using absorbance spectroscopy.

In particular embodiments, the interactions between one or more targets and one or more PHPA-derived nanoparticle probes are detected using fluorescence spectroscopy. Embodiments using fluorescence spectroscopy concern measuring the change in fluorescence intensity between a fluorescein-doped, PHPA-derived nanoparticle probe and a fluorescein-doped, PHPA-derived nanoparticle probe after it has been exposed to a target. Once the PHPA-derived nanoparticle probe has interacted with the target, such as by attaching to the target, any PHPA-derived nanoparticle probes that have not interacted with the target can be removed by methods known to a person of ordinary skill in the art, such as by dialysis, filtration, chromatography, centrifugation, and combinations thereof. The fluorescence intensity of those probes that interact with the target can then be detected. Particular embodiments of the disclosed method concern using fluorescence spectroscopy in conjunction with attached cells, such as cells attached to a well plate of a microarray. Other embodiments concern using fluorescence spectroscopy in conjunction with detached cells (e.g. cell suspensions). In embodiments using detached cells, excess PHPA-derived nanoparticle probes that have not coupled to the cells can be removed using centrifugation. The decrease in the fluorescence is then detected and will correspond to the probes that have been coupled to the singular detached (or multiple detached) cell(s).

In other particular embodiments, the interactions between one or more targets and one or more PHPA-derived nanoparticle probes can be measured using absorbance spectroscopy. Particular embodiments concern using surface plasmon resonance. In these embodiments, both the λₘₐₓ produced before the PHPA-derived nanoparticle probe interacts with the target and the λₘₐₓ produced after the PHPA-derived nanoparticle probe interacts with the target are detected. The change in λₘₐₓ provides a numerical number that can be used in further statistical analysis.

Certain embodiments concern using absorbance spectroscopy to detect interactions between PHPA-derived nanoparticle probes and attached cells. Without being limited to a particular theory of operation, it is currently believed that interactions between the PHPA-derived nanoparticle probes and the attached cells will result in a red-shift (a person of ordinary skill in the art will recognize that a red-shift concerns an increase in wavelength) in the absorption spectrum as the probes couple with the cell surface, thus making a color change. A person of ordinary skill in the art will recognize that the extent of the red-shift, or absorption shift, is directly related to the strength of the interaction between the probe and the cell.

In other embodiments, absorbance spectroscopy can be used to detect interactions between PHPA-derived nanoparticle probes and detached cells. Without being limited to a particular theory of operation, it is currently believed that the interactions between the PHPA-derived nanoparticle probes and the detached cells will result in a decrease in the absorption intensity of the sample solution.

D. Statistical Analysis

Particular embodiments of the disclosed method concern subjecting data obtained from the disclosed detection methods to statistical analysis in order to derive a particular pattern for each detected target. In particular embodiments, statistical methods can be used to find a linear combination of features which characterize and/or differentiate two or more targets. Examples of contemplated statistical methods include Linear Discriminant Analysis (LDA), Principle Cluster Analysis (PCA), and Fisher's linear discriminant analysis. Specific, non-specific, and combinations thereof can be analyzed using the disclosed method.

In particular embodiments, LDA, which uses pattern based recognition, was used to differentiate individual lectins using the number patterns generated by the SPR shift generated in particular embodiments. LDA uses linear combinations of features to distinguish two or more classes of objects (Germain, M. E.; Knapp, M. J. J. Am. Chem. Soc. 2008, 130, 5422-5423). LDA treats the array response as the linear combination of discriminant functions or canonical scores (Wu, Y.; Na, N.; Zhang, S.; Wang, X.; Liu, D.; Zhang, X. Anal. Chem. 2009, 81, 961-966; Miranda, O. R.; You, C.-C.; Phillips, R.; Kim, I.-B.; Ghosh, P. S.; Bunz, U. H. F.; Rotello, V. M. J. Am. Chem. Soc. 2007, 129, 9856-9857). LDA analyzes a training matrix created by a collection of independent and dependent variables, where the dependent variables (e.g. lectins) are classified according to the values generated by the independent variables (e.g. SPR shift). A commercially available software XLSTAT® was used for all discriminant analysis. For all LDA analysis carried out, the tolerance level was 0.001 and the significance level was 0.05. The tolerance level approaches zero when there is little or no significant discrimination between groups. All LDA was done using forward stepwise discriminant analysis. In forward stepwise discriminant function analysis, a model of discrimination is built step-by-step. At each step all variables are reviewed and evaluated to determine which one will contribute most to the discrimination between groups. That variable will then be included in the model, and the process repeats (Equation 1).

Zi=A1X1+A2×2+A3×3+ . . . +AkXk+C Equation 1

With reference to Equation 1, A is the discriminant function, Xi (e.g. X1, X2, X3, etc.) are the discriminating variables, which are the measured values of the independent variables used to predict group membership (e.g. SPR shifts) Ai are discriminant weights (a measure of the discriminating/classifying ability of variable Xi) and C is a constant. When performed in a stepwise manner, variables used to compute the linear functions are selected based on the Wilks' lambda method. Wilks' lambda (Λ) is defined as the ratio of the within-group sum of the squares to the total sum of the squares. Wilks' lamda is a number between 0 and 1 where 0 represents very well separated classes where 1 represents no or little separation.

A person of ordinary skill in the art will recognize that the discriminant analysis can also be applied in predictive classification of cases. For example, once a model has been finalized and the discriminant functions have been derived, predictions can be made to find out where a particular case belongs.

E. Microarrays

Particular disclosed embodiments concern a microarray using the disclosed conjugate. In particular, the disclosed microarray can produce a high-throughput screening strategy for conjugate-target interactions, such as the interactions between a glycan and a lectin, as well as interactions between a cell and the disclosed conjugate. The disclosed microarray provides qualitative and quantitative detection of at least one target. In particular disclosed embodiments, a plurality of targets may be detected, such as at least one target to 100 targets; typically from at least one target to about 50 targets; more typically from at least one target to about 20 targets. In an exemplary working embodiment a microarray was used to detected 14 immobilized lectins. The microarray can also differentiate the specific and non-specific bindings of conjugate-target interactions using fluorescence for visual recognition. Also contemplated by the disclosed microarray is the ability to carry out competition assays to determine binding affinity of embodiments of the disclosed conjugate with at least one target. In particular disclosed embodiments, a plurality of microarrays may be created on a single slide and each microarray may be separated by an isolator, such as a PDMS isolator.

In particular disclosed embodiments, the performance of the microarray, or plural microarrays, was analyzed using the following steps: the conjugate is incubated in a buffer solution within each well on the microarray, each well containing a target; the conjugate is washed with fresh buffer and the slide is dried; the slide is subjected to a microarray scanner and the signal is recorded. Exemplary working embodiments are disclosed herein.

Using the disclosed plural microarrays on one slide provides the following advantages: different assays can be performed simultaneously permitting large-scale glycan profiling; ligand competition assays can be conducted on a single super-microarray to afford the dose response curve, from which the IC₅₀ and apparent K_{D} values can be readily obtained; and high-throughput data outputs are attainable and glycan consumption can be reduced (only ˜3 μg of glycans consumed for each assay).

The high throughput nature of lectin super-microarrays in conjunction with the versatility of nanoparticle-based glycan labeling technique provides a robust and unique approach ideally suited for quantitative glycan profiling in a large library. The protocol could also be applied to other biological entities such as DNA, antibodies, and cells.

### VI. Exemplary Working Embodiments

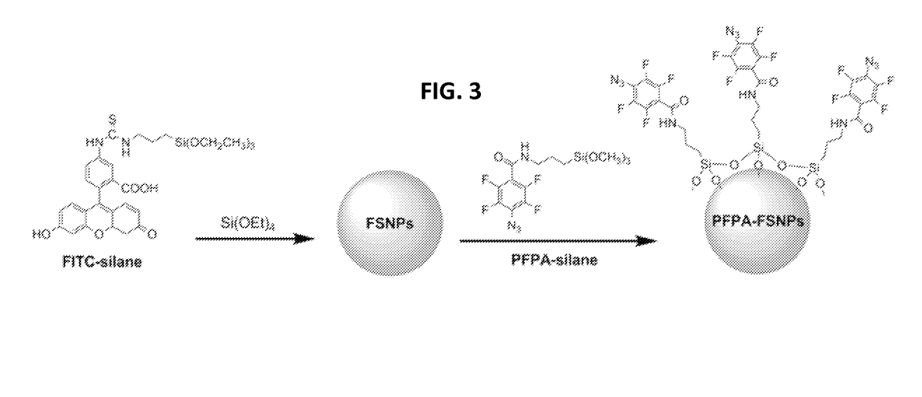

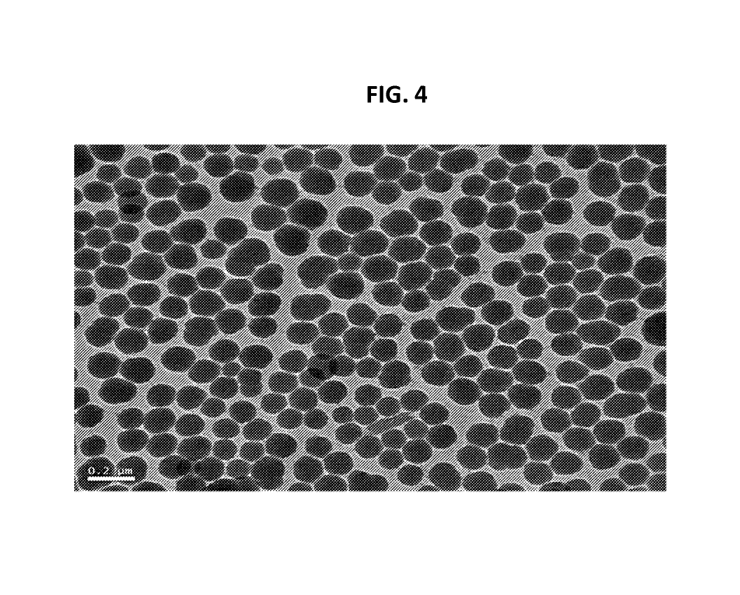

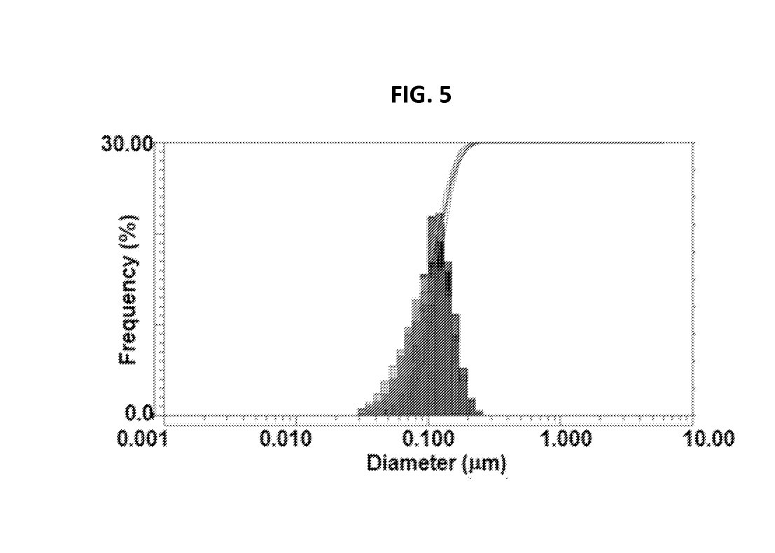

FIG. 3 illustrates a working embodiment used to make exemplary dye-PHPA functionalized nanoparticles. With reference to FIG. 3, fluorescein isothiocyanate (FITC)-doped silica nanoparticles (SNPs) were synthesized using a modified Stöber method. W. Stöber and A. Fink, J. Colloid Interface Sci., 1968, 26, 62-69. The reaction of fluorescein isothiocyanate with 3-aminopropyltriethoxysilane yielded a fluorescein isothiocyanate-silane. The fluorescein isothiocyanate-silane was then copolymerized with tetraethyl orthosilicate. The resulting FSNPs were approximately 100 nm in diameter, showed intense fluorescence even at low particle concentrations (see FIGS. 4 and 5 for particle size characterization).

A person of ordinary skill in the art will appreciate that the selection of the reagents determines the nature of nanoparticles made by this method. For example, the dye can be other than fluorescein. Moreover, appropriate selection of the monomeric material used to make the nanoparticle will result in production of a different nanoparticle.

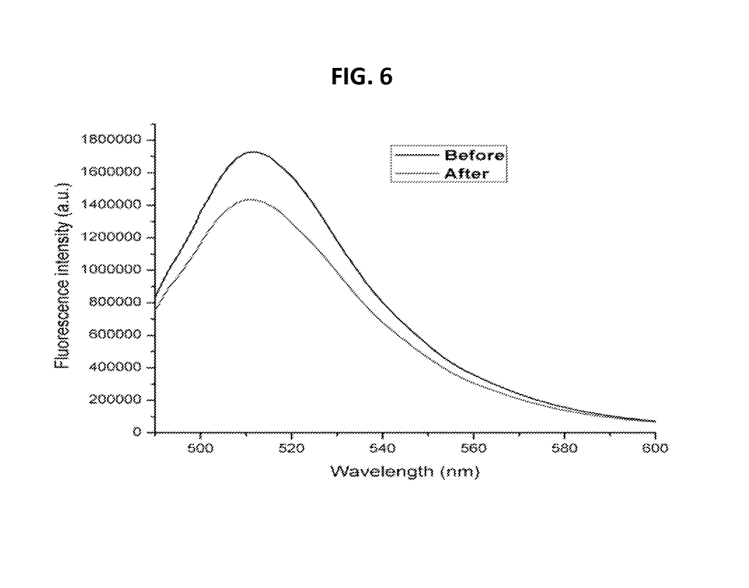

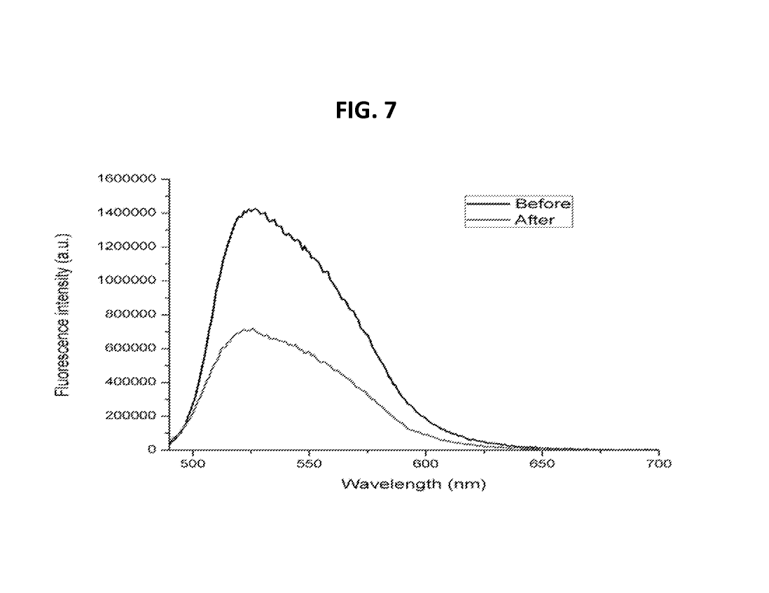

To confirm that the entrapped FITC would withstand the UV irradiation conditions used for a subsequent photocoupling reaction, a solution containing FITC-doped SNPs was irradiated with a 450-W medium-pressure Hg lamp for 10 minutes. The resulting solution remained highly fluorescent, and the fluorescence intensity decreased only to a small extent. See, FIG. 6. On the other hand, when the solution was irradiated under the same conditions, the fluorescent intensity was reduced to about 50% of the original value. See, FIG. 7. This result indicated that the photostability is significantly improved when fluorescent dyes are embedded in nanoparticles, illustrating one of the substantial benefits associated with embodiments of the present invention.

Dye-entrapped nanoparticles were then functionalized with a PHPA. This was accomplished in this working example by treating the as-prepared dye-entrapped nanoparticles with a PHPA-silane. The resulting conjugate can now be conveniently coupled to a desired biomolecule.

Accordingly, a carbohydrate was selected to establish that such complex compounds, which are resistant to other labeling techniques, could be effectively labeled using the method of the present invention. A solution containing a carbohydrate and dye-entrapped nanoparticles was irradiated with a medium-pressure Hg lamp for 10 minutes. Excess reagents were removed by dialysis, and the resulting glycol-dye-entrapped nanoparticles showed excellent water solubility and high fluorescence emission intensity. The density of the probe molecule can be controlled, for instance, by varying the concentration of PHPA. The density of the immobilized carbohydrate was determined using a colorimetric assay, from which the coupling yields were calculated (Table 2, below). The results showed that the photocoupling reaction was highly efficient. The coupling yield, ranging from 36% to 54%, increased with the size of the carbohydrate.

TABLE 2
Ligand density and coupling yield of glyco FSNPs

| | No. of ligands per | No. of ligands | |
|---|---|---|---|
| | FSNP (×10⁴, | per FSNP (×10⁴, | Coupling |
| Carbohydrate ligand | experimental) | calculated) | yield (%) |
| D-Mannose (Man), a | 5.52 | 15.3 | 36 |
| monosaccharide | | | |
| α-1,2-Mannobiose | 2.72 | 5.56 | 49 |
| (Man2), disaccharide | | | |
| α-1,3-1,6-Mannotriose | 2.48 | 4.76 | 52 |
| (Man3), trisaccharide | | | |
| D -Glucose (Glc), | 5.64 | 15.3 | 37 |
| monosaccharide | | | |
| Maltopentose (Glc5), | 1.72 | 3.16 | 54 |
| polysaccharide | | | |

The labeling process is well adapted for high-throughput synthesis. This involves performing the photocoupling step in parallel. High-throughput-synthesis was demonstrated using four micro-vials containing PHPA-dye-entrapped nanoparticles and four different carbohydrates were coupled to the particles by a simultaneous photoactivated reaction (FIG. 1). The products showed successful conjugation of each carbohydrate to the dye-entrapped nanoparticles. Based on this result, the number of reaction wells can be further increased by using microarray technology to enable rapid parallel synthesis of larger libraries of dye-entrapped nanoparticles-labeled materials, such as biological molecules useful for specific binding pair reactions.

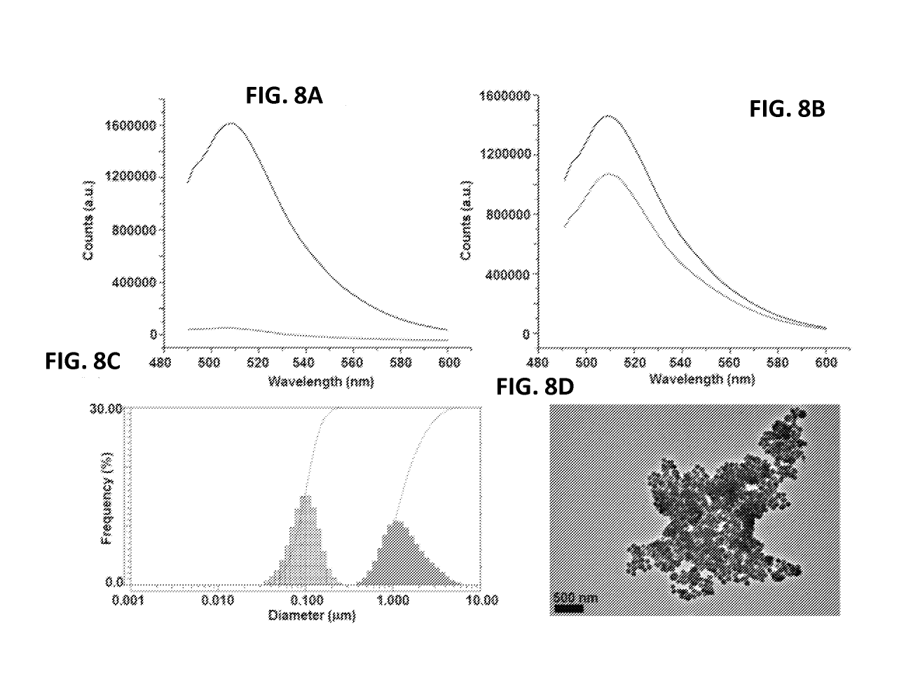

The bioactivity of carbohydrates labeled with dye-entrapped nanoparticles was then tested. D-Mannose labeled with fluorescein nanoparticles, FSNP-Man, was treated with Concanavalin A (Con A). Con A is a well-studied lectin that exhibits specific affinity to α-D-mannopyranoside, α-D-glucopyranoside, and their derivatives. At pH>7, Con A exists as a tetramer, inducing significant nanoparticle agglomeration upon binding to Man-containing ligands. When FSNP-Man was incubated with Con A for 1 hour, the fluorescence intensity of the solution decreased drastically (FIG. 8a). Precipitates were observed where the nanoparticles agglomerated into clusters (FIG. 8d). In contrast, when FSNP-labeled D-galactose, FSNP-Gal, was treated with Con A following the same procedure, the fluorescence intensity decreased only slightly (FIG. 8b). The small intensity decrease is likely due to the nonspecific adsorption of Con A on the nanoparticles. The FSNP-Man-Con A aggregates were additionally examined by dynamic light scattering (DLS), which showed, on the average, a 10-fold increase in the particle size in comparison to FSNP-Man (FIG. 8c).

Biological molecules labeled with nanoparticles comprising entrapped signal generating moieties, such as fluorescent dyes, have various utilities. For example, such materials can be used to detect biological molecules, to separate biological molecules, and to study the interactions of biological molecules. For example, oligosaccharides can be used to target various epitopes, such as for detection of cellular proliferative diseases, such as cancer. Table 3 below provides examples of certain antigens that can be targeted using the methods disclosed herein. A person of ordinary skill in the art will appreciate that the present invention is not limited to these particular targets, and rather that such list solely serves to exemplify targets in a sample.

TABLE 3
Examples of targeted di- and oligosaccharide structures

| Name | Structure | Name | Structure |
|---|---|---|---|
| lactose | Gal~1-4G1c | TF-antigen | Gal~1-3GaINAc |
| cellobiose | Glc~1-4G1c | H-antigen | Fuca1-2Gal-R |
| kojibiose | Glca1-2Glc | Tn-antigen | GalNAcaSer/Thr |
| chitobiose | GlcNAc~1-4GlcNAc | linear B | Gala1-3Gal-R |
| Man2a | Mana1-2Man | Lewis” | Gal~1-3(Fucal- |
| | | | 4)GlcNAc-R |
| Man3a | Mana1-2Mana1-2Man | Lewis” | Gal~1-4(Fucal- |
| | | | 3)GlcNAc-R |
| B-antigen | Gala1-3(Fuca1- | Forssman | GaINAca1- |
| | 2)Gal~1-3GlcNAc~ | | 3GaINAc~1-R |
| GM3 | Neu5Aca2-3Gal~1- | | 3Gala1- |
| | 4Glc~ | | 4Gal~1-4G1c-R |

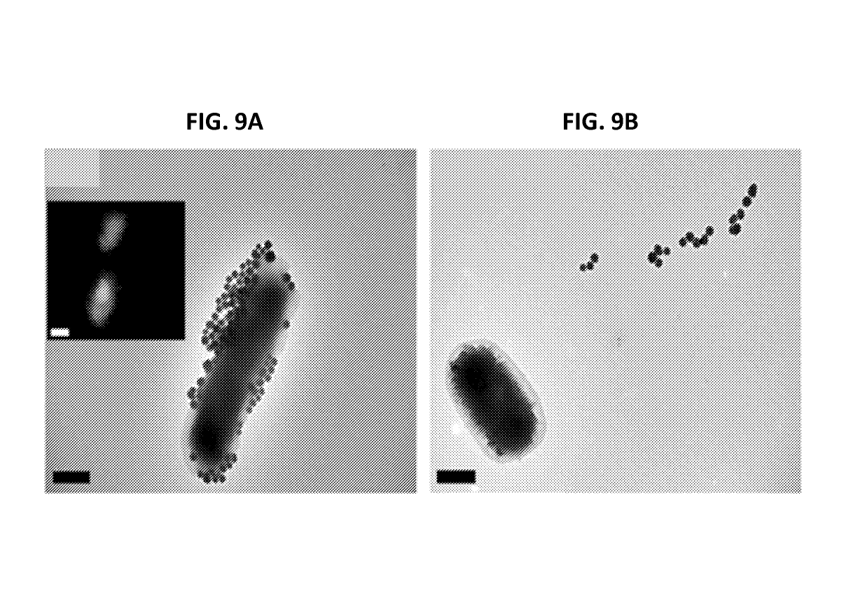

One working embodiment that establishes the utility of the nanoparticle labeling technique for labeling biological molecules concerned using Glyco FSNPs to image glycan-lectin interactions on a lectin microarray. For one embodiment, FSNP-Man was treated with E. coli bacteria strain ORN 178. E. coli strain ORN 178 contains a Man-specific binding domain, i.e., the FimH lectin, on type 1 pili. The TEM image of the resulting solution displays a large number of FSNP-Man on the E. coli (FIG. 9a). This results from the multivalent interactions between Man ligands on the FSNP-Man with the FimH lectin on the bacteria. The strong interaction was further confirmed by fluorescence imaging where intense fluorescence was observed on the bacteria (FIG. 9a insert). In contrast, when FSNP-Man was treated with E. coli strain ORN208 that is deficient of the Man-binding FimH protein, no fluorescence was observed on the bacteria. Instead, virtually no nanoparticles were attached to the bacteria surface (FIG. 9b).

The FSNP-labeled carbohydrates were next employed to study carbohydrate-lectin interactions on a lectin microarray. The lectin microarray was fabricated on aldehyde-functionalized glass slides following an established literature procedure.

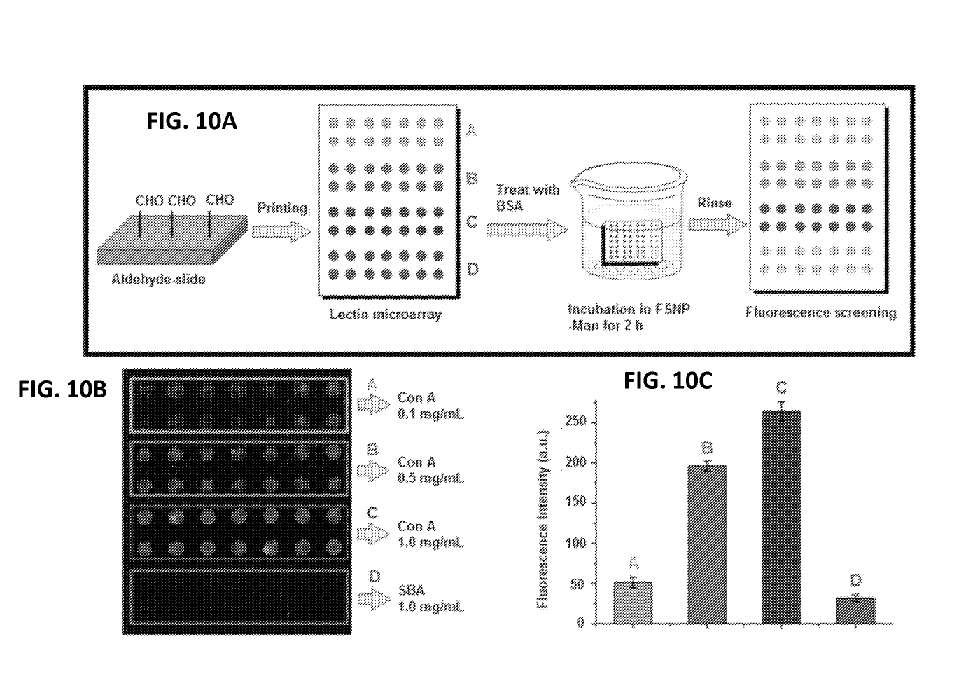

In summary, solutions of Con A and soybean agglutinin (SBA, a non-Man-binding lectin) in pH 7.4 phosphate-buffered saline (PBS) containing 40% glycerol were printed on aldehyde slides using a robotic printer. The lectin array slide, after blocking with bovine serum albumin (BSA), was incubated in FSNP-Man solution for 2 hours (FIG. 10a). As anticipated, fluorescence was observed on all Con A spots (FIG. 10b). The relative fluorescence intensities on Con A were approximately 10 times higher than those on SBA at the printing concentration of 1 mg/mL (FIG. 10c). Even at 0.1 mg/mL, the fluorescence intensity from Con A was still noticeably higher than that from SBA, although the spot quality deteriorated at lower printing concentrations. These results clearly demonstrate that the signal generating moiety-nanoparticle-labeled carbohydrates are highly suited for interrogating carbohydrate-lectin interactions using a microarray.

The above describes a general method for labeling carbohydrates with dye-doped silica nanoparticles. The strategy applies to underivatized carbohydrate structures, thus avoiding complex synthesis and purification steps that are often involved in the chemical derivatization of these ligands. The labeling is highly efficient, and the resulting FSNP-labeled carbohydrates retained their binding affinity and selectivity towards lectins. The utility of this labeling technique has been successfully demonstrated using FSNP-labeled carbohydrates to detect and image bacteria, and in probing glycan-lectin interactions on microarrays. These results illustrate that, although the labeling chemistry, i.e., the C—H insertion reaction of PHPA, yields a random orientation of the attached ligands, the labeled ligands retain their binding selectivities nonetheless. Further, the labeling reaction may allow the exposure of all epitopes on the ligands, and thus a biased epitope selection can be avoided.

The technique developed is readily applied to other biologically significant molecules including pharmaceuticals and metabolites. The advantage of this method resides in its generality and simplicity, where the labeling process employs a single labeling agent, PHPA-nanoparticle-signal generating moiety, and a uniform coupling chemistry. Ligands are labeled in their native forms without undergoing prior chemical derivatization. These features, combined with the straightforward preparation of signal generating moiety-doped nanoparticles, provide a myriad of uses for this technique in bioanalysis and diagnostic applications.

### VII. Blocking and Non-Specific Interactions with Substrate Surfaces, Such as Microarray Surfaces

Unlike organic dyes that are smaller than proteins and cause minimal changes in the non-specific interactions of the protein with the substrate, nanoparticles are large entities, and often can be equal in size or even larger than the proteins to be labeled. The property of nanoparticles thus becomes relevant and can impact the outcome of the interactions between NP-labeled biological molecules with the substrate.

An important goal of reporting the outcome of molecular recognition in sensing or imaging is a high signal/noise ratio. This goal is facilitated by increasing signals resulting from specific interactions and decreasing background noises due to non-specific adsorption. Polymer-based PHPA surfaces greatly enhance specific interaction signals. Using polymer-PFPA surfaces, high-density ligands and an antifouling coating can be immobilized simultaneously, leading to high specific signal and low background noise. This significantly enhances sensitivity of signals from, for example, microarrays. Disclosed embodiments of the present invention accomplish this goal by simultaneously increasing signal sensitivity (i.e., enhancing specific interactions) and reducing background noise (i.e. lowering non-specific adsorption) on a single platform. This is achieved using a highly efficient and uniform surface chemistry to immobilize both high-density ligands and the anti-fouling coating. This can be achieved in a single step by a fast and efficient light activation. The method eliminates the need for multiple or complex surface functionalization and synthesis. Furthermore, ligands and an anti-fouling coating can be applied at designated locations in a spatially-controlled fashion, and the process is readily integrated with existing microfabrication and array techniques.

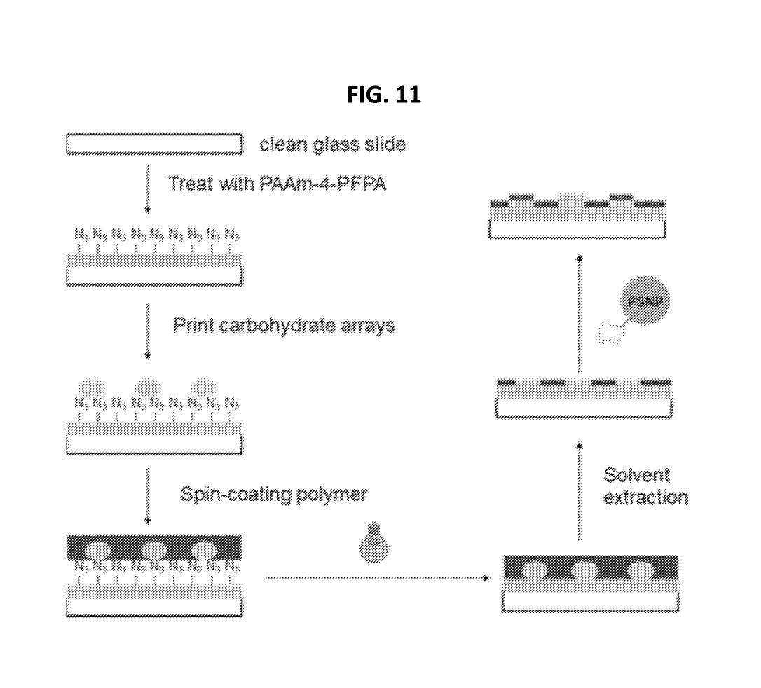

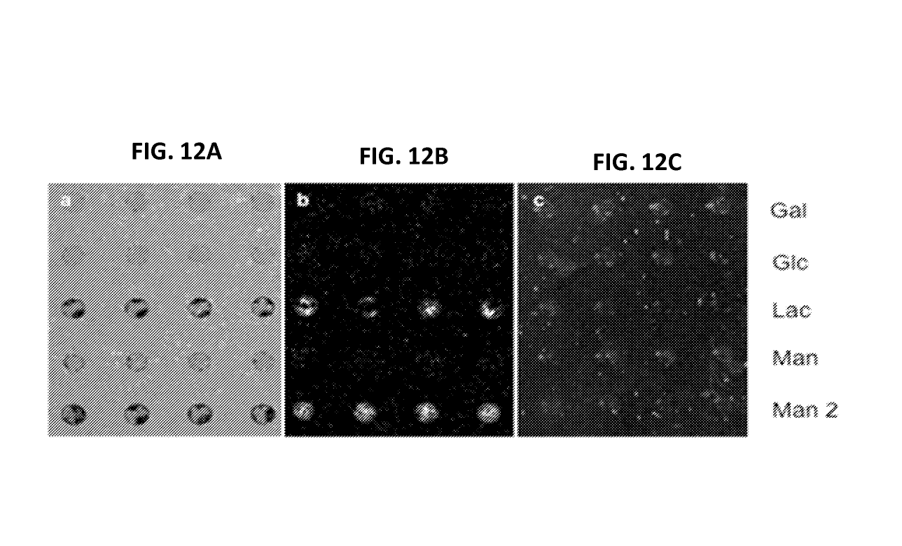

In order to investigate these issues, Con A was labeled with FITC-doped silica nanoparticles (FSNPs) to facilitate studying their interactions with carbohydrates. A carbohydrate array was generated according to the procedure in FIG. 11. In summary, carbohydrate ligands were printed on the PAAm-PFPA surface followed by spin-coating PEOX from a chloroform solution. This was done so as not to disturb carbohydrates printed on the surface. UV irradiation attached both carbohydrate ligands and PEOX on the surface. After solvent washing, a carbohydrate array was produced where the background was covered with PEOX. The array was then treated with FSNP-Con A. However, negative images were obtained where the carbohydrate ligands showed lower fluorescence intensity than the PEOX background (FIG. 12a).

Carbohydrate arrays were then made with PS instead of PEOX as the background coating. In this case, the fluorescence intensity was higher on the carbohydrate ligands than the PS background (FIG. 12b). BSA, a blocking agent that is frequently used in preventing non-specific absorption, also improved the background. But, the measured signal intensities were about 10 times lower than those when PS was used as the background (FIG. 12c).

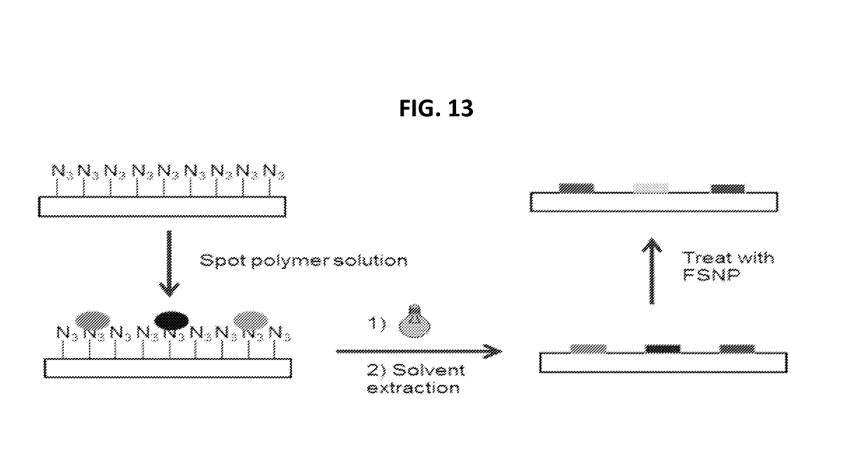

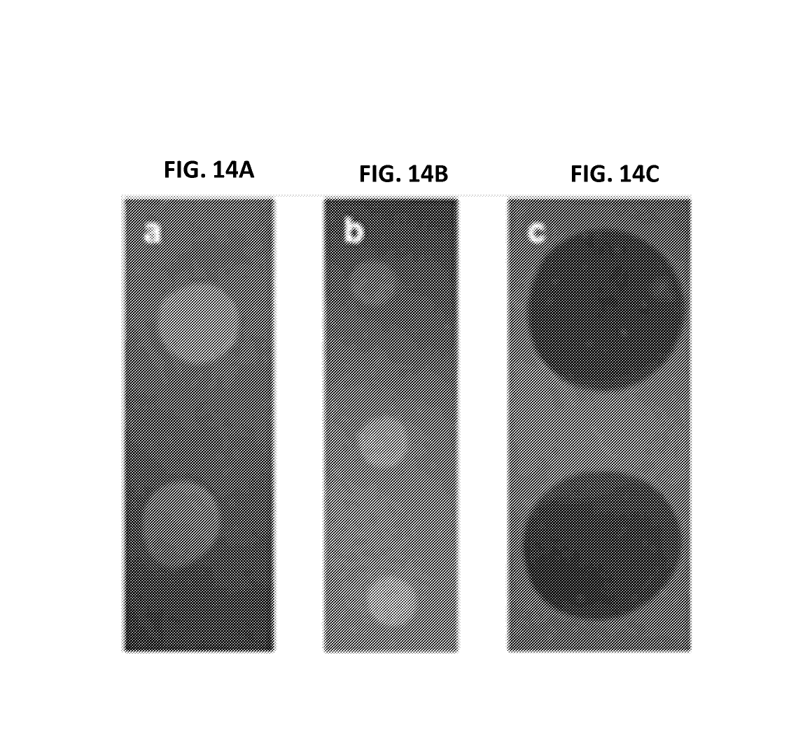

These results indicate that that the interaction of nanoparticles with surfaces may take a fundamentally different mechanism than that of proteins. While proteins are the least absorbed on hydrophilic surface such as PEO and PEOX, the silica nanoparticles have higher affinity for these surfaces. To further validate this result, solutions of PEOX, PEO, and PS were spotted on PFPA-functionalized slides using a pipet or tip, and the polymers were immobilized by UV activation. The sample was then treated with FSNPs. This process is illustrated schematically in FIG. 13. Bright spots were observed on PEOX and PEO (FIG. 14). For PS, however, the spots were significantly darker indicating that PS effectively prevented the non-specific adsorption of FSNPs (FIG. 14).

A variety of polymers can be selected to assess their interactions with nanoparticles, including charged, hydrophilic and hydrophobic polymers, with particular examples including the following polymers, where the numbers in parenthesis are the contact angle of the corresponding polymer: Poly(acrylic acid), Poly(vinyl alcohol) (51°), Poly(vinyl chloride) (86°), Poly(vinyl acetate) (61°), Polystyrene (88°), Poly(allyl amine), Poly(methyl methacrylate) (71°), Poly(n-butyl methacrylate) (91°), Poly(vinyl pyridine), Poly(ethylene terephthalate) (73°), Polybutadiene (96°), PEO/PEG, Polyoxymethylene (77°), Polypropylene (102°), PEOX, Poly(vinylidene chloride) (80°), Poly(t-butyl methacrylate) (108°) Poly(vinyl pyrolidone), Polycarbonate (82°), Polytetrafluoroethylene (109°). Dye-doped silica nanoparticles will be used in these studies as they provide a convenient way to visualize the interactions with the polymer arrays by fluorescence imaging.

### VIII. Polymer-PHPA Surfaces for Immobilizing Desired Molecules

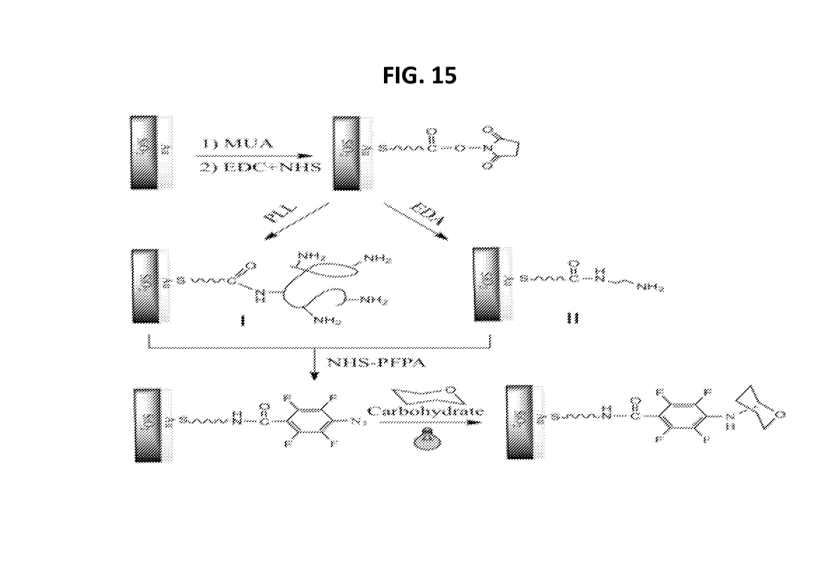

PHPA-polymer surfaces are more efficient than a PHPA surface alone for immobilizing desired molecules. This result has been established using poly(L-lysine) (PLL). A PLL-PFPA surface was prepared on gold films following the procedure in FIG. 15. The Au film was first functionalized with 11-mercaptoundecanoic acid (MUA), which was subsequently activated with N-hydroxysuccinimide (NHS) followed by treating the surface with PLL (I, FIG. 15). The density of amino groups on the PLL surface was determined as 37.3/nm² by colorimetry using 4-nitrobenzaldehyde. This is several folds higher than the surface that was treated with ethylenediamine (EDA) (II, FIG. 15), which gave an amine density of 8.1/nm².

The two surfaces were then treated with PFPA-NHS, and the resulting PFPA and PLL-PFPA surfaces were used to immobilize D-mannose (Man). The density of immobilized Man, 27.2/nm², was obtained on the PLL-PFPA surface determined by the anthrone-sulfuric acid method. On the PFPA surface, however, the amount of immobilized Man was below the detection limit of the assay. These results clearly demonstrated that higher ligand density is achieved on the PLL-PFPA than the PFPA surface. In fact, a wide variety of carbohydrate ligands from mono-, oligo- and poly-saccharides, as well as aldoses, ketoses, and even alditols, can be efficiently immobilized on the PLL-PFPA surface. Examples of such compounds include D-mannitol, D-fructose, D-trehalose, D-galactose, lactose, D-raffinose, dulcitol, D-mannosamine, L-arbinose, D-glucose, D-cellobiose, maltotetraose, D-sorbitol, D-glucosaminic acid, D-sorbose, D-mannose, D-sucrose, maltoheptaose, β-cyclodextrin, 10 kD dextran, 60-90 kD dextran.

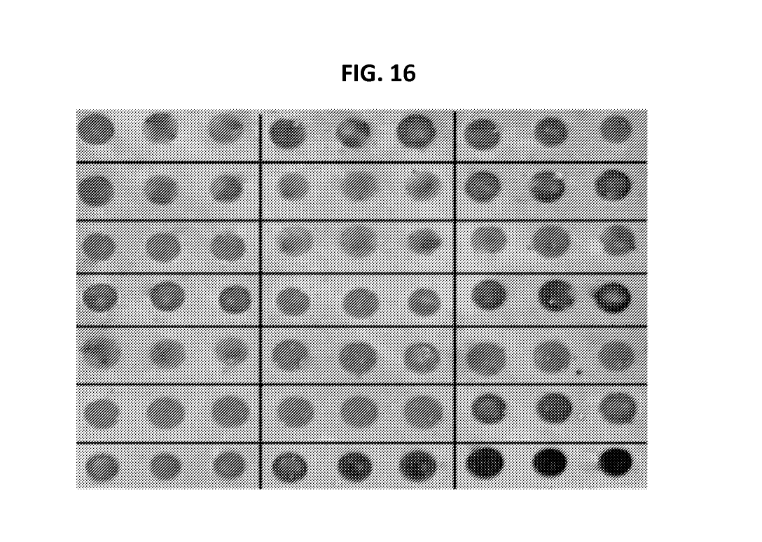

With reference to FIG. 16, however the spots were not uniform and showed the ring effect likely due to the hydrophobic PFPA surface. Ethanol amine was used, which is hydrophilic, to increase the affinity for carbohydrates. Indeed, the ring affect was mostly diminished with the addition of EOA to ethylene diamine (EDA). See, A Universal Protocol for Photochemical Covalent Immobilization off Intact Carbohydrates for the Preparation of Carbohydrate Microarrays, Wang et al., Bioconjugate Chem., 2011, 22, 26-32, which is incorporated herein by reference.

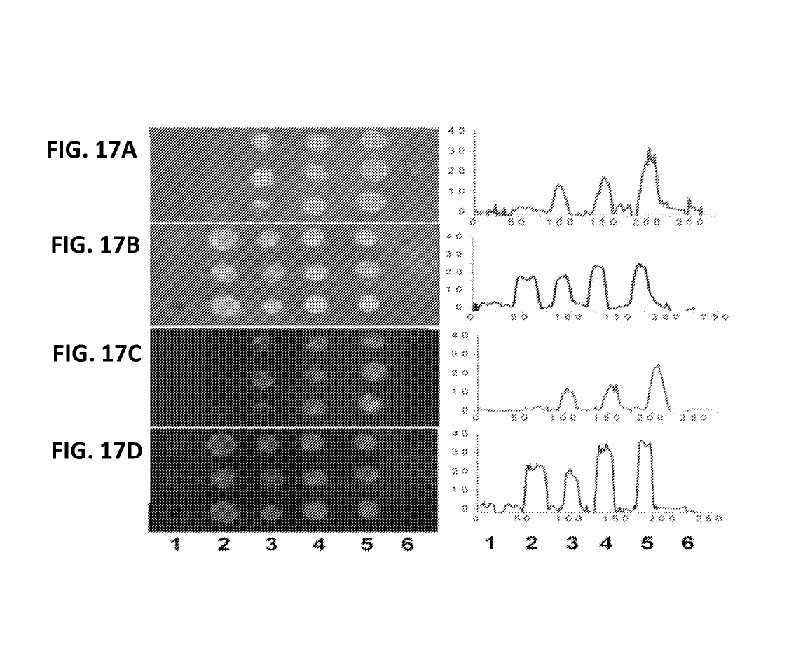

In addition, the PLL-PFPA surface significantly enhanced the interactions between immobilized ligands and proteins. FIG. 17 shows the surface plasmon resonance (SPR) and fluorescence images of the immobilized carbohydrates after they were treated with Concanavalin A (Con A), a lectin that binds carbohydrates having α-Man and α-glucose (Glc) structures. The signals were uniform and of high intensities on samples prepared on the PLL-PFPA surface (b, d, FIG. 17). On the PFPA surface, however, signals were weaker and of lower intensity especially for mono- and oligo-saccharides (1 and 2 in a and c, FIG. 17). This clearly demonstrated the advantages of a polymer-based PFPA surface in increasing ligand density and enhancing the ligand interactions with protein.

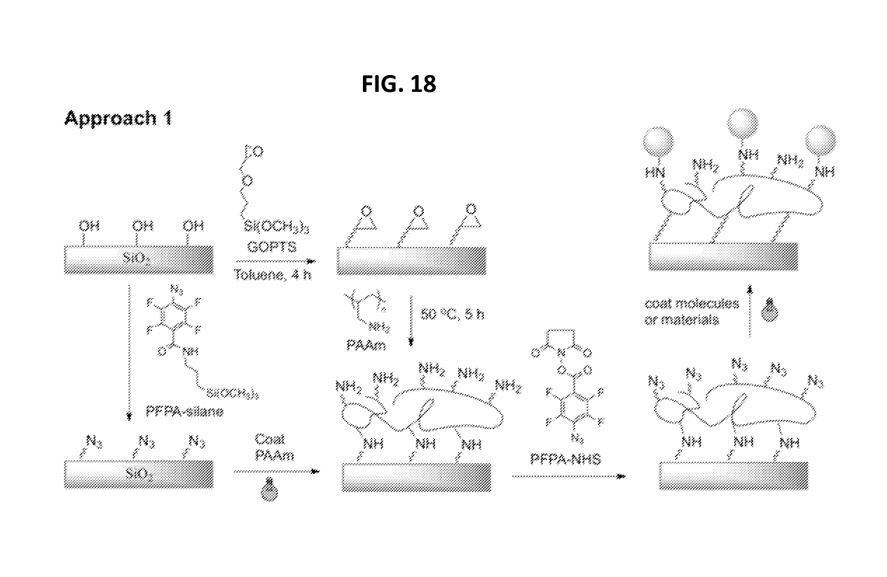

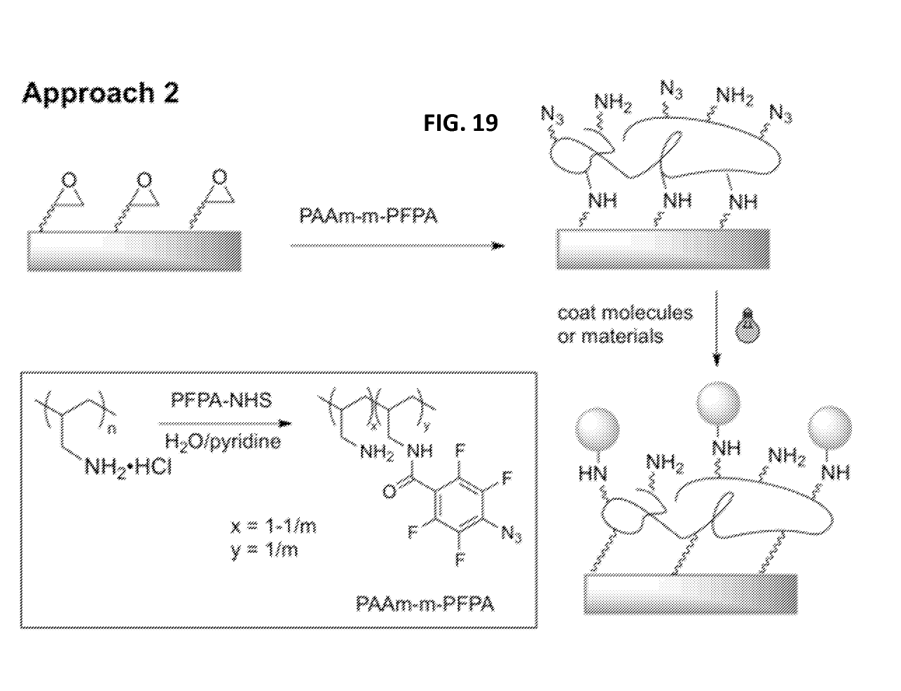

Poly(allylamine) (PAAm) also can be used as a polymer matrix. Like PLL, PAAm is a polyamine, however, it is at least 10 times less expensive than PLL. Two approaches can be used to make PAAm-based PFPA surfaces (FIGS. 16-17). In the first approach, PAAm-PFPA will be prepared by immobilizing PAAm on the surface followed by PFPA-NHS (FIG. 18). Glass slides will be treated with 3-glycidyloxytrimethoxysilane (GOPTS) by soaking Piranha-cleaned glass slides in a 12 mM solution of GOPTS in toluene for 4 hours followed by curing at room temperature overnight. The expoxy slides will then be incubated in the PAAm solution at 50° C. for 5 hours to covalently attach the polymer to the surface (FIG. 18). Alternatively, PAAm can be covalently immobilized on PFPA-functionalized glass slides using our photocoupling chemistry (FIG. 19). In this case, glass slides will be treated with PFPA-silane. PAAm will then be coated and covalently immobilized by irradiating the sample with a medium pressure Hg lamp for 5 minutes followed by removing the excess PAAm with extensive rinsing.

The PLL-based matrix was the most effective for mono- and oligo-saccharides where high surface PFPA density is needed. For large molecules such as dextran and synthetic polymers, the surface PFPA density is less critical. Since only one attachment point is necessary for the entire polymer to be immobilized, these macromolecules can tolerate significantly lower PFPA density. Even with a few percent of surface PFPA coverage, uniform polymer films could be obtained.

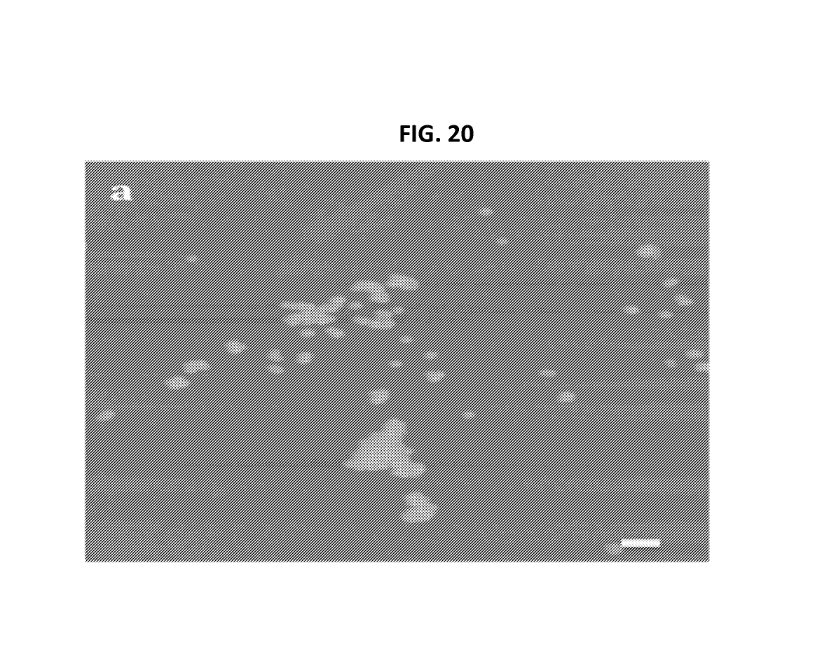

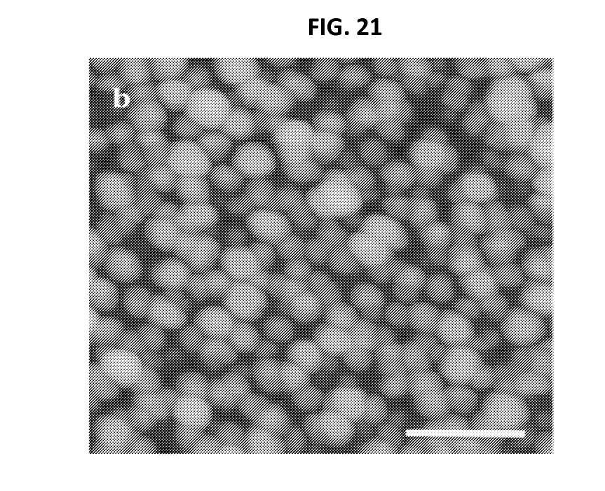

Nanoparticles, such as silica nanoparticles, also have been immobilized on PHPA surfaces. Nanoparticles do attach, but at a relatively low density, as determined by AFM (FIG. 20). In order to form the covalent linkage, the material needs to be in close proximity to the PFPA surface. The “hard” silica nanoparticles may not form close contact with the equally “hard” PFPA surface. The spherical geometry of the nanoparticles also reduces the contact area with the PFPA surface. The PAAm-PFPA matrix, however, is softer and can additionally reduce steric hindrance which may enhance the contact between the PAAm-PFPA surface and the nanoparticles. In fact, when the PAAm-PFPA surface was used, the same nanoparticles were successfully immobilized with high particle density (FIG. 21).

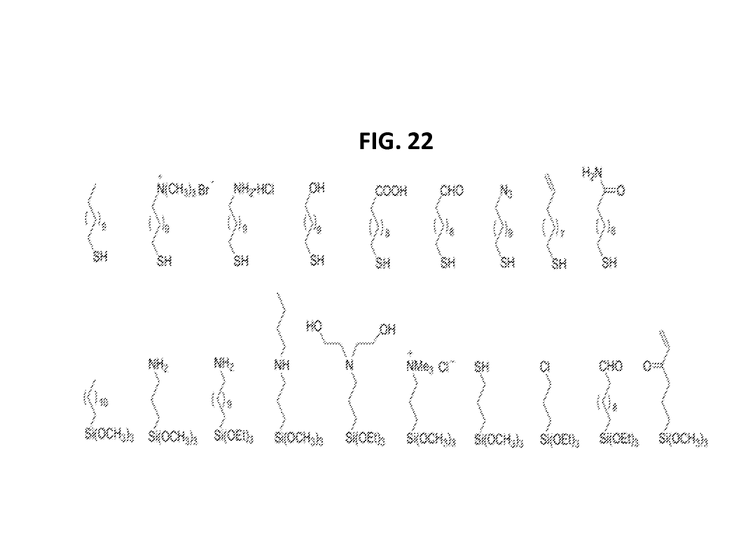

Nanoparticles of different types, sizes, and surface functionalities will be immobilized on a polymer-PHPA surface. Gold nanoparticles will be synthesized following established Turkevich or Brust procedures. Particle sizes ranging from a few nm to several tens of nm can be readily prepared using these methods. Silica nanoparticles will be prepared by the Stöber procedure. Monodisperse particles from tens of nm up to μm can be synthesized. Also important is the surface functionality. These particles will be functionalized with polar, non-polar, or charged groups to study the impact of surface property on the particle immobilization efficiency. Various examples of commercially available thiol and silane structures useful for functionalization are provided in FIG. 22.

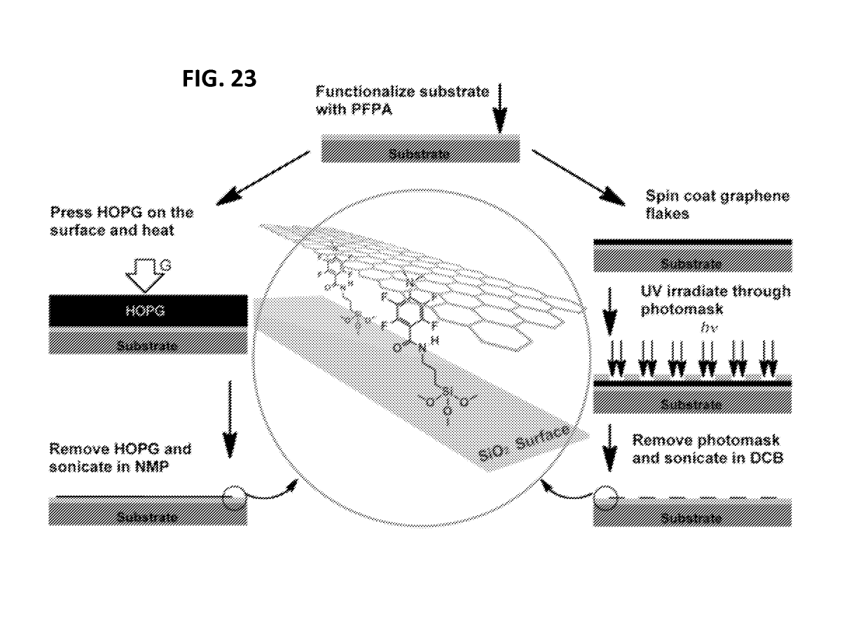

Graphene also can be immobilized on a surface. See, for example, U.S. patent application Ser. No. 12/455,269, which is incorporated herein by reference. This has been accomplished by pressing and heating highly oriented pyrolytic graphite (HOPG) on the PHPA surface (FIG. 23a) or by spin coating graphene flakes on PFPA surface followed by UV irradiation (FIG. 23b). These results show the power of PFPA chemistry in functionalizing graphene, however, the graphene films were sparsely attached on samples prepared by pressing HOPG on the PFPA surface.

PAAm-PFPA surface should markedly improve the immobilization efficiency of graphene. The thicker and softer PAAm matrix will likely enhance the contact of HOPG and graphene flakes making them more accessible for the covalent bond formation. These embodiments will be carried out following the same procedures shown in FIG. 23a and FIG. 23b except that the substrate, i.e., silicon wafer, will be functionalized with PAAm-PFPA.

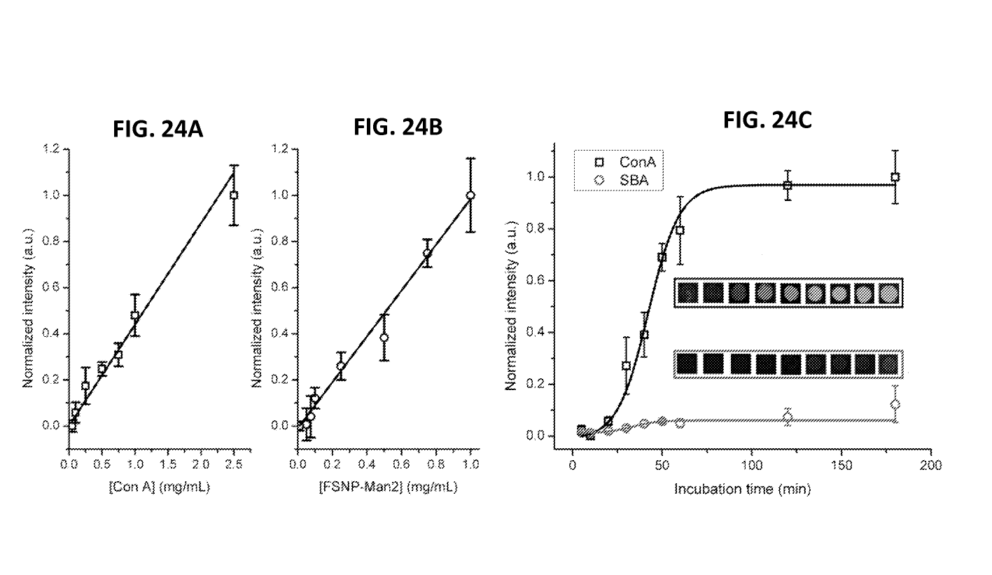

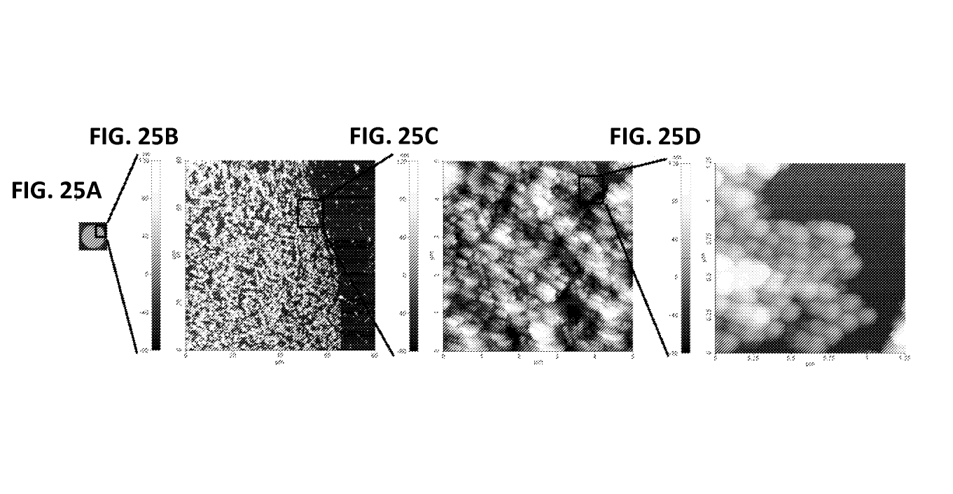

Particular disclosed embodiments concern using the disclosed conjugate to perform a plurality of microarrays using plural different targets. In particular disclosed embodiments, the lectin printing concentration, the glyco-FSNPs concentration, and the incubation time for various microarrays were varied to study the impact of these varying conditions on the binding results. FIGS. 24(a-c) illustrate the results obtained during optimization of this particular microarray. FIG. 24a illustrates a particular working embodiment that indicates when Man2-FSNPs were incubated with Con A, the fluorescence intensity increased linearly with the Con A printing concentration, which ranged from 0.1 to 2.5 mg/mL. Similarly, the fluorescence intensity increased with the concentrations of Man2-FSNPs, which ranged from 0.05 to 1.0 mg/mL (FIG. 24b). The incubation time was then varied while keeping concentrations of Con A and Man2-FSNPs at 1 mg/mL, and 1.5 mg/mL, respectively. As shown in FIG. 24c, the intensity increased until 75 min and reached saturation after 2 hours. The incubated lectin spots were characterized by AFM. Images show that the spot was fairly uniformly covered with FSNPs [FIG. 25(a-d)]. More than a single layer of FSNPs was observed in some areas, which, without being limited to a particular theory of operation, is currently believed to be due to nanoparticle agglomeration before and/or during incubation. A non-binding lectin, SBA, was used as a control and was treated with Man2-FSNPs. In this embodiment, the signals remained low until after 2 hours, and increased afterwards because of the non-specific adsorption (FIG. 24c). To minimize particle agglomeration and non-specific adsorption, the glyco-FSNP concentration and the incubation time were kept at 1.5 mg/mL and 2 hours, respectively, for subsequent studies.

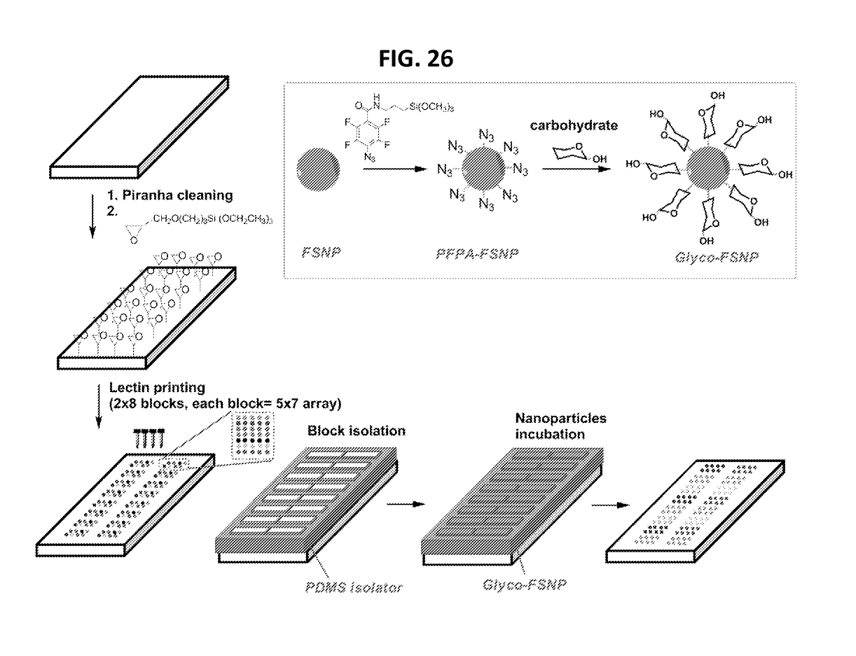

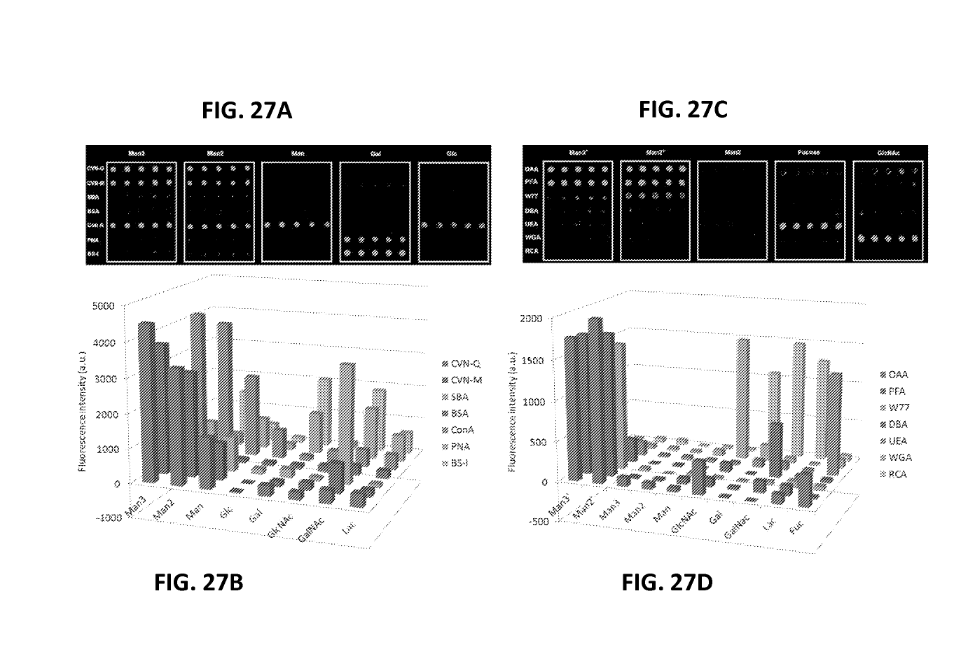

In a particular working embodiment, a total of 14 lectins were used in a microarray; their binding carbohydrate ligands are listed in Table 4. This super-microarray illustrates the ability of the disclosed conjugate and method to perform in detecting multiple different targets. Due to the size limitation of the PDMD isolator and printing spot size, only 7 lectins could fit in one isolator well, and therefore, the 14 lectins were divided into two groups. The lectins were printed on GPTMS-functionalized glass slides in a 7×5 array, and the array was repeated in 2×8 array format to create 16 microarrays on each chip (FIG. 26). The particular carbohydrate ligands used include: Man, Glc, Gal, GlcNAc, GalNAc, Fuc, 2α-Man2, 6α-Man2, 2α,2α-Man3, 3α,6α-Man3. Each ligand was coupled on FSNPs as previously disclosed. A 2×8 PDMS isolator was placed on the lectin super-microarray slide, and different glyco-FSNPs were incubated in each individual lectin array.

TABLE 4
Lectins and their corresponding carbohydrate binding ligands.

| Lectin | | Origin | Binding ligands |
|---|---|---|---|
| CVN-Q | Cyanovirin-N Q^{50C} | Cyanobacteria | α-1,2-Man |
| CVN-M | Cyanovirin-N Mut^{DB} | Cyanobacteria | α-1,2-Man |
| SBA | Soybean Agglutinin | Glycine max | GalNAc |
| | | (soybean) | |
| Con A | Concanavalin A | Canavalia | Man |
| | | ensiformis | |
| PNA | Peanut Agglutinin | Arachis hypogaea | Gal(β- |
| | | (peanut) | 1,3)GalNAc/Gal |
| BS-I | Bandeiraea | Griffonia | GalNAc, and Gal |
| | simplicifolia | (Bandeiraea) | |
| | | simplicifolia seeds | |
| OAA | Oscillatoria Agardhii | Cyanobacteria | α-1,6-Man |
| | Agglutinin | | |
| PFA | Homolog of OAA | N/A | α-1,6-Man |
| W77 | Mutant of OAA | Cyanobacteria | α-1,6-Man |
| DBA | Dolichos Biflorus | Dolichos biflorus | GalNAc |
| | Agglutinin | (horse gram) seeds | |
| UEA | Ulex Europaeus | Ulex europaeus | L-fucose |
| | Agglutinin I | (Furze gorse) seed | |
| WGA | Wheat Germ | Triticum vulgaris | GlcNAc |
| | Agglutinin | (wheat germ) | |
| RCA | Ricinus Communis | Ricinus communis | Gal/GalNAc/Lac |
| | Agglutinin I | (castor bean) seeds | |

Results of the microarray analysis are shown in FIG. 26(a-d). The binding pairs such as 2α-Man2/2α, 2α-Man3 with CVNs/Con A, Man with Con A, Gal with PNA/BS-I, as well as 6α-Man2/3α, 6α-Man3 with OAA/PFA, Fuc with UEA and Glc with WGA show noticeably higher intensities than those non-binding pairs (association constants as shown in Table 5), demonstrating high selectivity of the super-microarrays. The signal intensities also revealed the relative binding affinities of glycan ligands with lectins. For instance, Con A that is a tetrameric protein having saccharides binding sites to α-Man and α-Glc in the affinity order of 2α,2α-Man3 (3.79×10⁵ M⁻¹)>2α-Man2 (4.17×10⁴ M⁻¹)>Man (8.2×10³ M⁻¹)>Glc (1.96×10³ M⁻¹) according to the reported solution association constants (Kₐ). In this study, Con A spots showed highest intensities for 2α,2α-Man3, and lowest for Glc and derivatives (GlcNAc and Lac), whereas almost no signal for non-binding ligand Gal. Another group of lectins of interest were CV-N mutants: Q^{50C} and Mut^{DB}, anti-HIV lectins that recognize α-1,2 linked Man2 and have been studied on gold nanoparticle platform. For example, in the previous work of Barrientos et al., the association constants of CV-N^{MutDB} were determined as 2.94×10⁵ M⁻¹ for 2α,2α-Man3 and 1.32×10³ M⁻¹ for 2α-Man2. In the microarray studies, 2α,2α-Man3 conjugated FSNP showed higher fluorescent density than Man2-FSNP, which correlated well with the affinity ranking in the ITC and gold GNP studies. Similar results were also observed for GalNAc attached FSNP with it binding lectin SBA (Kₐ=1.51×10⁶ M⁻¹). Also, absolute intensities were correlated to the association constant. For instance, intensities of CVN-Man3, Con A-Man3, SBA-GalNAc whose Kₐ were in range of 10⁵-10⁶ M⁻¹, were much higher those of Con A-Man, PNA-Gal, DBA-GalNAc and RCA-Gal (Kₐ in 10³ M⁻¹).

TABLE 5
Association constants (Kₐ) of lectins and glycans.

| Lectin | Glycan | Kₐ (M⁻¹) |
|---|---|---|
| CVN-Q²¹ | 2α-Man2 | 1.43 × 10³ (Domain A); |
| | | 1.56 × 10⁴ (Domain B) |
| | 2α, 2α-Man3 | 2.94 × 10⁵ (Domain A); |
| | | 2.33 × 10⁴ (Domain B) |
| CVN-M ²² | 2α-Man2 | 1.32 × 10³ |
| | 2α, 2α-Man3 | 2.94 × 10⁵ |
| Con A | Man | 8.2 × 10³ |
| | 2α-Man2 | 4.17 × 10⁴ |
| | 2α, 2α-Man3 | 3.79 × 10⁵ |
| SBA | Glc | 1.96 × 10³ |
| | GalNAc | 1.51 × 10⁶ |
| PNA | Gal | 0.98 × 10³ |
| | GalNAc | 2.43 × 10³ |
| BS-I | Gal | 2.1 × 10⁴ |
| | GalNAc | 1.87 × 10⁵ |
| DBA | GalNAc | 4.2 × 10³ |
| WGA | GlcNAc | 0.4 × 10³ |
| RCA | Gal | 2.2 × 10³ |
| | GalNAc | 4.84 × 10⁴ |

The 2×8 PDMS isolator creates 16 individual microarrays, and therefore, 16 different assays can be carried out simultaneously on a single super-microarray. When 16 ligand competition assays are performed, a dose-response curve can be plotted, from which the IC₅₀ value will be derived and the d constant subsequently calculated. This provides a high throughput means to determine binding affinity from a single super-microarray.

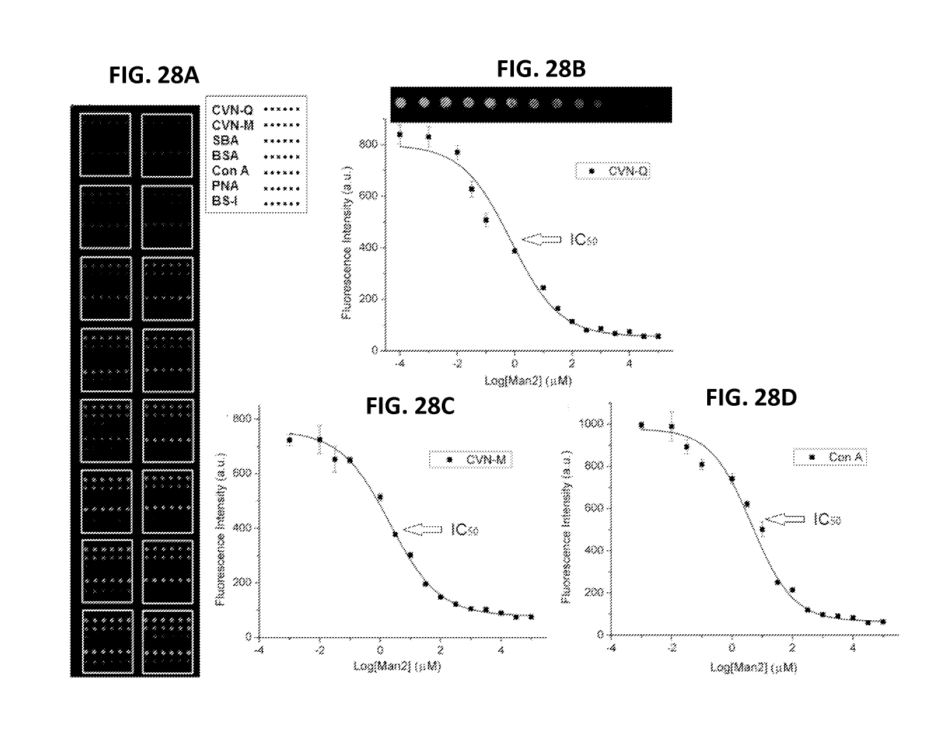

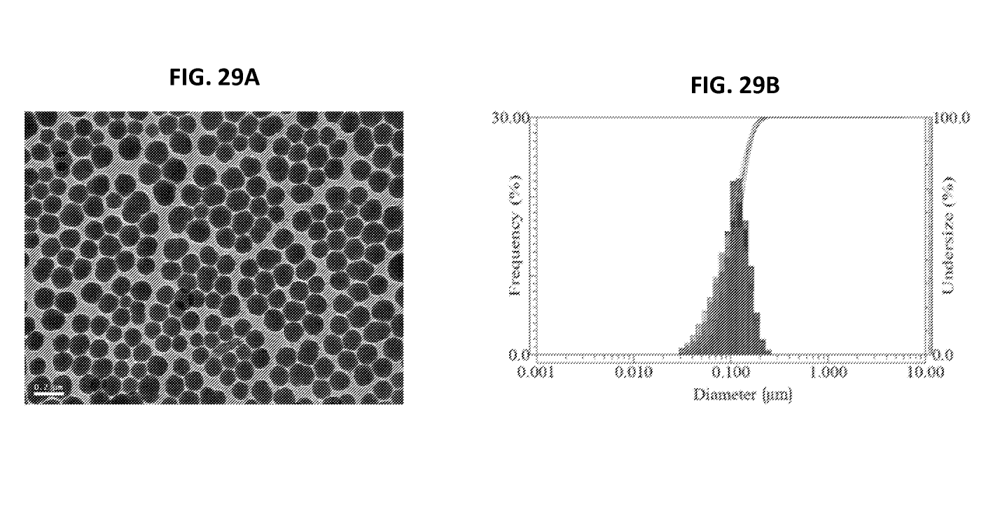

Competition binding assays were performed by mixing free competing ligands of varying concentrations with a fixed concentration of glycol-FSNPs and the resulting solutions were transferred to the wells on the super-microarray. FIG. 28a showed a typical fluorescence image of the lectin super-microarray after incubating with Man2-FSNP and varying concentration of 2α-Man2. It can be seen that only the binding lectins for 2α-Man2 (CVN-Q, CVN-M and Con A) exhibited strong and different signals with varying concentrations of 2α-Man2. Further, the fluorescence intensity decreased with increasing 2α-Man2 concentration, which was anticipated since 2α-Man2 competed with Man2-FSNP for binding the lectins on the array. By plotting the fluorescence intensity against the concentration of the free ligand, a dose response curve was obtained, from which the IC₅₀ value can be derived. FIG. 28b-d illustrate the results from the three binding lectins CVN-Q, CVN-M and Con A. Following a revised Cheng-Prusoff equation [K_{D}=IC₅₀/(1+[M]/K_{d}), where K_{d} is the dissociation constant of the free ligand with the corresponding lectin, K_{D}), the apparent dissociation constant of immobilized lectin with glycol-FSNP was calculated, and results are shown in Table 6.

TABLE 6
Apparent KD values of glyco-FSNPs with lectins obtained
from super-microarrays.

| | Ligand | | | | |
|---|---|---|---|---|---|
| | density | | | | |
| | (ligand/nm²) | CVN-Q | CVN-M | Con A | PNA |
| FSNP- | 1.3 | 1.66 nM | 0.43 nM | 0.0083 nM | N/D³ |
| Man3 | | (15.5 μM) | (3.4 μM) | (2.97 μM) | |
| FSNP- | 1.5 | 16.6 nM | 589 nM | 5.40 nM | N/D |
| Man2 | | (89.8 μM) | (757 μM) | (24 μM) | |
| FSNP- | 4.2 | N/D | N/D | 137 nM | N/D |
| Man | | | | (470 μM) | |
| FSNP- | 4.3 | N/D | N/D | N/D | 114 nM |
| Gal | | | | | (1050 μM) |

²The data in brackets are dissociation constants of the free ligand with the lectin.
³N/D = not detectable.

A dramatic increase in affinity was observed when glycans attached on nanoparticle surface. Results showed that the multivalent presentation of carbohydrate ligands significantly enhanced the binding affinity of FSNPs by 4-6 orders of magnitude in comparison to the free ligands in solution. In addition, the apparent affinity of FSNP-Gal with its binding lectin PNA was determined as well, and similarly K_{D} was 7 orders of magnitude lower than K_{d} of free galactose ligand. Those K_{D} values (Table 7) obtained were comparable to with using other microarray platform, such as surface plasmon resonance, and evanescent-field fluorescence.

TABLE 7
A summary of all KD values obtained using different methods.

| | | | | Kd | |
|---|---|---|---|---|---|
| Platform | | Method | Ligands | (nM) | Reference |
| Solution | | ITC | Man | 750 μM | Brewer's paper |
| Flat substrate | | QCM | Man | 540 | Pei. Y.; et al. |
| (2D) | | | | | Anal. Chem., |
| | | | | | 2007, 79, 6897. |
| | | SPR | Man | 180 | Smith, E. A.; et |
| | | | | | al.; J. Am. Chem. |
| | | | | | Soc. 2003, 125, |
| | | | | | 6140 |
| Nano- | 2D- | SPR | GNP- | 2.3-88 | Lin, C.-C.; et al. |
| particles | based | competition | Man | | Chem. Commun. |
| (3D) | | | | | 2003, 23, 2920 |
| | | QCM | GNP- | 63 | Mahon, E.; et al. |
| | | | Man | | Chem. Commun. |
| | | | | | 2010, 46, 5491 |
| | | Microarray | FSNP- | 14 | Present disclosure |
| | | competition | Man | | |
| | 3D- | UV-vis | GNP- | 13 | Chuang, Y. J; et |
| | based | | Man | | al.. Biochem. |
| | | | | | Biophys. Res. |
| | | | | | Commun. 2009, |
| | | | | | 389, 22. |
| | | Fluorescence | GNP- | 19 | Present disclosure |
| | | competition | Man | | |
| | | DLS | GNP- | 86 | Present disclosure |
| | | | Man | | |
| | | | SiNP- | 63 | Present disclosure |
| | | | Man | | |
| | | ITC | GNP- | 122 | Present disclosure |
| | | | Man | | |

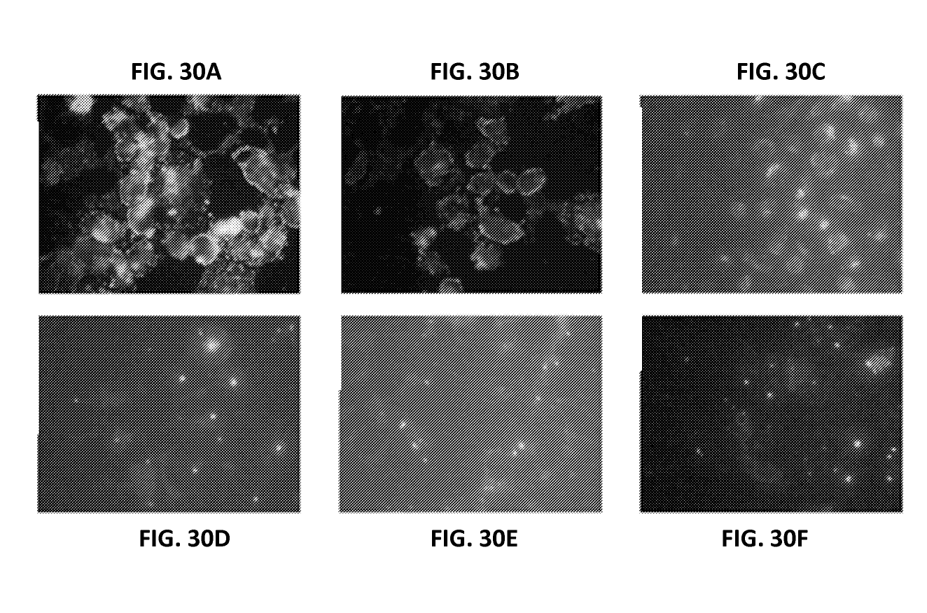

In particular disclosed embodiments, the disclosed conjugate may be used to bind to and ultimately detect prostate cancer cells, such as PC3. In exemplary working embodiments, the binding of carbohydrate-conjugated FSNPs with PC3 was examined by mixing embodiments of the disclosed conjugate with prostate cancer cells (PC3) which attached at the bottom of tissue culture plates. In certain embodiments, benign hyperplastic prostatic epithelial cell (BPH1) was used as a control cell line, and cellobiose was used as a control ligand that has the same chemical formula but different structure with that of lactose, a specific binding carbohydrate for Gal-1. Nanoparticle surfaces were blocked by Bovine serum albumin (BSA) to avoid non-specific bindings. After incubation, a portion of the supernatant was withdrew and measured by a spectrofluorometer to obtain the fluorescent intensity which was compared with that of the original solutions before incubation. Meanwhile, cells were washed with HEPES buffer (pH=7.5) and imaged by a fluorescent microscope. As shown in FIG. 30, when nanoparticle surfaces were not blocked by BSA, both lactose- and cellobiose-conjugated FSNPs could bind to PC3 cells (FIG. 30a,b), and lactose-conjugated nanoparticles also bind to BPH1 cells (FIG. 30c). However, when nanoparticle surfaces were blocked by BSA, all the bindings were significantly diminished (FIG. 30d-f). These observations suggest that the non-specific binding is dominant when the nanoparticle surfaces were not blocked by BSA, while the presence of BSA on nanoparticle surfaces can greatly hinder the specific binding as well. The role of BSA in blocking specific bindings was further confirmed by the results of fluorescent intensity measurements. BSA blocking on nanoparticle surfaces led to about 20% of nanoparticles binding to cells, while non-BSA blocking resulted in about 80% of nanoparticles binding to cells.

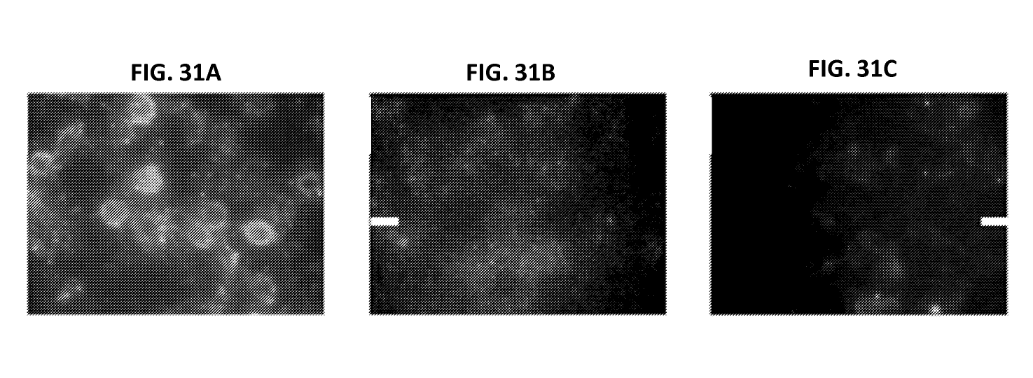

Given the fact that Gal-1 is actually expressed on PC3 surfaces, it was assumed that the binding affinity between lactose-conjugated FSNPs and Gal-1 on cell surfaces might not be strong enough to compete with BSA blocking. Consequently, the role of dithiothreitol (DTT) in improving interactions between carbohydrate-conjugated FSNPs and prostate cancer cells was investigated. As a widely-used reducing agent, DTT prevents both intra-molecular and inter-molecular disulfide bonds from forming between cysteine residues of Gal-1, thereby keeping Gal-1 in a more activated form. As shown in FIG. 31a, the binding of lactose-conjugated FSNPs (blocked with BSA) with PC3 cells is clearly visible after the cells were pretreated with DTT. Most of FSNPs were attaching to the cell surfaces, while internalizations of certain amount of nanoparticles into cells may occur as well. In control embodiments, no-ligand-conjugated FSNPs showed much lower extent of binding to PC3 cells (FIG. 31b), while lactose-conjugated FSNPs also showed greatly reduced binding to BPH1 (FIG. 31c). These results indicate that lactose-conjugated FSNPs could specifically bind to prostate cancer cells under appropriate conditions.

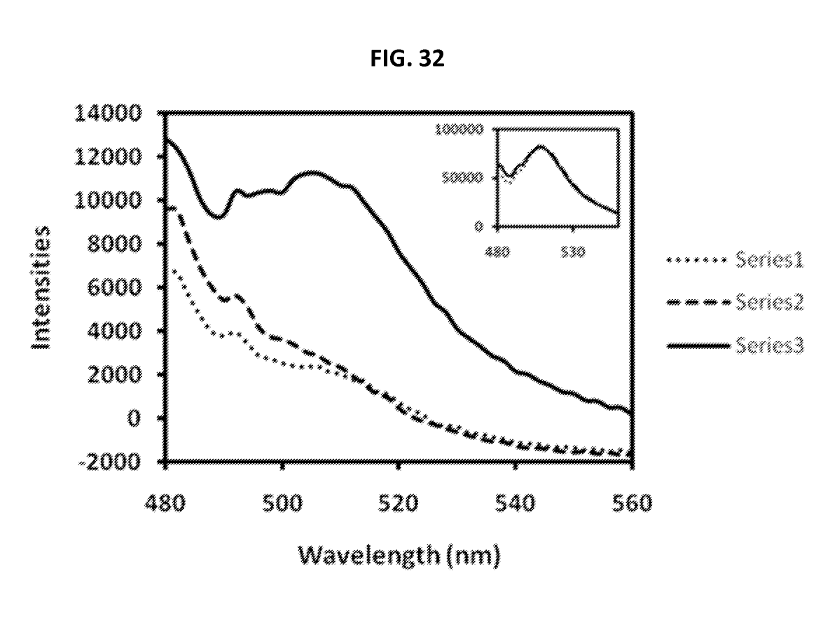

The specific binding of lactose-conjugated FSNPs with prostate cancer cells in presence of DTT was further confirmed by the measurement of fluorescent intensities of NP-bound cells by a spectrofluorometer. FIG. 32 shows that PC3 cells exhibited higher fluorescent intensities (emission at 510 nm) when bound with lactose-conjugated FSNPs, indicating that there were apparently more lactose-conjugated FSNPs binding to PC3 cells than cellobiose-conjugated or no-ligand-conjugated FSNPs.

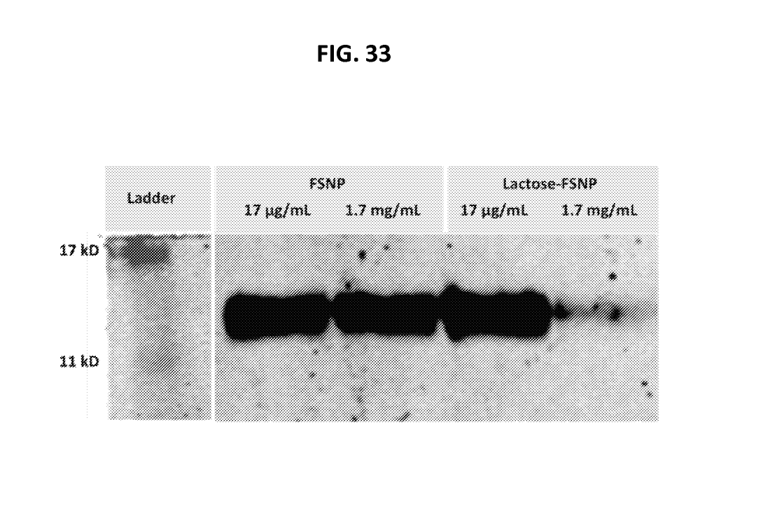

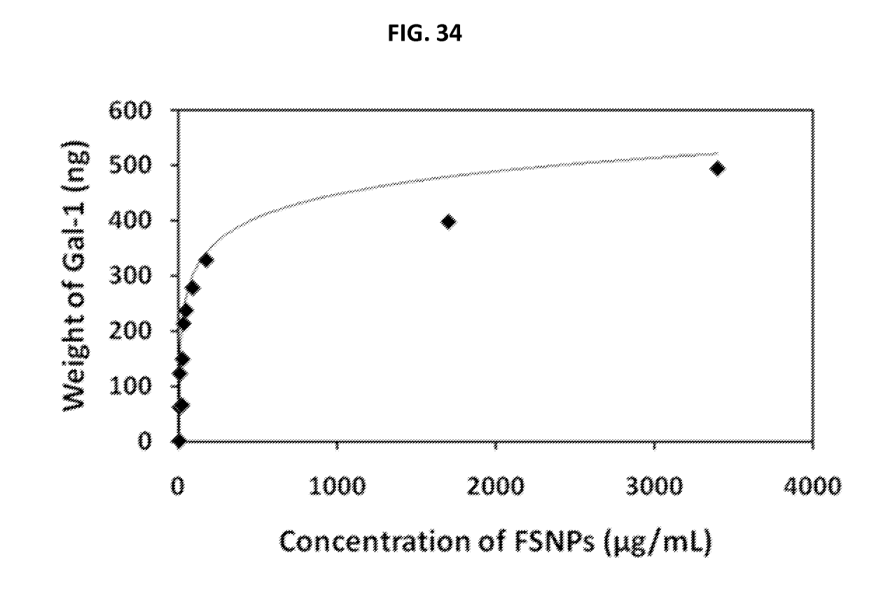

Another proof-of-concept of specific bindings between lactose-conjugated FSNPs and Gal-1 was established by the western-blotting, a widely-used electrophoretic method for studying expressions of specific proteins. Presumably, lactose-conjugated FSNPs could bind to Gal-1, leading to a decreased amount of Gal-1 remained in the solution after the removal of NPs by centrifugation. Therefore, the binding between nanoparticles and cells could be evaluated, both qualitatively and quantitatively, by analyzing the expression of Gal-1 remained in the solution. As shown in FIG. 33, when being mixed with no-ligand-conjugated FSNPs, the amount of Gal-1 did not change despite of varying concentrations of nanoparticles, suggesting that bare nanoparticles cannot bind to Gal-1. On the other hand, after being mixed with lactose-conjugated FSNPs at a higher concentration, the amount of Gal-1 significantly decreased as compared with that at lower nanoparticle concentrations. These results confirm the binding affinities of lactose-conjugated NPs toward Gal-1. The amount of Gal-1 bound to FSNPs as a function of nanoparticle concentrations was then evaluated by measuring the optical densities of the blots and the subsequent normalizations with control samples. The resulting weights of Gal-1 bound to FSNPs as a function of FSNP concentrations in FIG. 34 were subsequently fitted to the Langmuir adsorption isotherm given in equation 1:

$${W}_{Gal 1} = \frac{{W}_{Max} {K}_{L} {C}_{NP}}{1 + {K}_{L} {C}_{NP}} \qquad ( 1 )$$

in which W_{Gal1} is the weight of Gal-1 bound to FSNPs, Wₘₐₓ is the maximum weight of Gal-1 (500 ng), K_{L} is a binding constant, and CFSNP is the concentration of FSNPs. The fit of the Langmuir model gave the value of 2.766×10⁻² (μg/mL)⁻¹ for K_{L}. Hence the binding affinity (Kₐ, M⁻¹) of Gal-1 with lactose-conjugated FSNPs could be calculated as long as the density of lactose on FSNP surfaces is known.

### IX. Examples

### Materials and Instrumentation

3-aminopropyltriethoxysilane (APTMS), 3-glycidyloxytrimethoxy silane (GOPTS), D-(+)-mannose (Man), D-(+)-glucose (Glc), D-(+)-galactose (Gal), maltopentaose (Glc5) were obtained from TCI America. Anthrone (97%), FITC (Fluorescein isothiocyanate isomer I, 90%), tetraethyl orthosilicate (TEOS), Con A (lectin from Canavalia ensiformis (Jack bean), Type IV), SBA (from Glycine max), bovine serum albumin (BSA) were purchased from Sigma-Aldrich. 2-O-α-D-Mannopyranosyl-D-mannopyranose (Man2) and 3,6-di-O-(α-D-mannopyranosyl)-D-mannopyranose (Man3) were obtained from V-Labs Inc (Covington, Louisiana). Absolute ethanol (200-proof) was purchased from PHARMCO-AAPER. 3-(Trimethoxysilyl)butyl aldehyde (90%) was obtained from United Chemical Technologies, Inc. All chemicals were used as received without purification. Water used was from a Milli-Q ultrapure water purification system. Dialysis tubes (G-Biosciences Tube-O-dialyzer, 15K, medium) were purchased from VWR International. E. coli ORN178 and ORN208 were grown in the Luria-Bertani Broth medium at 37° C. to an optical density of 0.9 at 600 nm (approximately 109 cells per mL) and precipitated by centrifugation at 3,000 rpm for 4 minutes. Milli-Q water for contact angle measurements as well as for cleaning gold slides and silicon wafer was obtained from a Millipore Milli-Q system with at least 18.2 MΩ resistivity. Concentrated H₂SO₄, H₂O₂(35%), toluene, dichloromethane, and chloroform were purchased from Fisher. Dichloromethane was dried by refluxing in CaH₂ for 3 hours and was distilled before use. Other solvents were used as received. Polystyrene (PS, ca 280,000), poly(ethylene oxide) (PEO, 1,000,000), polyvinylpyrrolidone (PVP, ca 1,300,000), poly(acrylic acid) (PAA, 35 wt. % solution in water), polyethylenimine (PEI, 80% ethoxylated, 35-40 wt. % solution in water), 1,2-poly(vinylbenzyl chloride) (PVBC, 60/40 mixture of 3- and 4-isomers) and tetraethyl orthosilicate (98%) were received from Aldrich. Poly(2-ethyl-2-oxazoline) (PEOX, ca 500,000) were obtained from Alfa Aesar. 1,2-Polybutadiene (PB, ca 100,000) was purchased from Scientific Polymer Products Inc. (Ontario, N.Y.). 3-Aminopropyltriethoxysilane was brought from TCI. Fluorescein isothiocyanate isomer I (FITC), bovine serum albumin (BSA, 96%, ˜66 kDa) and phosphate buffered saline (PBS, pH 7.4) were purchased from Sigma. Ethanol (absolute, anhydrous) was purchased from Pharmco-AAper Inc. (Brookfield, Conn.).

Silicon wafers with a 35 Å native oxide layer were purchased from WaferNet, Inc. (San Jose, Calif.). Glass slides (3 inch×1 inch×1 mm) were obtained from Corning Glass Works, Scientific Glassware Department (Corning, N.Y.). The long-pass optical filter (280-nm) was purchased from Schott Glass Technologies, Inc. (Fullerton, Calif.).

Fluorescence measurements were conducted on a PTI spectrofluorometer (Photon Technology International). TEM images were obtained on a JEOL 100CX transmission electron microscope operating at an accelerating bias voltage of 100 kV. The specimens were prepared by dropping nanoparticles suspensions (10 μL) onto a 200 mesh copper grid (coated with carbon supporting film, Electron Microscopy Sciences). Dynamic light scattering (DLS) experiments were carried out on Horiba LB-550 Dynamic Light Scattering Nano-Analyzer.

The salt HAuCl₄.nH₂O was purchased from Sigma-Aldrich®. UV visible spectra were recorded using a Perkin-Elmer Lambda 45 spectrophotometer. IR spectrums were obtained using a Perkin-Elmer 2000 Fourier transform spectrometer.

Dynamic Light Scattering (DLS) measurements were taken using Horiba LB-550®.

### Example 1

This example concerns the synthesis of FITC-doped silica nanoparticles FSNPs. Fluorescein isothiocyanate (39 mg, 0.10 mmol) was mixed with APTMS (23 μL, 0.10 mmol) in 100 mL of absolute ethanol, and was stirred at 42° C. for 24 hours to yield the FITC-silane precursor as a bright yellow solution. The fluorescent nanoparticles were synthesized following a modified protocol from the classic Stöber protocol, similar to what was previously described. The dye precursor solution (5 mL) was mixed with tetraethylorthosilicate (TEOS) (2.8 mL), and the mixture was added to 200 proof absolute ethanol (34 mL) followed by NH₄OH (25%, 2.8 mL). The reaction was allowed to proceed at room temperature for at least 8 hours with vigorous stirring to yield a bright yellow colloidal solution. The particle diameters were determined by TEM and DLS.

### Example 2

This example concerns the synthesis of exemplary PFPA-silanes, as illustrated in the following synthetic scheme for PFPA-silane 1.

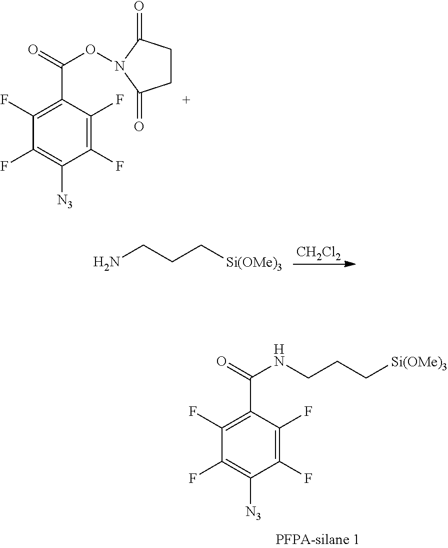

### Synthesis of N-(3-Trimethoxysilylpropyl)-4-azido-2,3,5,6-tetrafluorobenzamide

(1): A solution of N-succinimidyl-4-azidotetrafluorobenzoate2 (102.8 mg, 3.1 mmol), 3-aminopropyltimethoxysilane (65 μL, 3.7 mmol), and CH₂Cl₂ (4 mL) was capped in argon and stirred for 5 hours at 23° C. The mixture was evaporated and approximately 0.3 g of silica gel was added to the dried residue. This sample was purified by column chromatography on silica gel using 1:3 v/v CHCl₃/hexane containing 2% methanol as an eluent. Evaporation of the solvent afforded N-(3-trimethoxysilylpropyl)-4-azido-2,3,5,6-tetrafluorobenzamide as a clear viscous liquid (99.5 mg, 88%). Refractive index: n_{D} 1.503. FTIR (neat, NaCl plate): 3287, 3088, 2946, 2844, 2126, 1661, 1566, 1489, 1271, 1087, 998, 823, 779 cm⁻¹. ¹H NMR (CDCl₃): δ 3.56 (s, 9H), 3.47 (q, J=12 Hz, 2H), 1.76 (m, J=15 Hz, 2H), 1.61 (s, 1H), 0.72 (t, J=16 Hz, 2H). ¹⁹F NMR (CDCl₃): 8-144.0 (br, 2F), −153.8 (br, 2F). UV-vis (in methanol): continuous broad peaks with maxima at 256 and 205 nm, respectively. Anal. Calcd for C₁₃H₁₆N₄F₄O⁴Si: C, 39.39; H, 4.07; N, 14.13. Found: C, 39.34; H, 4.16; N, 13.81.

### Example 3

This example concerns the synthesis of exemplary PFPA-disulfides/thiols as illustrated by the following synthetic scheme.

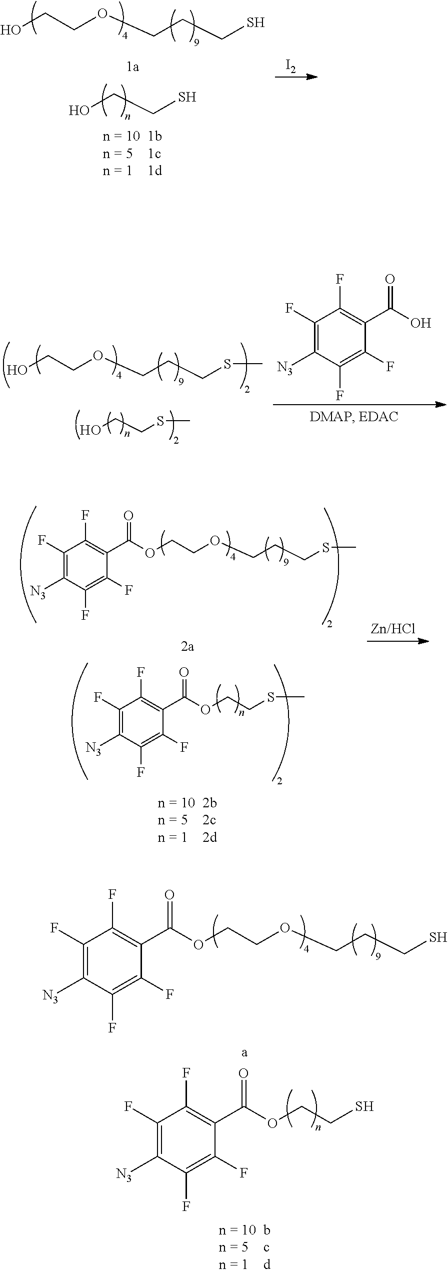

### Synthesis of 3,6,9,12,37,40,43,46-Octaoxa-24,25-dithiaoctatetracontane-1,48-diylbis(4-azido-2,3,5,6-tetrafluorobenzoate) (2a)

(1-Mercaptoundec-11-yl)tetra(ethylene glycol) (1a, 100 mg, 0.26 mmol) in absolute ethanol (10 mL) was titrated with a saturated solution of iodine in ethanol until the brown color of iodine persisted. The solution was concentrated to 2 mL and then water (10 mL) was added. The solution was extracted with diethyl ether (3×10 mL). The combined ethereal extracts were washed with brine, dried over Na₂SO₄, and the solvent was removed under reduced pressure to afford the disulfide as a brown oil. A solution of 4-azido-2,3,5,6-tetrafluorobenzoic acid (61.8 mg, 0.26 mmol) in CH₂Cl₂ (10 mL) was cooled to 0° C., and DMAP (3.7 mg, 0.03 mmol) and EDAC (57.5 mg, 0.3 mmol) were added. The disulfide obtained above was then added, and the solution was stirred for 1 hour, after which the reaction was allowed to warm up to room temperature and was stirred for 12 hours under argon. The product was recovered by extracting with CH₂Cl₂ and the organic layer was washed with water, brine, and dried over Na₂SO₄. The crude product was purified by column chromatography with 1:8 v/v hexanes/ethyl acetate to afford PFPA-disulfide 2a as a clear oil (57.1 mg, 37%). ¹H NMR (400 MHz, CDCl₃): δ (ppm) 4.52 (t, J=3.2 Hz, 4H), 3.81 (t, J=3.2 Hz, 4H), 3.66-3.54 (m, 24H), 3.46 (t, J=5.2 Hz, 4H), 2.67 (t, J=7.2 Hz, 4H), 1.61 (m, 4H), 1.57 (m, 4H), 1.28-1.23 (m, 28H). ¹³C NMR (100 MHz, CDCl₃): δ (ppm) 159.4, 145.4 (d, J=260 Hz), 140.2 (d, J=252 Hz), 127.9, 109.5, 70.4, 69.6, 69.1, 68.4, 61.3, 37.4, 36.7, 30.4, 30.1, 29.3, 29.2, 28.4, 28.0.

### Synthesis of 23-Mercapto-3,6,9,12-tetraoxatricosyl 4-azido-2,3,5,6-tetrafluorobenzoate (a)

To a solution of PFPA-disulfide 2a (40 mg, 0.033 mmol) in 1:1 v/v ethanol/acetonitrile (20 mL), zinc dust (50 mg) and concentrated HCl (0.05 mL) were added, and the reaction mixture was stirred under argon at room temperature for 1 hour. The reaction mixture was filtered, and the solvents were removed by rotary evaporation. The residue was dissolved in chloroform, and the resulting solution was washed twice with water followed by dilute NaHCO3 solution, and was dried over Na₂SO₄. The crude product was purified by column chromatography with 1:8 v/v hexanes/ethyl acetate to afford PFPA-thiol as a clear oil (32.4 mg, 81%). ¹H NMR (400 MHz, CDCl₃): δ (ppm) 4.40 (t, J=2.8 Hz, 2H), 3.75 (t, J=2.6 Hz, 2H), 3.65-3.52 (m, 12H), 3.45 (t, J=5.6 Hz, 2H), 2.40 (q, J=7.6 Hz, 2H), 1.67 (m, 4H) 1.27-1.23 (m, 15H). ¹³C NMR (100 MHz, CDCl₃): δ (ppm) 159.1, 145.3 (d, J=255 Hz), 140.4 (d, J=252 Hz), 128.2, 108.9, 70.5, 69.9, 69.1, 68.5, 61.8, 37.7, 30.3, 29.7, 29.2, 28.7, 28.1, 23.4. JR (film) 2925, 2854, 2129, 1737, 1648, 1488, 1258, 1119, 998 cm⁻¹. HRMS (ESI) C₂₆H₃₉F₄N3O₆S [M+H]+ calcd 598.2574, found 598.2535.

### Synthesis of 11,11′-Disulfanediylbi(undecane-11,1-diyl)bis(4-azido-2,3,5,6-tetrafluorobenzoate) (2b)

Compound 2b was prepared following a previously reported procedure: Wang, X.; Ramström, O.; Yan, M. J. Mater. Chem. 2009, 19, 8944-8949.

### Synthesis of 11-Mercaptoundecyl 4-azido-2,3,5,6-tetrafluorobenzoate (b)

Compound b was synthesized from 2b following the same procedure as described above for a. ¹H NMR (400 MHz, CDCl₃): δ (ppm) 4.36 (t, J=6.6 Hz, 2H), 2.52 (q, J=7.6 Hz, 2H), 1.72 (m, 2H), 1.61 (m, 2H), 1.44-1.20 (m, 15H). ¹³C (100 MHz, CDCl₃) δ (ppm) 159.5, 145.1 (d, J=252 Hz), 140.2 (d, J=256 Hz) 125.3, 108.1, 64.3, 36.1, 29.0, 28.1, 25.4, 23.7. Anal. Calcd for C₁₈H₂₃F₄N₃O₂S: C, 51.30; H, 5.50; N, 9.97. Found C, 51.36; H, 5.51; N, 9.99.

### Synthesis of 6,6′-Disulfanediylbis(hexane-6,1-diyl)bis(4-azido-2,3,5,6-tetrafluoro-benzoate) (2c)

Compound 2c was synthesized from 1c following the same procedure as described above for 2a. ¹H NMR (400 MHz, CDCl₃) δ (ppm) 4.37 (t, J=6.4 Hz, 2H), 2.69 (t, J=7.2 Hz, 2H), 1.81-1.65 (m, 4H), 1.53-1.38 (m, 4H). ¹³C NMR (100 MHz, CDCl₃): δ (ppm) 159.7, 145.1 (d, J=256 Hz), 140.5 (d, J=258 Hz), 124.4, 107.2, 64.8, 39.1, 36.4, 29.1, 28.4, 23.1. Anal. Calcd for C₂₆H₂₄F₈N₆O₄S₂: C, 44.57; H, 3.45; N, 12.00. Found C, 44.63; H, 3.50; N, 11.96.

### Synthesis of 6-Mercaptohexyl 4-azido-2,3,5,6-tetrafluorobenzoate (c)

Compound c was synthesized from 2c following the same procedure as described above for a. ¹H NMR (400 MHz, CDCl₃): δ (ppm) 4.32 (t, J=6.4 Hz, 2H), 2.54 (q, J=7.6 Hz, 2H), 1.75 (m, 2H), 1.64 (m, 2H), 1.45 (m, 4H), (t, J=7.8 Hz, 1H). ¹³C NMR (100 MHz, CDCl₃): δ (ppm) 159.3, 145.2 (d, J=250 Hz), 140.4 (d, J=266 Hz), 123.1, 108.1, 64.2, 36.1, 29.0, 28.1, 25.4, 23.7. Anal. Calcd for C₁₃H₁₃F₄N₃O₂S: C, 44.44; H, 3.73; N, 11.96. Found C, 44.31; H, 3.75; N, 12.04.

### Synthesis of 2,2′-Disulfanediylbis(ethane-2,1-diyl)bis(4-azido-2,3,5,6-tetrafluoro-benzoate) (2d)

Compound 2d was prepared following a previously reported procedure: Pei, Y.; Yu, H.; Pei, Z.; Theurer, M.; Ammer, C.; Andre, S.; Gabius, H. J.; Yan, M.; Ramstrom, O. Anal. Chem. 2007, 79, 6897-6902.

### Synthesis of 2-Mercaptoethyl 4-azido-2,3,5,6-tetrafluorobenzoate (d)

Compound d was synthesized from 2d following the same procedure as described for a. ¹H NMR (400 MHz, CDCl₃): δ (ppm) 4.45 (t, J=6.4 Hz, 2H), 2.86 (q, J=7.2 Hz, 2H), 1.63 (t, J=8.8 Hz, 1H); ¹³C NMR (100 MHz, CDCl₃): δ (ppm) 159.1, 145.3 (d, J=258 Hz), 140.5 (d, J=267 Hz), 123.7, 107.2, 64.0, 23.3. Anal. Calcd for C₉H₅F₄N₃O₂S: C, 36.62; H, 1.71; N, 14.23. Found C, 36.25; H, 1.77; N, 14.02.

### Example 4

This example concerns the synthesis of exemplary PFPA-phosphates as illustrated by the following synthetic scheme.

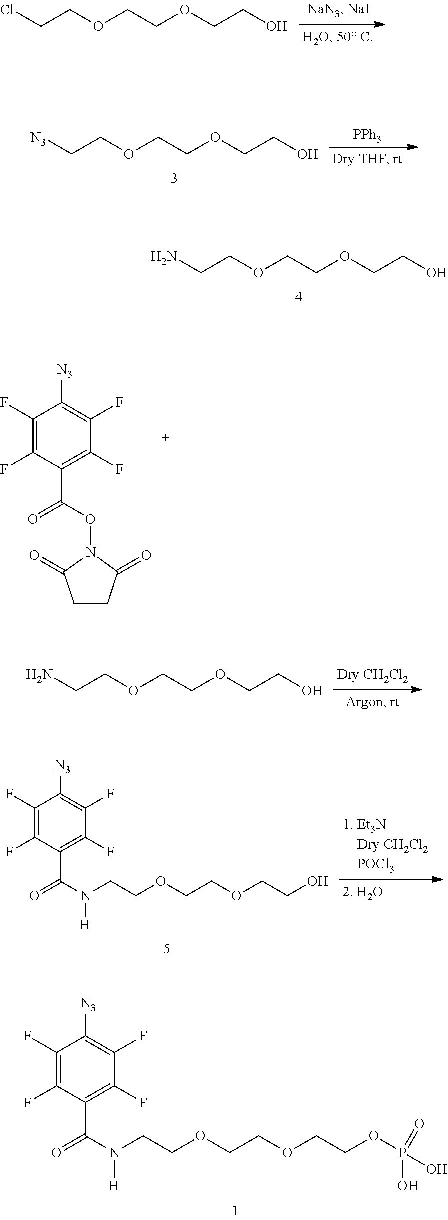

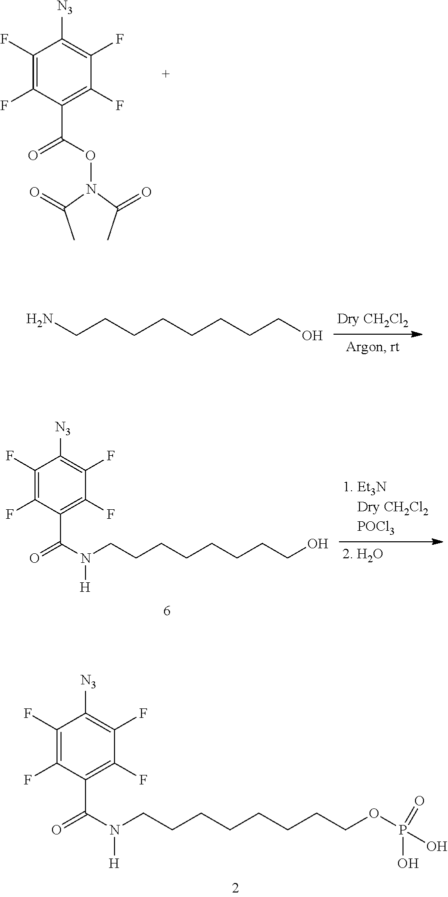 [SMILES: CC(=O)N(OC(=O)c1c(F)c(F)c(N=[N+]=[N-])c(F)c1F)C(C)=O ; "+" ; NCCCCCCCCO ; [N-]=[N+]=Nc1c(F)c(F)c(C(=O)NCCCCCCCCO)c(F)c1F ; [N-]=[N+]=Nc1c(F)c(F)c(C(=O)NCCCCCCCCOP(=O)(O)O)c(F)c1F]

### Synthesis of 2-(2′-(2″-(4-Azidoethoxy)ethoxy)ethanol (3)

2-(2′-(2″-Chloroethoxy)ethoxy)ethanol (0.5 g, 2.97 mmol), NaI (0.09 g, 0.6 mmol), and NaN₃ (2.0 g, 30 mmol) were dissolved in water (10 mL), and the solution was stirred at 50° C. for 48 hours. The reaction mixture was then filtered, and the aqueous phase was extracted with ethyl acetate (4×10 mL). The organic extracts were collected and evaporated to give 3 as a light yellow liquid (0.4 g, 77%). ¹H NMR (400 MHz, CDCl₃, δ): 3.72 (t, 2H, J=5.0 Hz), 3.68 (m, 6H), 3.60 (t, 2H, J=5.0 Hz), 3.40 (t, 2H, J=5.0 Hz), 3.19 (br, 1H). ¹³C NMR (100 MHz, CDCl₃, 5): 72.63, 70.59, 70.34, 69.98, 61.56, 50.61.

### Synthesis of 2-(2′-(2″-Aminoethoxy)ethoxy)ethanol

(4) 2-(2′-(2″-(4-Azidoethoxy)ethoxy)ethanol (3) (400 mg, 2.4 mmol) was dissolved in dry THF (10 mL), and the solution was cooled to 0° C. Triphenyl phosphine (600 mg, 2.4 mmol) was added, and the reaction mixture was stirred at room temperature for 10 hours. Water (0.12 mL, 6.7 mmol) was added, and the mixture was stirred for another 5 hours. The reaction mixture was diluted with water and washed with toluene (3×10 mL). The aqueous layers were collected and evaporated to give 4 as a colorless liquid (240 mg, 70% crude). This compound was used directly in the next step without further purification.

### Synthesis of 4-Azido-2,3,5,6-tetrafluoro-N-(2-(2′-(2″-hydroxyethoxy)ethoxy)ethyl)benzamide (5)

Dry CH₂Cl₂ (10 mL) was added to a mixture of N-succinimidyl 4-azidotetrafluorobenzoate (400 mg, 1.2 mmol) and 2-(2′-(2″-aminoethoxy)ethoxy)ethanol (4) (180 mg, 1.2 mmol) via syringe. The mixture was purged with argon for 30 minutes and stirred at room temperature for 6 hours. Water (10 mL) was added, and the mixture was extracted by CHCl₃ (3×10 mL). The organic layers were combined, dried over anhydrous Na₂SO₄, and evaporated in vacuo. The residual was purified by column chromatography (10/4/0.5, ethylacetate/hexanes/methanol) to give 5 as a white waxy solid (180 mg, 41%). ¹H NMR (400 MHz, CDCl₃, δ): 7.04 (1H), 3.65 (m, 10H), 3.55 (t, 2H, J=5.0 Hz), 2.69 (br, 1H). ¹³C NMR (100 MHz, CDCl₃, 5): 157.93, 145.28, 142.78, 141.74, 139.08, 121.60, 111.85, 72.47, 70.28, 70.23, 69.47, 61.51, 39.99. Anal. Calcd. for C₁₃H₁₄F₄N₄O₄: C, 42.63; H, 3.85; N, 15.30. Found: C, 42.43; H, 3.87; N, 15.27.

### Synthesis of 2-(2′-(2″-(4-Azido-2,3,5,6-tetrafluorobenzamido)ethoxy)ethoxy)ethyl Dihydrogen Phosphate (1)

4-Azido-2,3,5,6-tetrafluoro-N-(2-(2′-(2″-hydroxyethoxy)ethoxy)ethyl)benzamide (5) (180 mg, 0.49 mol) was dissolved in dry CH₂Cl₂, and the solution was purged with argon. Anhydrous triethylamine (0.1 mL, 0.7 mmol) was added, and the mixture was cooled to 0° C. with an ice bath before POCl₃ (0.06 mL, 0.7 mmol) was added dropwise. The reaction mixture was allowed to warm to room temperature and stirred for another 6 hours. Water (10 mL) was added, and the mixture was stirred for 1 hour. The reaction mixture was extracted with CHCl₃ (3×10 mL). The organic layers were collected, dried over anhydrous Na₂SO₄, and evaporated to give 1 as a light yellow, highly viscous clear liquid (120 mg, 55%). ¹H NMR (400 MHz, CDCl₃, δ): 6.94 (br, 1H), 4.44 (m, 2H), 3.80 (m, 2H), 3.67 (m, 8H). ¹³C NMR (100 MHz, CDCl₃, δ): 157.94, 145.30, 142.80, 141.76, 139.09, 121.63, 111.82, 71.03, 70.77, 70.32, 69.43, 68.97, 40.01. Anal. Calcd. for C₁₃H₁₅F₄NO₇P: C, 34.99; H, 3.39; N, 12.56. Found: C, 34.72; H, 3.18; N, 12.23.

### Synthesis of 4-Azido-2,3,5,6-tetrafluoro-N-(8-hydroxyoctyl)benzamide (6)

N-Succinimidyl 4-azidotetrafluoro benzoate (200 mg, 0.6 mmol) and 8-aminooctan-1-ol (90 mg, 0.6 mmol) were added to dry CH₂Cl₂ (10 mL), and the solution was purged with argon for 30 min and stirred at room temperature for 6 hours. Water (10 mL) was then added, and the mixture was extracted with CHCl₃ (3×10 mL). The organic layers were combined, dried over anhydrous Na₂SO₄, and evaporated in vacuo. The residual was purified by column chromatography (10/4/0.5, ethylacetate/hexanes/methanol) to give 6 as a white power (110 mg, 50%). ¹H NMR (400 MHz, CDCl₃, 5): 5.96 (s, 1H), 3.64 (t, 2H, J=7.0 Hz), 3.45 (q, 2H, J=7.0 Hz), 1.59 (m, 4H), 1.35 (m, 8H). ¹³C NMR (100 MHz, CDCl₃, 5): 157.59, 145.34, 142.79, 141.63, 139.15, 121.82, 111.61, 62.99, 40.33, 32.70, 29.27, 29.23, 29.10, 26.65, 25.61. Anal. Calcd. for C₁₅H₁₈F₄N₄O₂: C, 49.72; H, 5.01; N, 15.46. Found: C, 49.63; H, 5.18; N, 15.47

### Synthesis of 8-(4-Azido-2,3,5,6-tetrafluorobenzamido)octyl dihydrogen phosphate (2)

4-Azido-2,3,5,6-tetrafluoro-N-(8-hydroxyoctyl)benzamide (6) (250 mg, 0.69 mmol) was dissolved in dry THF (10 mL), and the solution was purged with argon. Anhydrous triethylamine (0.13 mL, 0.9 mmol) was added, and the mixture was cooled to 0° C. with an ice bath before POCl₃ (0.08 mL, 0.9 mmol) was added dropwise. The reaction mixture was allowed to warm to room temperature and stirred for another 6 hours. Water (10 mL) was added, and the mixture was stirred for another 1 hour. The reaction mixture was extracted with CHCl₃ (3×10 mL). The organic layers were collected, dried over anhydrous Na₂SO₄, and was evaporated to give 2 as a light amber viscous liquid (140 mg, 45%). ¹H NMR (400 MHz, CDCl₃, 5): 7.01 (s, 1H), 4.33 (m, 2H), 3.39 (q, 2H, J=7.0 Hz), 1.81 (m, 2H), 1.59 (t, 2H, J=7.0 Hz), 1.36 (m, 8H). ¹³C NMR (100 MHz, CDCl₃, 5): 158.05, 145.13, 142.63, 141.69, 139.04, 121.61, 111.73, 72.57, 40.31, 29.42, 29.04, 28.91, 28.76, 26.54, 25.11.

### Example 5

This example concerns functionalization of silica nanoparticles with PFPA. PFPA-silane (80 mg) was synthesized following a previously reported procedure, M. Yan and J. Ren, Chem. Mater., 2004, 16, 1627-1632, was added directly to the Stöber solution prepared above. The mixture was stirred at room temperature overnight. The next day the mixture was brought to reflux while continuing stirring for 1 hour at approximately 78° C. to facilitate the covalent bond formation between PFPA-silane and the silica nanoparticles. P. Gann and M. Yan, Langmuir, 2008, 24, 5319-5323. The mixture was centrifuged at 8,000 rpm for 10 minutes, and the precipitate was redispersed in the fresh solvent by sonication. This centrifugation/redispersion procedure was repeated three times with ethanol and twice with acetone.

### Example 6

This example concerns conjugation of carbohydrates onto FSNPs. Carbohydrates were coupled to gold nanoparticles, using the method reported in X. Wang, O. Ramstrom and M. Yan, J. Mater. Chem., 2009, 19, 8944-8949. The solution of PFPA-functionalized FSNPs in acetone (20 mg/mL, 5 mL) was placed in a flat-bottom dish, and an aqueous solution of carbohydrate (10 mg/mL, 1 mL) was added. The mixture was covered with a 280-nm long-path optical filter (WG-280, Schott Glass) and was irradiated with a 450-W medium pressure Hg lamp (Hanovia) for 10 minutes under vigorous stirring. Centrifugation of the solution at 8,000 rpm for 10 minutes separated the carbohydrate-attached FSNPs as precipitates. Excess carbohydrate was removed by membrane dialysis in water for 24 hours. The concentration of FSNP, ˜17.2 mg/mL, was determined by drying the solution under reduced pressure for 3 hours and weighing.

### Example 7

This example concerns determination of carbohydrate density on glyco-FSNPs. A previously developed colorimetric method was followed to determine the density of carbohydrates immobilized on FSNPs (X. Wang, O. Ramstrom and M. Yan, J. Mater. Chem., 2009, 19, 8944-8949). Calibration curves were first obtained for each carbohydrate where carbohydrate solutions of various concentrations were incubated with anthrone/sulfuric acid and the absorbances at 620 nm were measured. A freshly-prepared anthrone solution in concentrated H2SO4 (0.5 wt %, 1 mL) was added to a carbohydrate solution in water (0.5 mL) in an ice bath with stirring. The solution was then heated to 100° C. and stirred for 10 minutes. After being cooled to room temperature, the UV-vis spectra of the resulting solutions were recorded on a Perkin-Elmer Lambda 45 UV-vis spectrometer.

Carbohydrates coupled on nanoparticles were subjected to the same assay where solutions of the glyco-FSNPs in Milli-Q water (30-50 μg/0.5 mL) were treated with anthrone/H₂SO₄. Background absorption due to FSNPs themselves was accounted for by treating FSNPs solution of the same concentration with anthrone/H₂SO₄, and the absorbance at 620 nm was subtracted from that of the glyco-FSNPs. The amount of surface-bound carbohydrate was then computed from the corresponding calibration curve.

### Example 8

This example concerns binding with Con A and E. coli. The binding affinity of FSNP-labeled Man, FSNP-Man, was evaluated using Con A and E. coli strain ORN178 and ORN208 according to the following procedure. FSNP-Man (2.5 mg) were incubated in a pH 7.2 HEPES buffer solution (1 mL, 10 mM) containing 3% BSA for 30 minutes, centrifuged, and the particles were incubated in the pH 7.2 HEPES solution without BSA for another 20 minutes. The nanoparticles were subsequently treated with a solution of Con A in HEPES buffer (1 mL, 10 μg/mL) containing MnCl₂ (1 mM) and CaCl₂ (1 mM), or E. coli solution for 1 hour while shaking. In cases where aggregation was induced after binding with Con A, the suspension was transferred to a centrifuge tube and was centrifuged at 8,000 rpm for 10 min.

### Example 9

This example concerns fabrication of lectin microarrays. Aldehyde-coated glass slides were prepared following a reported procedure: G. MacBeath and S. L. Schreiber, Science, 2000, 289, 1760763. Piranha-cleaned glass slides were treated with a solution of 3-(trimethoxysilyl)butyl aldehyde in toluene (2 mM) for 4 hours, rinsed with toluene and dried with N₂. Solutions of lectins were prepared in pH 7.4 phosphate-buffered saline (PBS) at varying concentrations of 0.1-1 mg/mL with 40% glycerol added to prevent evaporation of the liquid droplets after printing. Con A and SBA were then printed onto the aldehyde-functionalized glass slide using a robotic printer (BioOdyssey Calligrapher miniarrayer; Bio-Rad Laboratories, Inc.). The glass slides were then incubated in a humid chamber (80% humidity) at 25° C. for 3 hours to facilitate the immobilization of the lectins. After incubation, a blocking solution of BSA in pH 7.4 PBS buffer (1%) was added and the slides were incubated for 1 hour, rinsed with the PBS buffer and was dried with N₂.

### Example 10

This example concerns microarray assay and fluorescence imaging. The lectin microarrays were incubated in the solution of glyco FSNPs in HEPES (2.5 mg/mL) for 2 hours, and were then gently rinsed with the HEPES buffer 3 times and dried. The slides were scanned under a microarray scanner (GenePix 4000B, Molecular Devices, Inc) at excitation of 532 nm. The fluorescent images were recorded and the data were analyzed using the supplied software (Axon GenePix Pro 5.1).

### Example 11

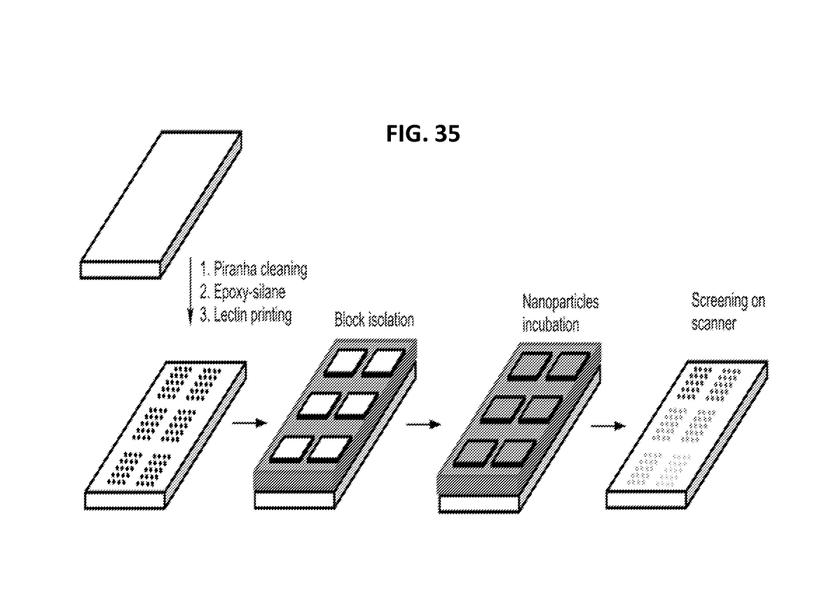

This example concerns fabrication of lectin microarrays as illustrated schematically in FIG. 35. Epoxy-coated glass slides were used to prepare lectin microarrays. Piranha-cleaned glass slides were treated with a solution of 3-glycidyloxypropyltrimethoxysilane (>95%, TCI America, Portland, Oreg.) in toluene (12.6 mM) for 4 hours, rinsed with toluene and dried with N₂. Solutions of lectins, including Con A (Concanavalin A), BSA (Bovine serum albumin), SBA (Soybean Agglutinin), PNA (Peanut Agglutinin), BS-I (Bandeiraea simplicifolia) purchased from Sigma-Aldrich (St. Louis, Mo.); and DBA (Dolichos Biflorus Agglutinin), UEA (Ulex Europaeus Agglutinin I), WGA (Wheat Germ Agglutinin) and RCA (Ricinus Communis Agglutinin I) purchased from Vector Laboratories (Burlingame, Calif.), were prepared in phosphate-buffered saline at concentrations of 1 mg/mL (except PNA and RCA at 0.25 mg/mL) with 40% glycerol added to prevent evaporation of the liquid droplets after printing. CV-N (Cyanovirin-N) and OAA (Oscillatoria Agardhii Agglutinin) wild-type lectin and mutants are obtained from Dr. Angela M. Gronenborn's research laboratory at University of Pittsburgh. Lectins were spotted onto the epoxy-coated glass slide using a robotic printer (BioOdyssey Calligrapher Mini-arrayer; Bio-Rad Laboratories, Inc.). The glass slides were then incubated in a humid chamber (80% humidity) at 25° C. for 3 hours to immobilize the lectins. After incubation, blocking solution of BSA in pH 7.4 PBS buffer (1%) was added and the slides were incubated for 1 hour, rinsed with the PBS buffer and dried with nitrogen gas.

### Example 12

This example concerns microarray assay and fluorescence imaging. A SecureSeal™ hybridization chambers sheet (Grace Bio-lab, Bend, Oreg.) was attached on a glass slide to create 16 individual wells, and the nanoparticle solution (0.1 mL, 0.5 mg/mL) was added into each well followed by 1 hour incubation with gentle shaking. For competition binding assays, carbohydrate-conjugated FSNP (0.5 mg/mL) were mixed with various concentrations of free ligands (10⁻⁴ mM-10⁵ mM), and added to each well on lectin microarray. After rinsing with fresh buffer three times and once with Milli-Q water, the slides were dried and scanned under a microarray scanner (GenePix 4100A, Molecular Devices, Inc.) at 532 nm. The fluorescent images were recorded and data were analyzed using software Axon GenePix Pro 5.1.

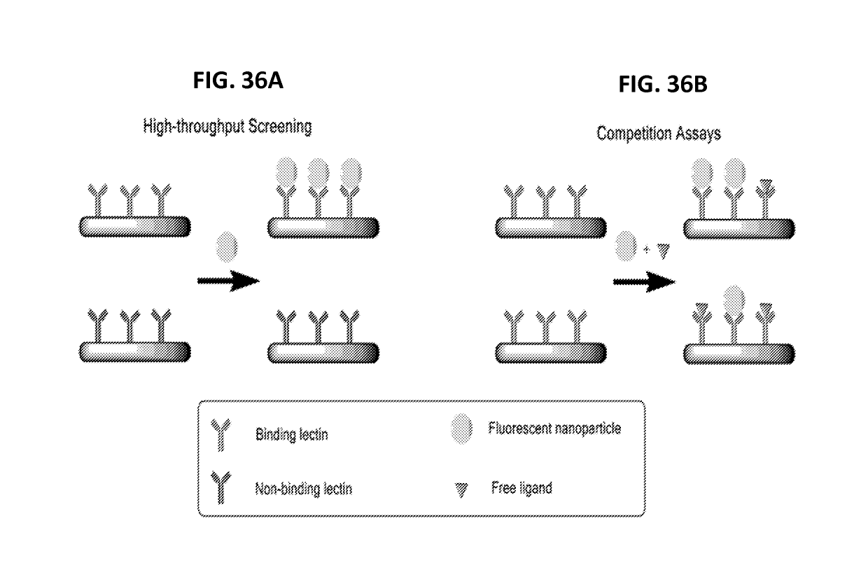

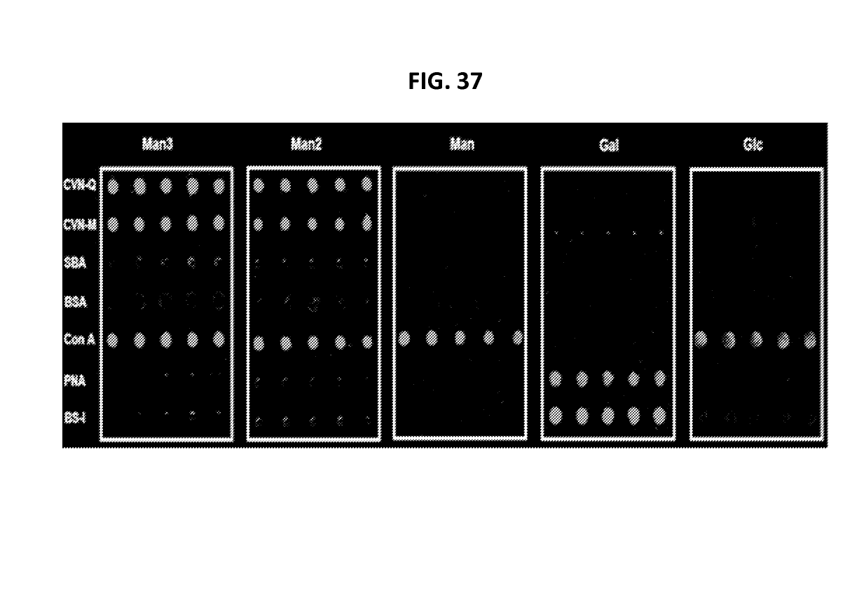

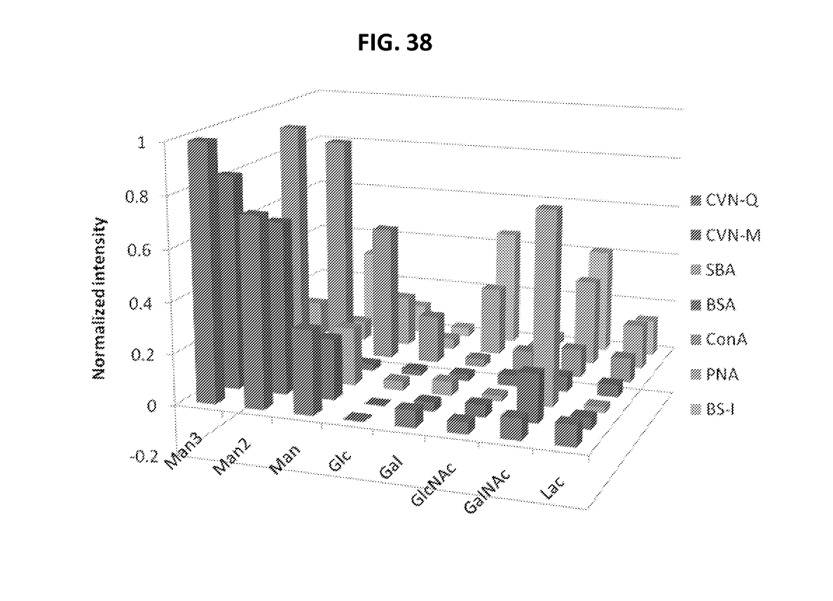

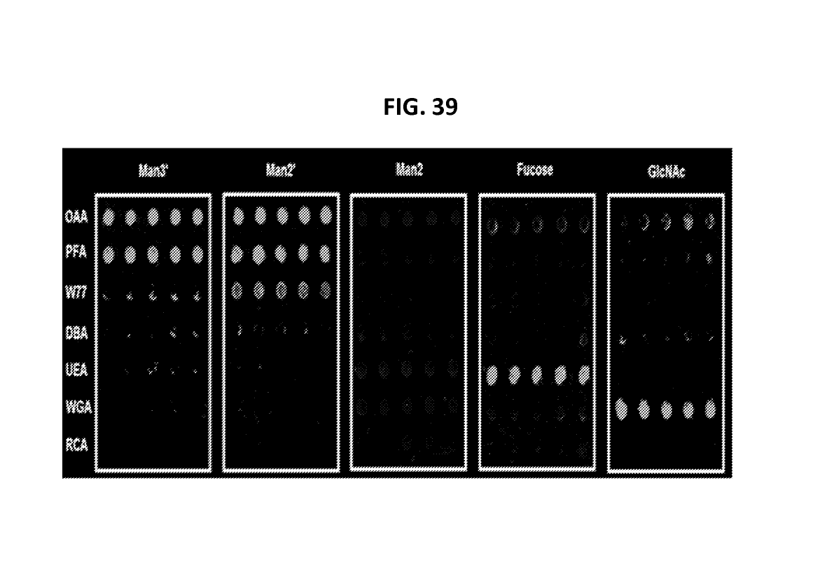

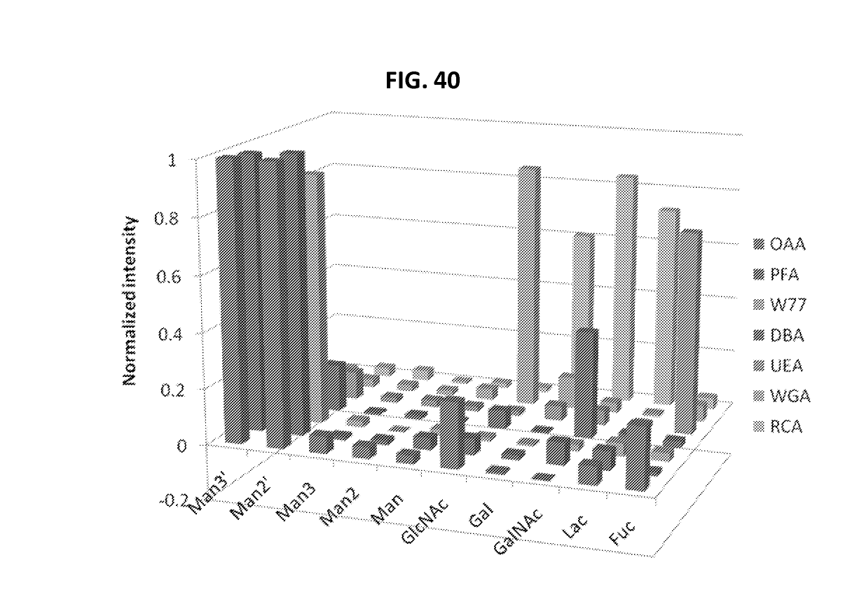

The binding pairs used for Examples 11 and 12 are presented below in Tables 8 and 9, and FIGS. 36-39.

TABLE 8
The lectins and corresponding binding glycan in Lectin Group 1

| No | Lectin | | Origin | Binding glycan |
|---|---|---|---|---|
| 1 | CVN- | Cyanovirin-N | Cyanobacteria | α-1,2-Man |
| | Q | (Q50C) | | |
| 2 | CVN- | Cyanovirin-N | Cyanobacteria | a-1,2-Man |
| | M | (MutDB) | | |
| 3 | SBA | Soybean | Glycine max | GalNAc |
| | | Agglutinin | (soybean) | |
| 4 | BSA | Bovine serum | Bovine serum | N/A |
| | | albumin | | |
| 5 | ConA | Concanavalin A | Canavalia ensiformis | Man |
| 6 | PNA | Peanut | Arachis hypogaea | Gal(β- |
| | | Agglutinin | (peanut) | 1,3)GalNAc/ |
| | | | | Gal |
| 7 | BS-I | Bandeiraea | Griffonia (Bandeiraea) | GalNAc, and |
| | | simplicifolia | simplicifolia seeds | Gal |

TABLE 9
The lectins and corresponding binding glycan in Lectin Group 2

| No | Lectin | | Origin | Binding ligands |
|---|---|---|---|---|
| 1 | OAA | Oscillatoria Agardhii | Cyanobacteria | α-1,6-Man |
| | | Agglutinin | | |
| 2 | PFA | Homolog of OAA | N/A | α-1 ,6-Man |
| 3 | W77 | Mutant of OAA | Cyanobacteria | α-1,6-Man |
| 4 | DBA | Dolichos Biflorus | Dolichos biflorus | GalNAc |
| | | Agglutinin | (horse gram) seeds | |
| 5 | UEA | Ulex Europaeus | Ulex europaeus | L-fucose |
| | | Agglutinin I | (Furze gorse) seed | |
| 6 | WGA | Wheat Germ | Triticum vulgaris | GlcNAc |
| | | Agglutinin | (wheat germ) | |
| 7 | RCA | Ricinus Communis | Ricinus communis | Gal/GalNAc/ |
| | | Agglutinin I | (castor bean) | Lac |
| | | | seeds | |

### Example 13

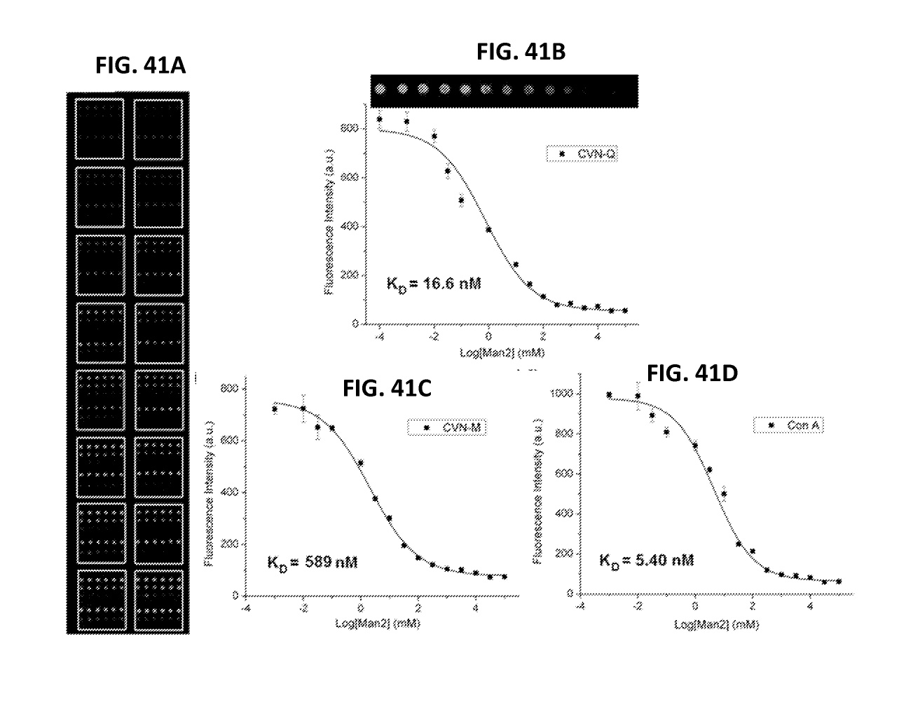

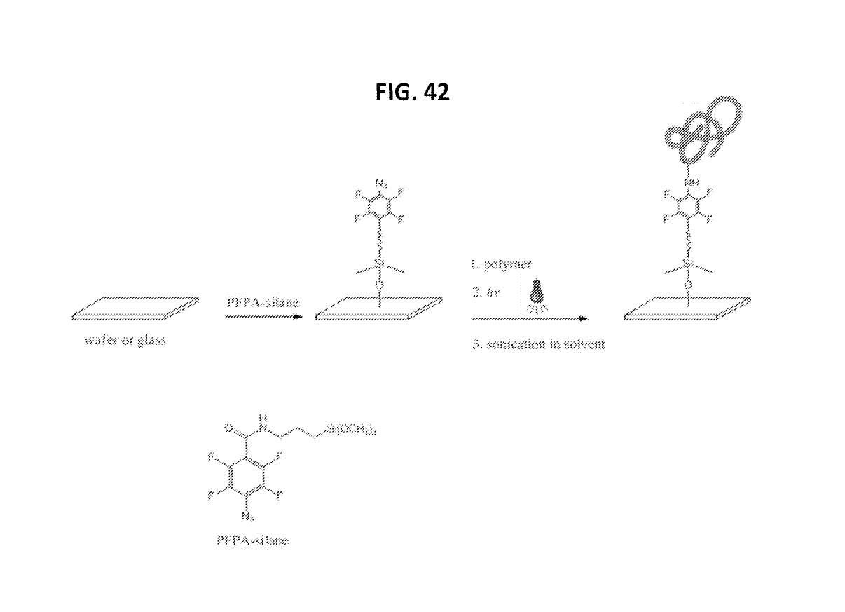

This example concerns fabrication of polymer arrays on silicon wafers or glass slides, as illustrated schematically in FIG. 41. Silicon wafers or glass slides were cut into 1×1 inch pieces, cleaned using piranha solution (3:1 v/v conc. H₂SO₄/H₂O₂) at 80-90° C. for 1 hour (caution: the piranha solution reacts vigorously with organic solvents.), washed in boiling water three times for 60 minutes each, and then dried carefully under a stream of nitrogen. The cleaned wafers or glass slides were soaked in a solution of PFPA-silane in toluene (12.6 mM) for 4 hours, rinsed with toluene, and dried under nitrogen. The wafers or glass slides were allowed to cure at room temperature for 24 hours.

Polymer arrays were generated by manually spotting solutions of polymers onto the wafers or glass slides using a pipette tip. The printed wafers or glass slides were dried under the ambient condition at room temperature for 30 minutes, and then irradiated with a medium pressure Hg lamp (450 W, Hanovia Ltd.) for 9 minutes. The lamp reached its full power after about a 2.5 minute warm-up to an intensity of 3.5 mW/cm2 at 18 cm from the source as measured by an OAI 306 UV power meter (Optical Associates Inc. Milpitas, Calif.) with a 260-nm sensor. A 280-nm optical filter was placed on the film surface during irradiation. The wafers or glass slides were sonicated in chloroform followed by Milli-Q water for 5 minutes each using a Branson 1510 sonicator (Fisher), and finally dried under nitrogen.

As a comparison, BSA solutions in PBS and Milli-Q water were also immobilized onto the surface via the same procedure.

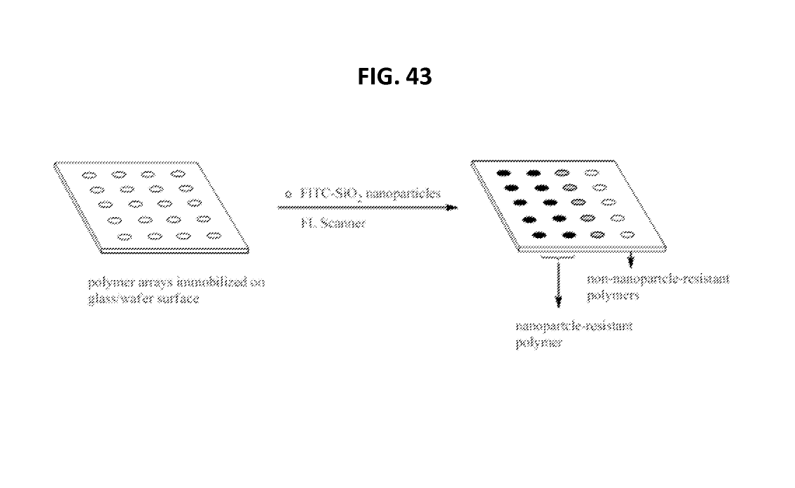

Polymer arrays treated with FITC-doped silica nanoparticles were subjected to fluorescence imaging, as illustrated schematically in FIG. 43. FITC-doped silica nanoparticles were in a pH 7.4 PBS buffer (0.01 M) were prepared. The wafers or glass slides containing the polymer array were incubated in the FITC-doped silica nanoparticle buffer for 1 hour, and washed with Milli-Q water for 3×10 minutes. The wafers or glass slides were finally dried with nitrogen, and imaged immediately by a fluorescence array scanner (GenePix 4000B, Axon Instruments Inc., Foster City, Calif.) at 635 nm excitation and 532 nm emission.

As a comparison, a piece of wafer containing the polymer array was treated with 3% BSA in PBS for 2 hours, washed with Di-water for 3×10 minutes, and then treated with FITC-doped silica nanoparticles using the same procedure mentioned above.

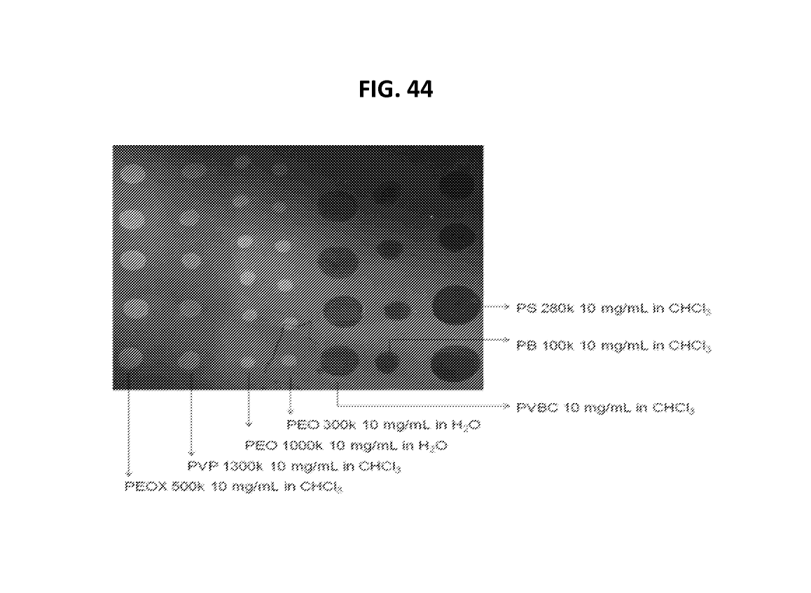

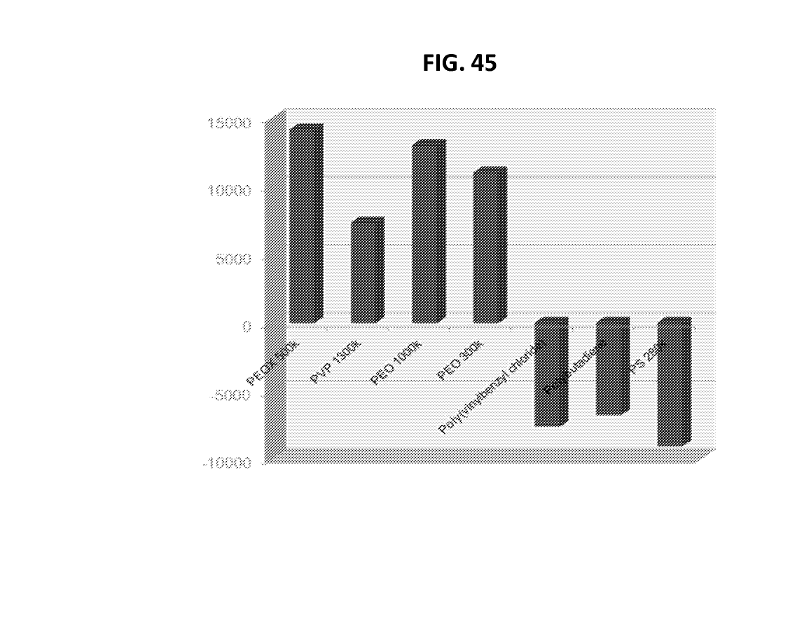

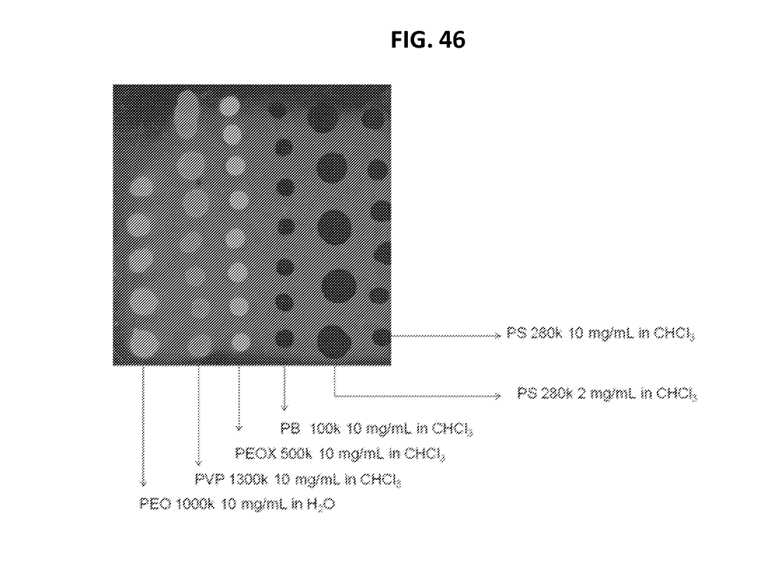

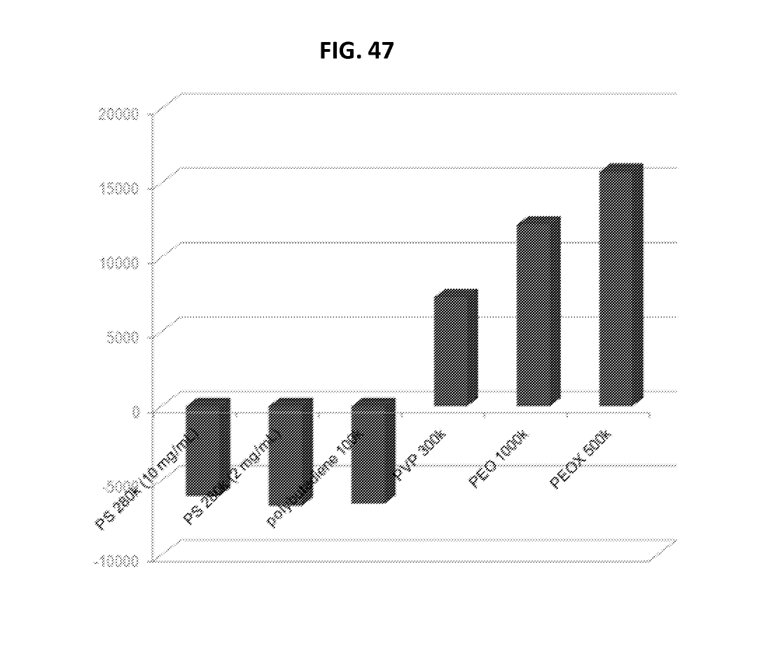

The silica nanoparticle-resistant property of different polymer films was evaluated by fluorescence imaging. Polymers were dissolved in chloroform or Milli-Q water, and the solutions were spotted on the PFPA-functionalized glass slide using a micropipettor tip. In addition to polymers, PBS, a protein which is widely used as a nanoparticle-blocking reagent, was also spotted on a wafer as a reference. The polymers and BSA were immobilized on the wafer or glass surface by UV irradiation. The wafers or glass slides containing the polymer array was then treated with FITC-doped silica nanoparticles in PBS buffer (pH 7.4), washed with water, and the dried wafers or glass slides were analyzed by a microarray scanner. Most hydrophilic polymer spots, such as PEO, PEOX, PVP and PEI, showed bright fluorescence. This indicated that the silica nanoparticles were efficiently adsorbed on these polymeric materials. On the other hand, the fluorescence intensity on the surface of hydrophobic polymer spots, such as PS, PB, PVBC, were significantly lower (FIGS. 44-49).

The results were expected because the surface of silica nanoparticle was known to be hydrophilic. Therefore, hydrophobic surfaces showed higher silica nanoparticle-resistant properties. The only exception was PAA, a highly hydrophilic polymer which also showed a low silica nanoparticle adsorption. This may because the negative charges on the PAA surface at pH 7.4 repelled the silica nanoparticles.

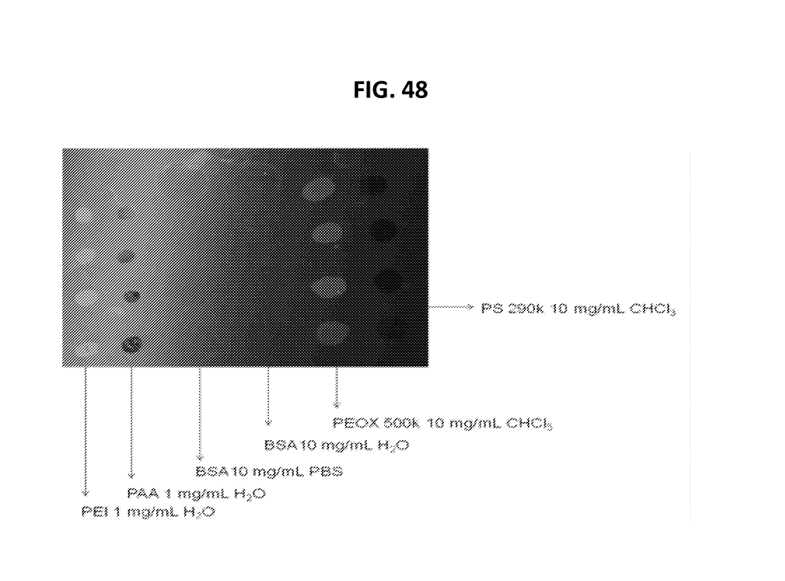

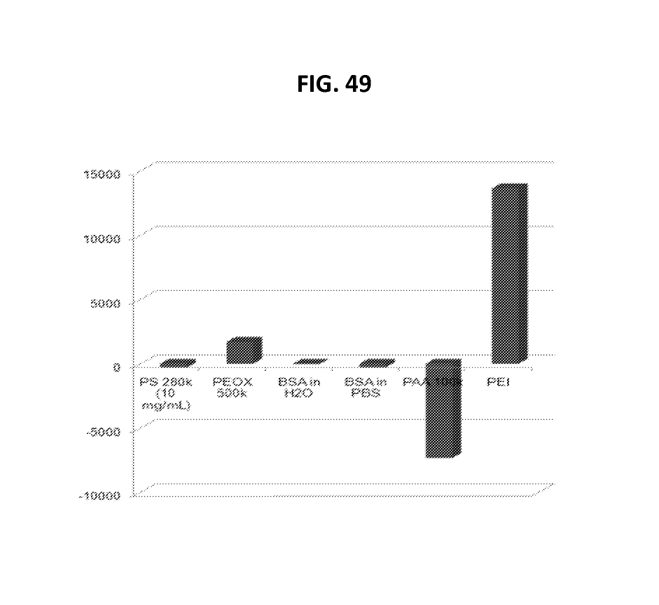

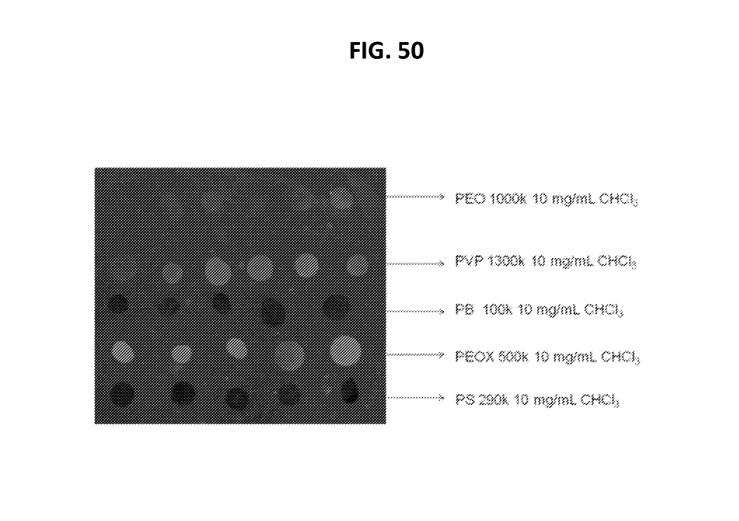

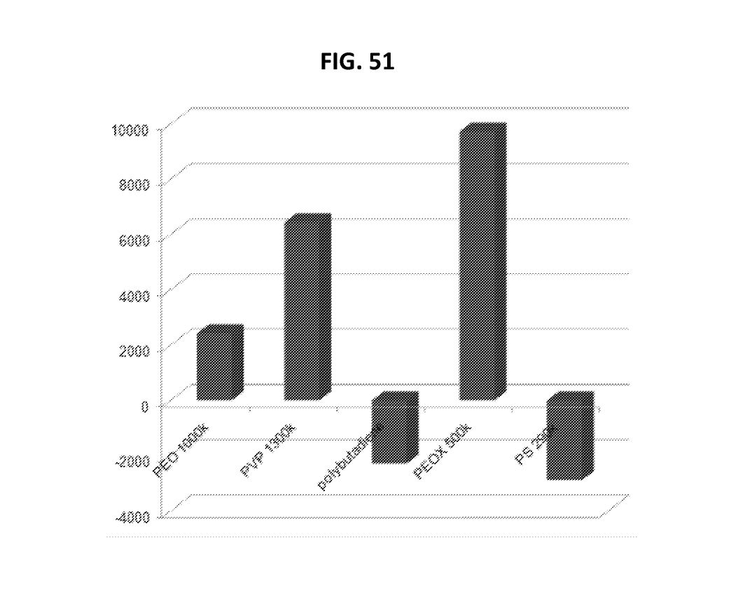

The BSA immobilized on the wafer surface did not show low silica nanoparticle adsorption (FIGS. 48-49). In addition, after blocking with BSA solution, the hydrophilic polymer spots still showed high silica nanoparticle adsorption (FIGS. 50-51).

### Example 14

This example concerns functionalization of silica nanoparticles with epoxy silane. 18 mg 3-glycidyloxytrimethoxy silane (GOPTS) was added directly to 10 ml of a Stöber solution prepared as described above. The mixture was stirred at room temperature overnight, followed by continued stirring at 50° C. for 1 hour. The epoxy functionalized FSNPs were isolated by centrifugation and then redispersed in fresh solvent by sonication. This centrifugation/redispersion procedure was repeated three times with ethanol and twice with HEPES (pH 7.5).

### Example 15

This example concerns the formation of ConA-FSNPs. A 3 ml solution of epoxy-FSNPs solution prepared as described above was added into 3 ml of a 3.33 mg/ml solution of ConA in HEPES (pH 7.5). The mixture was stirred for 3 hours. ConA-FSNPs were washed 3 times with HEPES containing 1 mM CaCl₂ and 1 mM MnCl₂.

### Example 16

This example concerns the synthesis of PFPA-polymer. 10.0 mg (0.107 mmol) PAAm.HCl, 25 mg K₂CO₃ were dissolved in 2 ml of water. 2 ml of a 4.44 mg/ml PFPA-NHS solution in ethanol (10 mg in 2.25 ml ethanol short time ultrasound to dissolve completely) were added. The mixture was vigorously stirred over night to yield a stock solution of 2.5 mg/ml (based on PAAm).

### Example 17

This example concerns the fabrication of carbohydrate microarrays. Piranha-cleaned silica wafers were treated with a solution of GOPTS in toluene (12.6 mM) for 4 hours, rinsed with toluene and dried with N₂. The slides were then reacted with PAAm-4-PFPA solution at 50° C. for 5 hours, and sonicated twice in H₂O for 5 minutes each time. Solutions of carbohydrates were prepared in H₂O. Carbohydrates were then printed onto the PFPA-polymer functionalized slide using a robotic printer (BioOdyssey Calligrapher miniarrayer; Bio-Rad Laboratories, Inc.). The slides were dried under oil pump for 1 hour. A solution of PS or PEO in CHCl₃ (5 mg/ml) was spinning-coated on the slides. The Slides were then covered with a 280-nm long-path optical filter (WG-280, Schott Glass), irradiated with a 450-W medium pressure Hg lamp (Hanovia) for 9 min, sonicated 5 min in CHCl₃ and 5 minutes in H₂O.

### Example 18

This example concerns microarray assay and fluorescence imaging. Carbohydrate microarrays were incubated in a 0.5 mg/ml solution of ConA-FSNPs in HEPES (pH 7.5, 1 mM CaCl₂, 1 mM MnCl₂) for 1 hour, rinsed with HEPES buffer 3 times and dried. The slides were scanned using a Genepix 4000B microarray scanner at excitation of 532 nm. Image analysis was carried out with Axon Genepix Pro 5.1 analysis software (Molecular Devices Corporation, Union City, Calif.).

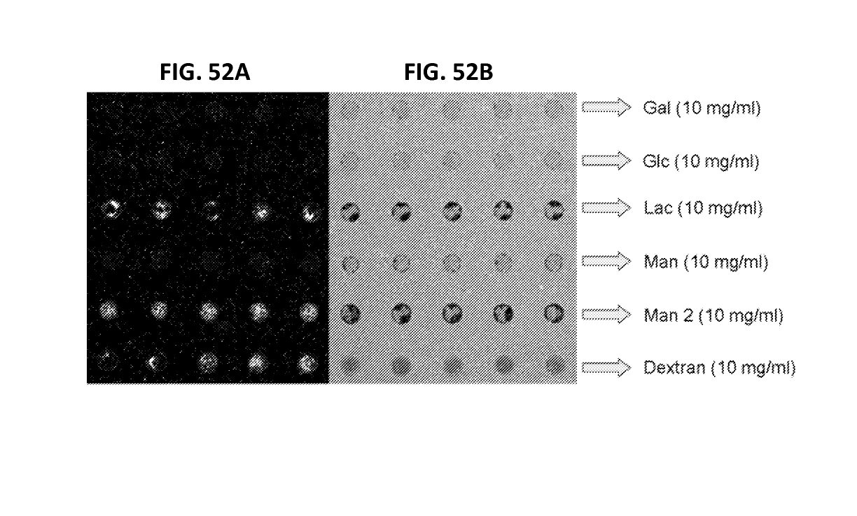

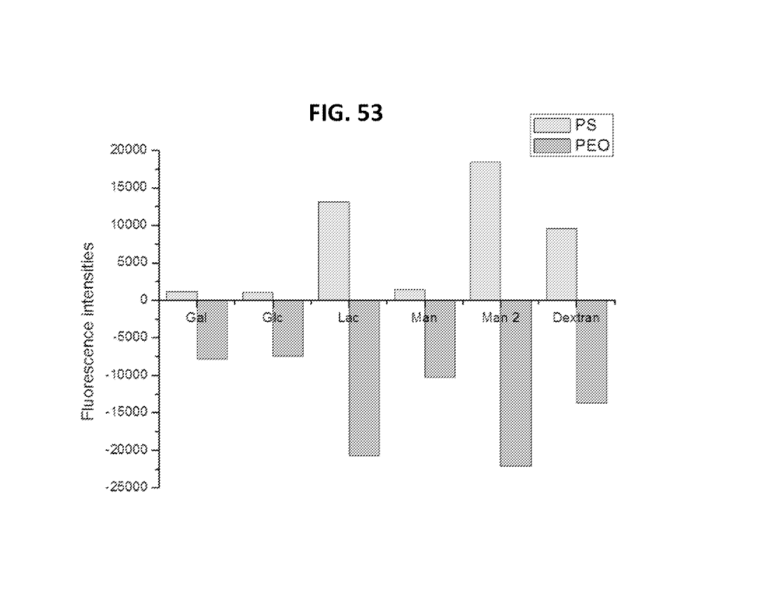

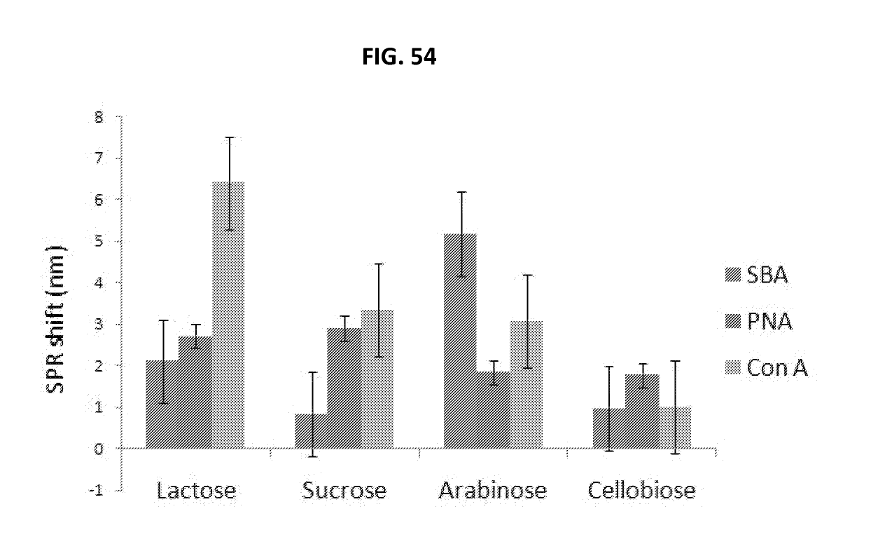

Results obtained for Examples 12-16 are provided in FIGS. 52-53. FIG. 52 provides the fluorescence images of carbohydrate microarrays with PS or PEO coating incubated with ConA-FSNPs at 0.5 mg/ml. For microarrays having a PS coating, there is considerable signal for specific test carbohydrates: ConA-FSNPs label Man 2 most intensely, Dextran and Lac slightly less, and Man, Glc, Gal significantly less. However, for carbohydrate microarrays having a PEO coating, the signal from those “high-intensity” carbohydrate targets was negative (FIG. 53). This indicates that PS coating effectively reduced the non-specific absorption of ConA-FSNPs on the carbohydrate microarrays.

### Example 19

This example concerns determination of immobilized carbohydrate density and coupling yield.

### Example 20

### Synthesis of Perfluorophenylazide (PFPA) disulfide

Synthesis procedure of PFPA disulfide 11,11′-Disulfanediylbis(undecane-11,1-diyl)bis(4-azido-2,3,5,6-tetrafluorobenzoate) was obtained from previously reported procedure from of Wang, X.; Ramstrom, O.; Yan, M. J. Mat. Chem. 2009, 19, 8944-8949, which is incorporated herein by reference. 11-Mercapto-1-undecanol (1.04 mmol) was titrated in anhydrous ethanol (30 mL) using a saturated iodine solution, the equivalence point was determined by the persisting light yellow color of diluted iodine. The resultant solution was concentrated to approximately 3 mL and 10 mL of Milli Q water was added the solution and then extracted to anhydrous diethyl ether (3×10 mL). To remove any traces of water the ether extract was further washed with saturated NaCl and dried over anhydrous Na₂SO₄. Ether was removed under reduced pressure. The resultant disulfide was removed as an oil. A solution of PFPA COOH (4-Azidotetrafluorobenzoic Acid) (synthesis of PFPA COOH performed according to the procedure of Keana, J. F. W.; Cai, S. X. The J. Org. Chem. 1990, 55, 3640-3647) (2.08 mmol) in anhydrous CH₂Cl₂ was cooled to 0° C. on ice. N,N′-dimethylaminopyridine (DMAP) (0.08 mmol) and 1-ethyl-3-(3-dimethylaminopropyl)carbodiimide hydrochloride (EDAC) (1.56 mmol) was added and the entire solution was cooled to 0° C. To the entire solution the disulfide was added and stirred at 0° C. for 1 hour and then made to return to room temperature. The solution was stirred for 12 hours. The product was extracted to anhydrous CH₂Cl₂ washed with saturated NaCl and dried over CH₂Cl₂. For further purification, flash column chromatography was performed where PFPA disulfide was eluted using 10/1 v/v hexane/ethyl acetate.

### Example 21

### Synthesis of PFPA Coupled Au Nanoparticles

Au nanoparticles were synthesized using Turkevitch's citrate reduction method as previously reported by Wang, X.; Ramstrom, O.; Yan, M. J. Mat. Chem. 2009, 19, 8944-8949. Au was obtained from HAuCl₄ salt (8.5 mg) and dissolved in 100 mL of Milli Q water. All glassware was previously washed in aqua regia (3HCl: 1HNO₃) and any impurities leading to nucleation removed. The solution was heated to boiling and 1.8 mL of 1% w/v Sodium Citrate was added at point of boiling. The solution was then cooled to room temperature while vigorously stirring. PFPA disulfide was added (5 mL, 1.7 mM) and stirred for approximately 10 hrs. The PFPA coupled Au nanoparticles were visualized using FTIR (Perkin-Elmer 2000 Fourier transform spectrometer) and the size was determined using DLS (approx 22 nm). The PFPA coupled Au nanoparticles were centrifuged at 12000 rpm for 10 minutes and all water was removed and redissolved in 100 mL Acetone. Au nanoparticles were washed couple of times in acetone to remove any trace PFPA disulfide. The PFPA coupled Au (PFPA-Au) was stored in acetone at room temperature and the volume was kept to 100 mL.

### Example 22

### Synthesis of Sugar Coupled PFPA-Au Nanoparticles

An array of sugar coupled PFPA-Au nanoparticles were created using the photocoupling technique described in Wang, X.; Ramstrom, O.; Yan, M. J. Mat. Chem. 2009, 19, 8944-8949. The sugar was photocoupled to Au nanoparticles via UV irradiation. A series of sugars, namely D-Lactose, D-Arabinose, D-Cellobiose, D-Saccharose (all sugars purchased from TCI America), was prepared as 2.9 mM aqueous solutions. A 1.1 mL volume of PFPA-Au nanoparticle acetone based solution was treated with 100 μL of 2.9 mM sugar in an aluminum foil covered short beaker where the opening was covered with a 280 nm cutoff filter and was exposed to UV light for 7 minutes while stirring. The sugar coupled Au-PFPA nanoparticles were then centrifuged at 10,000 rpm for 8 minutes and acetone was removed and the precipitated sugar coupled Au-PFPA was redissolved 1,500 μL of Milli Q water. The excess sugar was removed by overnight dialysis.

### Example 23

### Preparation of Training Matrices and Assays

Protein Bio Assay—

The unspecific binding sites of Sugar coupled Au-PFPA was inhibited by the addition of BSA (3% BSA, 0.2% Tween in PBS buffer pH 7). The 1,500 μL of sugar-coupled Au-PFPA was centrifuged at 9,000 rpm for 8 minutes and was redissolved in a PBS buffer with 3% BSA (0.2% Tween). The volume was made up to 900 μL. The resultant solution was incubated at room temperature with gentle shaking for 1 hour. The BSA blocked Au-PFPA-sugar nanoparticle was centrifuged at 10,000 rpm for 6 minutes and the excess BSA was removed. To a volume of 1,500 μL solution 10 mg/mL lectin (Con A—Concanavalin A, Canavalia ensiformis jack bean protein, SBA—soybean agglutinin, Glycine max and PNA—Peanut Agglutinin, Arachis hypogaea all purchased from Sigma Aldrich) was introduced the resultant solution was incubated at room temperature while gently shaking.

Training Matrix Using Weak Binding Sugars to Lectins—

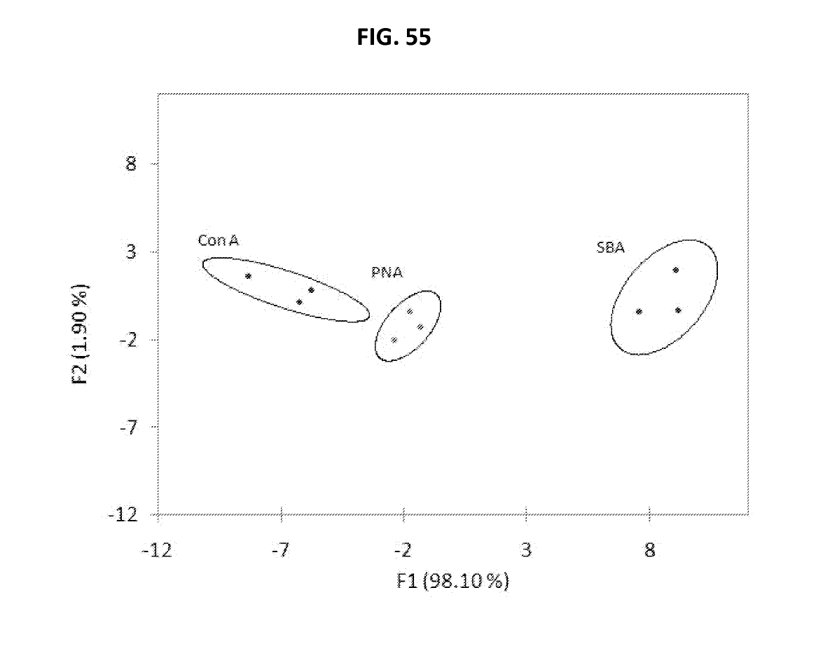

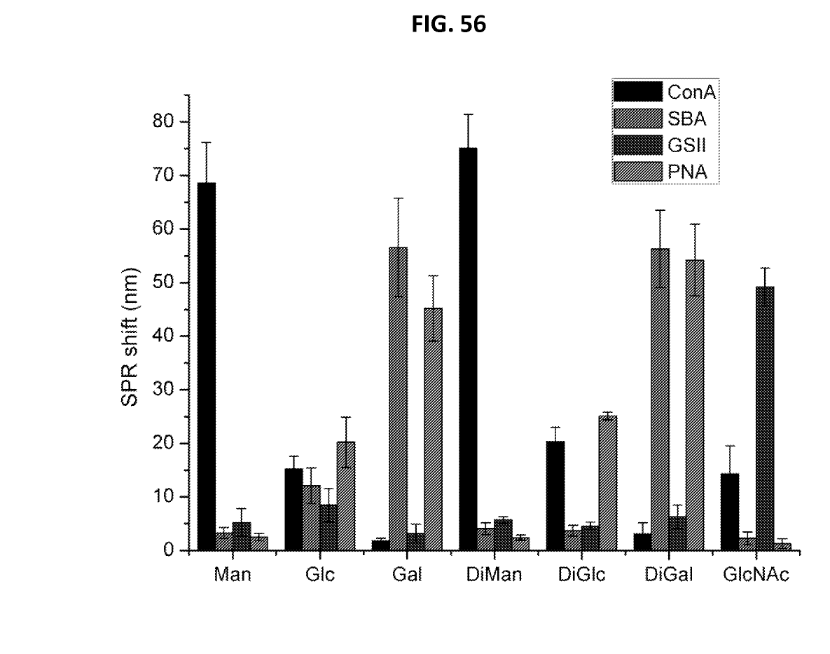

A training matrix was created using three lectins (Con A, SBA and PNA) and four types of sugar-bound Au-PFPA nanoparticles. Each assay was repeated three times, providing a 3×4×3 training matrix (Table 10). The factor scores (Zi from equation (1) or Fi) calculated for the training matrix from XLSTAT® were F1=98.10% and F2=1.90%. The F2 vs. F1 was plotted as shown in FIG. 55.

TABLE 10
Lectin - D-sugar (weak binding/unspecific sugars) training matrix

| Protein\Sugar | Lactose | Sucrose | Arabinose | Cellobiose |
|---|---|---|---|---|
| SBA | 1.5 | 0 | 5.1 | 0 |
| SBA | 3.7 | 1.1 | 5.9 | 0.8 |
| SBA | 1.1 | 1.4 | 4.5 | 2.1 |
| PNA | 2 | 4.9 | 1.8 | 2.2 |
| PNA | 2.4 | 1.4 | 1.6 | 1.8 |
| PNA | 3.7 | 2.4 | 2.1 | 1.3 |
| Con A | 6.2 | 4.8 | 1.9 | 1.1 |
| Con A | 5.1 | 2.4 | 0.8 | 1.8 |
| Con A | 7.9 | 2.8 | 1.1 | 0.1 |

Confusion Matrix:

Validation of this training set was carried out using a confusion matrix (resubstitution method). A confusion matrix is used in supervised learning in artificial intelligence to see if the system has the ability to classify cases without confusing them. The columns in the confusion matrix represent the actual classes while the rows represent the predicted classes. If the actual classes match the predicted classes, the matrix will show 100% classification. The confusion matrix that was created to determine the classification accuracy of the training matrix (Table 10) displayed 100% classification accuracy (Table 11). These results confirm that all nine training lectins (three lectins×three replicates) were classified with 100% classification accuracy. The Wilk's lamda (A) value for the training set was 0.0073. The 95% confidence ellipses show no overlapping and are within reasonable spatial separation of each other.

TABLE 11
Confusion matrix for the training set for training matrix in Table 10

| from \ to | Con A | PNA | SBA | Total | % correct |
|---|---|---|---|---|---|
| Con A | 3 | 0 | 0 | 3 | 100.00% |
| PNA | 0 | 3 | 0 | 3 | 100.00% |
| SBA | 0 | 0 | 3 | 3 | 100.00% |
| Total | 3 | 3 | 3 | 9 | 100.00% |

Training Matrix Using Strong Binding Sugars to Lectins—

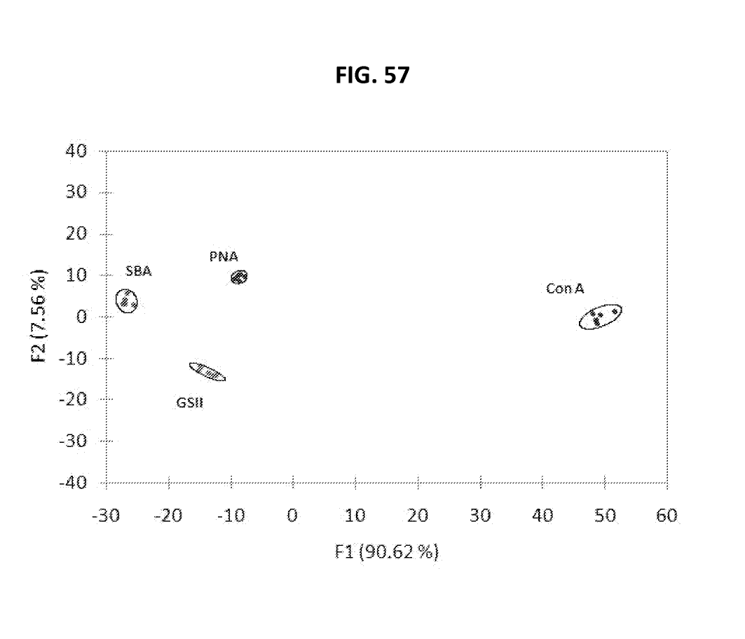

A study was also done to identify different lectins using specific binding interaction between lectins and sugars (Wang, X.; Ramstrom, O.; Yan, M. J. Mat. Chem. 2009, 19, 8944-8949). The lectins used in the study were Con A, SBA, PNA and GSII. The sugars used were D-Mannose, D-Glucose, D-Galactose, α-1,4-mannobiose, α-1,3-galactobiose and N-acetyl-D-glucosamine. These sugars are known to have specific binding to lectins (Jurs, P. Science 1986, 232, 1219-1224). The training matrix was created as four lectins×six sugars×five replicates (Table 12). The calculated factor scores were F1=90.62%, F2=7.56%, and F3=1.82%. Out of the three factors (F1, F2 and F3) the best separation of the 95% confidence ellipse was seen in the plot between F2 vs. F1, as shown in FIG. 57. The Wilk's lamda (A) value for the training set was 0.000. The classification proved to be 100% accurate and was validated using the confusion matrix of Table 13. The 95% confidence ellipses show clear spatial separation, and no overlap between the ellipses is visible. Unlike the weak binding interactions studied in before using non-specific binding sugars, the strong binding sugars show very good class separation.

TABLE 12
Lectin - sugar (specific) training matrix

| | Sugar | | | | | | |
|---|---|---|---|---|---|---|---|
| Protein | Man | Glc | Gal | DiMan | DiGlc | DiGal | GlcNAc |
| Con A | 74.9 | 13.7 | 1.7 | 69.3 | 19.3 | 1.5 | 9.4 |
| Con A | 73.4 | 13.4 | 2.1 | 77.4 | 20.1 | 2.2 | 8.1 |
| Con A | 69.8 | 14.7 | 2.4 | 81.4 | 16.4 | 5.3 | 17.8 |
| Con A | 69.1 | 14.9 | 1.2 | 67.6 | 23 | 1.1 | 19.8 |
| Con A | 55.8 | 19.3 | 1.6 | 79.8 | 22.7 | 5.4 | 16.4 |
| SBA | 2.3 | 15.4 | 68.2 | 5.4 | 3.3 | 50.6 | 1.4 |
| SBA | 2.5 | 8.8 | 47.2 | 4.8 | 2.9 | 62.3 | 3.6 |
| SBA | 3.9 | 8.4 | 48.2 | 3.2 | 3.8 | 50.9 | 2.2 |
| SBA | 3.1 | 14.2 | 56.1 | 2.9 | 3.1 | 51.9 | 3.4 |
| SBA | 4.7 | 13.7 | 63.3 | 4.2 | 5.4 | 65.8 | 0.9 |
| GSII | 3.1 | 11.2 | 4.2 | 5.2 | 4 | 7.4 | 48.2 |
| GSII | 4 | 12 | 1.8 | 6.5 | 4.1 | 4.7 | 49.2 |
| GSII | 7.4 | 8.2 | 1.1 | 5.9 | 5.5 | 8.2 | 53.4 |
| GSII | 2.9 | 4.7 | 4 | 4.9 | 5.2 | 7.9 | 44 |
| GSII | 8.6 | 6.4 | 4.9 | 6 | 4.2 | 3.2 | 51.2 |
| PNA | 1.7 | 18.2 | 49.2 | 2.1 | 24.3 | 42.5 | 0.8 |
| PNA | 3.2 | 24.7 | 51.3 | 2.5 | 26.1 | 59.2 | 2.6 |
| PNA | 2.8 | 24.9 | 39.2 | 2.7 | 25 | 57.3 | 1.6 |
| PNA | 1.8 | 19.4 | 38.2 | 1.7 | 24.6 | 57.4 | 1.4 |
| PNA | 3 | 13.8 | 48.1 | 3 | 25.5 | 54.6 | 0.1 |

TABLE 13
Confusion matrix for the training set of Table 12

| | to | | | | | |
|---|---|---|---|---|---|---|
| from | Con A | GSII | PNA | SBA | Total | % correct |
| Con A | 5 | 0 | 0 | 0 | 5 | 100.00% |
| GSII | 0 | 5 | 0 | 0 | 5 | 100.00% |
| PNA | 0 | 0 | 5 | 0 | 5 | 100.00% |
| SBA | 0 | 0 | 0 | 5 | 5 | 100.00% |
| Total | 5 | 5 | 5 | 5 | 20 | 100.00% |

### Statistical Analysis

Unknown Analysis:

LDA recognizes unknowns and correctly classifies them to their respective classes. Once the discriminant function has been derived, class predictions can be done by classification functions, (a classification function is not similar to discriminate function) where the classification function can be used to determine the predictive class of an unknown. There are as many classification functions as there are groups. Once classification scores are computed for each group, classification score is computed for each case in each group.

Unknown Analysis of the Weak Binding Lectins Using the Training Matrix—

To test the training matrix and its strength to identify individual lectins, seven blindly prepared lectins (U1-U7) were used as test samples. The procedure for each test sample is the same as that used for the known samples used in the training matrix. The final SPR shift (Table 14) was used in the training matrix (Table 10) for prediction. All seven samples were correctly classified (Table 15) using the classification function calculated for the training matrix (Table 10).

TABLE 14
Unknown SPR shifts of the weak binders/unspecific D-sugars

| Unknown Protein\Sugar | Lactose | Sucrose | Arabinose | Cellobiose |
|---|---|---|---|---|
| u1 | 1.6 | 0.5 | 4.5 | 2.1 |
| u2 | 1.1 | 0.5 | 5 | 1.8 |
| u3 | 5.5 | 3 | 1.1 | 2 |
| u4 | 5.7 | 2.5 | 1 | 1.8 |
| u5 | 2.5 | 2.1 | 1.9 | 2 |
| u6 | 1.6 | 0.5 | 4.5 | 2.1 |
| u7 | 1.1 | 0.5 | 4.5 | 2.1 |

TABLE 15
Predicted class analysis using seven unknowns

| Unknown protein | Predicted class | F1^{a} | F2^{a} |
|---|---|---|---|
| u1 | SBA | 7.6962 | 0.1923 |
| u2 | SBA | 9.3633 | −0.0267 |
| u3 | Con A | −6.0457 | 0.5649 |
| u4 | Con A | −6.2347 | 0.6765 |
| u5 | PNA | −0.9589 | −1.0710 |
| u6 | SBA | 7.6962 | 0.1923 |
| u7 | SBA | 8.0990 | −0.1930 |

^{a}F1 and F2 are factor scores

Unknown Analysis of the Strong Binding Lectins Using the Training Matrix—

Unknown lectin detection was carried out using four blindly prepared lectin samples (U1-U4). All five unknowns were classified with 100% accuracy using LDA. The unknown array is shown in Table 16 and the prediction is shown in Table 17.

TABLE 16
Array for 4 unknown samples using strong/specific binding sugars

| Unknown | Sugar | | | | | | |
|---|---|---|---|---|---|---|---|
| Protein | Man | Glc | Gal | DiMan | DiGlc | DiGal | GlcNAc |
| u1 | 52.2 | 20.1 | 5.8 | 61.3 | 29.9 | 3.3 | 16.8 |
| u2 | 6.7 | 15.6 | 43.6 | 9.6 | 8.1 | 43.5 | 5.4 |
| u3 | 16.3 | 19.2 | 15.2 | 10.1 | 9 | 13.2 | 38 |
| u4 | 7.7 | 27.6 | 41 | 9.9 | 27.9 | 43.3 | 6.6 |

TABLE 17
Factor scores for each unknown

| Unknown protein | Predicted class | F1 | F2 | F3 |
|---|---|---|---|---|
| u1 | Con A | 45.8484 | 2.2598 | 4.9875 |
| u2 | SBA | −16.6159 | 2.8648 | −2.8304 |
| u3 | GSII | −6.1031 | −8.0070 | 1.9061 |
| u4 | PNA | 0.7500 | 8.3899 | 6.8254 |

### Example 24

### Studies of Prostate Cancer Cells

Synthesis of lactose-conjugated fluorescent silica nanoparticles: The FSNPs were prepared using a dye precursor prepared from fluorescein isothiocyanate isomer I (FITC) and 3-aminopropyltriethoxysilane. The dye precursor (5 mL) was mixed with TEOS (2.8 mL) and added to 200 proof EtOH (34 mL) followed by the addition of NH₄OH (2.8 mL). The reaction was allowed to proceed at room temperature for at least 8 h with vigorous stirring. PFPA-silane was synthesized following a previously reported procedure and was added directly to the Stöber solution, then the mixture was stirred at room temperature overnight. The next day the mixture was brought to reflux while continuing stirring for 1 h at −78° C. to facilitate covalent bonding of the silane to the silica. Functionalized silica nanoparticles suspension was centrifuged (10 min, 8000 rpm) and was re-dispersed in the fresh solvent by sonication. This centrifugation/re-dispersion procedure was repeated three times with EtOH and twice with acetone. A solution of PFPA-functionalized SiO₂ NPs in acetone was placed in a flat-bottomed dish, and an aqueous solution of carbohydrate was added. The mixture was covered with a 280-nm long-path optical filter and was irradiated with a 450-W medium pressure Hg lamp for 10 min under vigorous stirring. Centrifugation of the solution at 8,000 rpm for 10 min separated the carbohydrate-attached SiO₂ NPs as precipitates. Excess carbohydrate was removed by membrane dialysis in water for 24 hours. The resulting carbohydrate-conjugated fluorescent silica nanoparticles were soaked in a BSA solution (0.3 wt %) at 4° C. for 1 h. The excess BSA in the solution was then removed by centrifugation and sonication in Milli-Q water, 3 times.

Fluorescent Imaging and Fluorescent Intensity Measurement of Prostate Cancer Cells Bound with Lactose-Conjugated Silica Nanoparticles:

Typically, 1×10⁶ cells were cultured in a tissue culture dish and stored in an incubator for 2 days. After removal of the growth medium, cells were washed three times with PBS and carbohydrate-conjugated FSNPs were added into the dishes. For these embodiments investigating the role of DTT in affecting bindings between NPs and cells, the cells were pretreated with DTT-contained PBS before soaking in NP solutions. After incubation, a portion of the supernatant was withdrew and measured by a spectrofluorometer to obtain the fluorescent intensity, which was compared with that of the original solutions before incubation. Meanwhile, cells were washed with HEPES buffer (pH 7.5) and imaged by a fluorescent microscope. To measure the fluorescent intensity of cells bound with carbohydrate-conjugated FSNPs, cells were detached from tissue culture dish by trypsinization. After centrifugation and removal of trypsin, cells were washed and re-suspended in HEPES solutions. The fluorescent intensities of cells were then measured by a spectrofluorometer.

Western-Blotting Analysis of Galectin-1 after being Mixed with Lactose-Conjugated Silica Nanoparticle:

500 ng of Gal-1 (2 μL) was mixed with lactose-conjugated FSNPs (30 μL) at 4° C. for 1 h. The concentrations of NP solution varied from 0.17 to 3400 μg/mL. After centrifugation at 13,000 rpm (4° C.) for 10 min, 25 μL of supernatant was subjected to electrophoresis on a 15% SDS-polyacrylamide gel and transferred to PVDF membrane. Membranes were blocked at room temperature for 1 h with block buffer and incubated with mouse monoclonal antibodies to galectin-1 overnight at 4° C. After being washed with Milli-Q water for three times, the membrane was incubated with goat anti-mouse IgG antibodies at room temperature for 1 h. After drying, the dye-labeled membrane was then imaged by an infrared imaging system. The weight of Gal-1 bound to FSNPs was calculated by measuring the optical densities of each blot and the subsequent normalizations with control samples, which contained 500 ng of Gal-1 without being mixed with FSNPs.

### Example 25

### Plural Microarray Embodiments

Fabrication of Lectin Super-Microarrays:

The lectin super-microarrays were prepared on epoxy-coated glass slides. Glass slides were firstly cleaned in Piranha solution (conc. H₂SO₄/H₂O₂, 1:1, v:v) by heating the slides at 80° C. for 1 h followed by thorough rinsing with boiling water 3 times. The clean slides were soaked in a toluene solution of 3-glycidyloxypropyltrimethoxysilane (12.6 mM, >95%, TCI America; Portland, Oreg.) for 4 hours, rinsed thoroughly with toluene and dried with nitrogen. Con A (Concanavalin A), BSA (Bovine serum albumin), SBA (Soybean Agglutinin), PNA (Peanut Agglutinin), BS-I (Bandeiraea simplicifolia) (Sigma-Aldrich; St. Louis, Mo.), DBA (Dolichos Biflorus Agglutinin), UEA (Ulex Europaeus Agglutinin I), WGA (Wheat Germ Agglutinin) and RCA (Ricinus Communis Agglutinin I) (Vector Laboratories; Burlingame, Calif.) were used as received. The concentration of Con A was calibrated spectrophotometrically using A1%, 1 cm (280 nm)=13.7 at pH 7.2.47 CV-N (Cyanovirin-N) and OAA (Oscillatoria Agardhii Agglutinin) wild-type lectins and CN-V mutants CVN^{Q50C} and CVN^{MutDB} were supplied by Professor Angela M. Gronenborn's research laboratory at University of Pittsburgh. The lectin solutions were prepared in phosphate-buffered saline at concentrations of 1 mg/mL except for PNA and RCA which were prepared at 0.25 mg/mL since the two solutions were more viscous than other lectin solutions. Glycerol (40%) was added to the buffer solution to prevent complete drying of the liquid droplets after printing. A volume of 0.3 mL of each lectin solution was transferred to the microtiter plate well and the solutions were spotted onto the epoxy-coated glass slides using a robotic printer (BioOdyssey Calligrapher Mini-arrayer; Bio-Rad Laboratories, Inc.) and a 360-μm capillary pin. Each lectin was printed with 5 duplicate spots and each spot was printed 5 times. The glass slides were then incubated in a humid chamber (80% humidity) at 25° C. for 3 h to facilitate the immobilization of the lectins. A SecureSeal™ hybridization chambers sheet, i.e., PDMS isolator (Grace Bio-lab, Bend, Oreg.), was carefully placed on the glass slide to create 16 individual wells. A blocking solution of BSA in pH 7.4 PBS buffer (1%) was then added to each well and the slide was incubated at room temperature for 1 h, rinsed with the PBS buffer and was dried with N₂.

Treating Super-Microarray with Glyco-FSNP:

The lectin super-microarrays were incubated in the solution of glycol-FSNPs in HEPES (1.5 mg/mL) for 2 h, and were then gently rinsed with the HEPES buffer containing 0.1% Tween 20 for 3 times and dried with N₂. For the competition assays, free ligand 2α-Man2 (1 nM-1 mM) was added to FSNP-Man2 (1.5 mg/mL) before the solution was added to each well for incubation. The resulting slides were imaged under a microarray scanner (GenePix 4100A, Molecular Devices, Inc) at 532 nm excitation. The fluorescence images were recorded and the data were analyzed using the supplied software (Axon GenePix Pro 5.1)

In view of the many possible embodiments to which the principles of the disclosed invention may be applied, it should be recognized that the illustrated embodiments only exemplary features of the invention and should not be taken as limiting the scope of the invention. Rather, the scope of the invention is defined by the following claims. We therefore claim as our invention all that comes within the scope and spirit of these claims.

1. A method for labeling a molecule with a signal generating moiety, comprising:
providing a nanoparticle comprising a signal generating moiety;
coupling the nanoparticle to a perhalophenyl azide to form a nanoparticle-azide conjugate; and
labeling the molecule with the nanoparticle-azide conjugate.

2. The method according to claim 1 where providing a nanoparticle comprising a signal generating moiety comprises making a nanoparticle comprising a signal generating moiety.

3. The method according to claim 2 where making comprises co-condensing a fluorescent dye with a monomer useful for making a nanoparticle.

4. The method according to claim 1 where the signal generating moiety is a fluorescent dye.

5. The method according to claim 1 where the perhalophenylazide has a formula 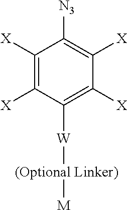
where X is a halogen; W is aliphatic, aryl, and a heteroatom-containing moiety; the optional linker may be selected from aliphatic, aryl, and a heteroatom-containing moiety; and M may be selected a metal-, metalloid-, and non-metal-containing moiety.

6. The method according to claim 5 where X is selected from chlorine, bromine, fluorine, chlorine, and combinations thereof.

7. The method according to claim 5 where W is a hetereoatom-containing moiety selected from heteroaryl, carbonate (—OC(O)OR^{a}), ether (—OR^{a}), ester (—C(O)OR^{a}), ketone (—C(O)R^{a}), peroxy (—OOR^{a}), phosphine (—PR^{a}R^{b}R^{c}), sulfinyl (—S(O)R^{a}), sulfonyl (—SO₂R^{a}), carbonothioyl (—C(S)R^{a}), oxazole, oxadiazole, imidazole, triazole, tetrazole, amide (—C(O)NR^{a}R^{b}), azo (—NNR^{a}), imide (—C(O)NR^{a}C(O)R^{b}), isonitrile (—NC), amine (—NHR^{a}, —NR^{a}R^{b}), —N-maleimido, —NH-biotinyl, and —CONH-A-S—S—B—NH-biotinyl (where A and B are spacer atoms and the S—S bond is reductively cleaved at a later stage) where R^{a}, R^{b}, and R^{c} individually are selected from hydrogen, aliphatic, aryl, a heteroatom-containing moiety, and any combination thereof.

8. The method according to claim 5 where the optional linker is selected from aliphatic groups, aryl groups, and a heteroatom-containing moiety.

9. The method according to claim 5 where the linker is (CH₂)_{q}, where q ranges from 0 to about 20, or an alkylene glycol.

10. The method according to claim 5 where M is selected from a titanium-containing moiety, a zirconium-containing moiety, a zinc-containing moiety, a silicon-containing moiety, a boron-containing moiety, a phosphorus-containing moiety, a sulfur-containing moiety, and a selenium-containing moiety.

11. The method according to claim 10 where M is selected from silyl, silyl ether, titanyl, titanyl ether, phosphate (—OP(O)(OH)₂), phosphoryl (—P(O)(OH)₂), phosphine (—PR^{a}R^{b}R^{c}), where R^{a}, R^{b}, and R^{c} individually are selected from hydrogen, aliphatic, aryl, a heteroatom-containing moiety, and any combination thereof; and thiols.

12. The method according to claim 5 where the perhalophenylazide has a formula

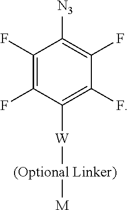

13. The method according to claim 5 where the perhalophenylazide has any one of the following formulas 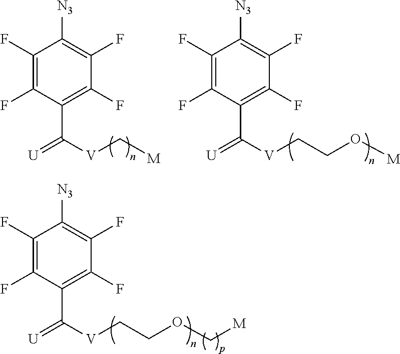
where U is selected from O, S, NH, and NR^{a}, where R^{a} is selected from hydrogen, aliphatic, aryl, a heteroatom-containing moiety, and any combination thereof; V is selected from O, S, NH, and NR^{a}; and n and p individually range from 0 to about 20.

14. The method according to claim 13 where n and p individual range from about 1 to about 10.

15. The method according to claim 5 where the perhalophenylazide has the following formulas 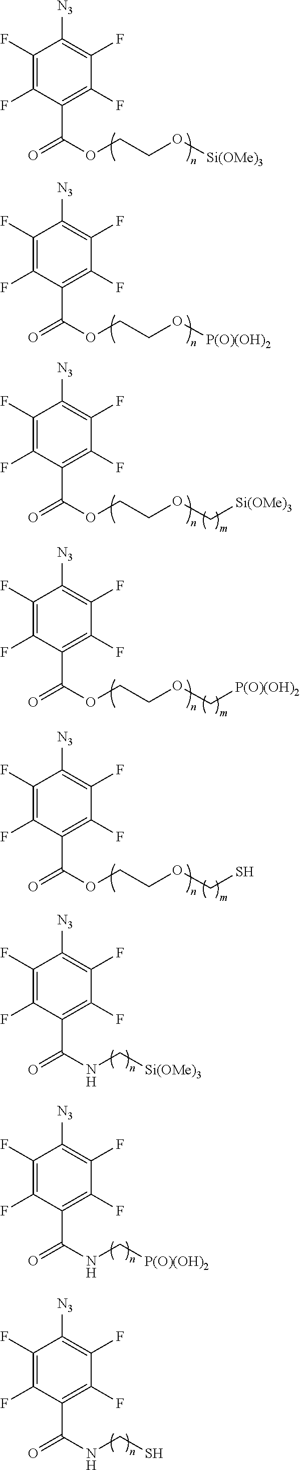
where n and p individual range from about 1 to about 10.

16. The method according to claim 5 where the perahalophenylazide has the following structures

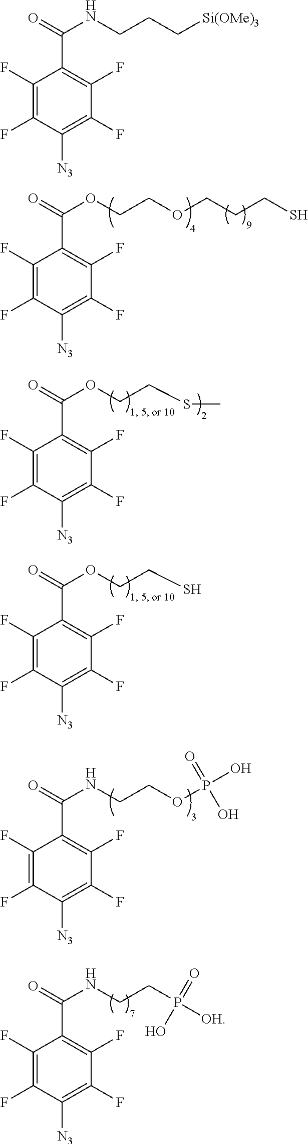

17. The method according to claim 1 where the perhalophenylazide has a formula 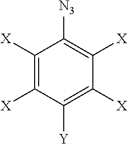
wherein X may be a halogen, and Y may be selected from a heteroatom-containing moiety capable of undergoing further chemical manipulation to make a perhalophenylazide moiety.

18. The method according to claim 17 where X is fluorine, chlorine, and combinations thereof.

19. The method according to claim 17 where Y is selected from heteroaryl, halogen, (iodine, bromine, chlorine, and fluorine), aldehyde (—CHO), acyl halide ([—C(O)X], where X may be selected from fluorine, chlorine, bromine, and iodine), carbonate (—OC(O)OR^{a}), carboxyl (—C(O)OH), carboxylate (—COO⁻), ether (—OR^{a}), ester (—C(O)OR^{a}), hydroxyl (—OH), ketone (—C(O)R^{a}), peroxy (—OOR^{a}), hydroperoxy (—OOH), phosphate (—OP(O)(OH)₂), phosphoryl (—P(O)(OH)₂), phosphine (—PR^{a}R^{b}R^{c}), sulfinyl (—S(O)R^{a}), sulfonyl (—SO₂R^{a}), carbonothioyl (—C(S)R^{a} or —C(S)H), sulfino (—S(O)OH), sulfo (—SO₂OH), thiocyanate (—SCN), isothiocyanate (—NCS), oxazole, oxadiazole, imidazole, triazole, tetrazole, amide (—C(O)NR^{a}R^{b}), azide (—N₃), azo (—NNR^{a}), cyano (—OCN), isocyanate (—NCO), imide (—C(O)NR^{a}C(O)R^{b}), nitrile (—CN), isonitrile (—NC), nitro (—NO₂), nitroso (—NO), nitromethyl (—CH₂NO₂), amine (—NH₂, —NHR^{a}, —NR^{a}R^{b}), —N-maleimido, —NH-biotinyl, —CONH-A-S—S—B—NH-biotinyl (where A and B are spacer atoms and the S—S bond is reductively cleaved at a later stage), and any homologated derivatives thereof, where R^{a}, R^{b}, and R^{c} individually are selected from hydrogen, aliphatic, aryl, a heteroatom-containing moiety, and any combination thereof.

20. The method according to claim 1 where the perhalophenylazide can be further modified using a moiety having a general formula illustrated below
Z-(Optional Linker)-M
where Z is a nucleophilic group or an electrophilic group; the optional linker is selected from aliphatic, aryl, or a heteroatom-containing moiety; and M is a metal-containing moiety, a metalloid-containing moiety, and a non-metal containing moiety.

21. The method according to claim 20 where Z is nucleophilic and is selected from hydroxyl (R^{a}OH), thiol (R^{a}SH), amine (NH₂, NHR^{a}, NR^{a}R^{b}), the anions formed from these groups, alkyl lithium moieties (LiCR^{a}R^{b}R^{c}), metal-containing compounds (e.g. MgCR^{a}R^{b}R^{c} and SnCR^{a}R^{b}R^{c}), and boronic acids, where R^{a}, R^{b}, and R^{c} individually are selected from hydrogen, aliphatic, aryl, a heteroatom-containing moiety, and any combination thereof.

22. The method according to claim 20 where Z is electrophilic and is selected from aldehyde (R^{a}CHO), acyl halide ([RC(O)X], where X is selected from fluorine, chlorine, bromine, and iodine), carbonate (R^{a}OC(O)OR^{b}), carboxyl (R^{a}C(O)OH), ester (R^{a}C(O)OR^{b}), ketone (R^{a}C(O)R^{b}), sulfinyl (R^{a}S(O)R^{b}), sulfonyl (R^{a}SO₂R^{b}), carbonothioyl (R^{a}C(S)R^{b} or R^{a}C(S)H), sulfino (R^{a}S(O)OH), sulfo (R^{a}SO₃H), amide (R^{a}C(O)NR^{b}R^{c}), cyano (R^{a}OCN), isocyanate (R^{a}NCO), imide (R^{a}C(O)NR^{b}C(O)R^{c}), and nitrile (R^{a}CN); R^{a}, R^{b}, R^{c}, and R^{d} independently are hydrogen, aliphatic, aryl, heteroaliphatic, heteroaryl, a polypeptide, and any combination thereof.

23. The method according to claim 20 where M is selected from a titanium-containing moiety, a zirconium-containing moiety, a zinc-containing moiety, a silicon-containing moiety, a boron-containing moiety, a phosphorus-containing moiety, a sulfur-containing moiety, and a selenium-containing moiety.

24. The method according to claim 23 where M is selected from silyl, silyl ether, titanyl, titanyl ether, phosphate (—OP(O)(OH)₂), phosphoryl (—P(O)(OH)₂), phosphine (—PR^{a}R^{b}R^{c}), where R^{a}, R^{b}, and R^{c} individually are selected from hydrogen, liphatic, aryl, a heteroatom-containing moiety, and any combination thereof; and thiols

25. The method according to claim 20 where the optional linker is —(CH₂)_{q}—, where q ranges from 0 to about 20, or an alkylene glycol.

26. The method according to claim 1 where the nanoparticle is a silica nanoparticle or a titania nanoparticle.

27. The method according to claim 1 where coupling comprises exposing the nanoparticle and perhalophenyl azide to a reaction energy source.

28. The method according to claim 27 where the reaction energy source is ultraviolet light.

29. The method according to claim 1 where the molecule is a biological molecule.

30. The method according to claim 1 where the molecule is a carbohydrate, an amino acid, an amino acid oligomer, a protein, a nucleic acid, a nucleic acid oligomer, RNA or DNA.

31. The method according to claim 2 where the molecule is a glycan.

32. The method according to claim 1 where the molecule is a therapeutic agent.

33. The method according to claim 1 comprising high throughput synthesis.

34. The method according to claim 33 comprising using microarrays to synthesize libraries of dye-entrapped, nanoparticle-labeled materials.

35-54. (canceled)

55. A method for making a microarray, comprising:
providing a solid support; and
immobilizing at least two different glycan binding proteins or two different carbohydrates on the solid support using a PHPA.

56. The method according to claim 55 further comprising modifying the substrate surface to prevent or substantially preclude interactions between biological molecules and the substrate.

57. The method according to claim 55 further comprising increasing signals resulting from specific interactions and decreasing background noises due to non-specific adsorption.

58. The method according to claim 55 comprising using polymer-based PHPA surfaces to enhance specific interaction signals.

59. The method according to claim 58 comprising using polymer-PHPA surfaces, providing high-density ligands and an antifouling coating.

60. The method according to claim 59 where ligands and an anti-fouling coating are applied at designated locations in a spatially-controlled fashion.

61. The method according to claim 55, comprising:
applying carbohydrates on a PAAm-PFPA surface;
coating polystyrene from a solution onto the surface; and
irradiating the surface to attach both carbohydrate ligands and a polyalkylene polymer to the surface to produce a carbohydrate array having a background covered with polyalkylene.

62. The method according to claim 59 where the polymer surface is a polyamino acid.

63. The method according to the claim 62 comprising using poly(L-lysine) (PLL) to enhance interactions between immobilized ligands and proteins.

64. The method according to claim 59 where the polymer is poly(allylamine) (PAAm)

65. The method according to claim 64 where PAAm-based PFPA are prepared by covalently immobilizing PAAm on PFPA-functionalized substrates using a PFPA-silane.

66-131. (canceled)

132. A method for differentiating and classifying cell lines, comprising:
exposing the cell lines to a microarray of perhalophenylazide-derived nanoparticle probes having a formula 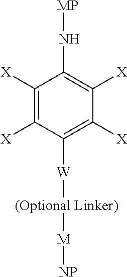
where X is a halogen; MP is a molecular probe selected from an antibody, a carbohydrate, an amino acid, an amino acid oligomer, a protein, a nucleic acid, a nucleic acid oligomer, RNA, DNA, a lipid, and combinations thereof; W is selected from aliphatic, aryl, and a heteroatom-containing moiety; optional linker is selected from aliphatic, aryl, and a heteroatom-containing moiety; M is selected from a metal-, metalloid-, and non-metal-containing moiety; and NP is a nanoparticle selected from a silica nanoparticle, a titania nanoparticle, a zinc oxide nanoparticle, a yttrium vanadium oxide nanoparticle, a gold nanoparticle, a silver nanoparticle, a lanthanum phosphate nanoparticle, a polystyrene nanoparticle, a graphene nanoparticle, and combinations thereof;
detecting a signal produced by a specific or non-specific interaction between the perhalophenylazide-derived nanoparticle probes and the cell lines; and
converting the signal to a score plot using linear discriminant analysis.
